(19) United States
(12) Patent Application Publication
(10) Pub. No.: US 2026/0055422 A1
(43) Pub. Date: Feb. 26, 2026

(54) METHODS AND COMPOSITIONS FOR THE INTRODUCTION AND REGULATED EXPRESSION OF GENES IN PLANTS

(71) Applicant: PIONEER HI-BRED INTERNATIONAL, INC., JOHNSTON, IA (US)

(72) Inventors: WILLIAM JAMES GORDON-KAMM, URBANDALE, IA (US); THEODORE M. KLEIN, WILMINGTON, DE (US); KEITH S. LOWE, JOHNSTON, IA (US); KEVIN E. MCBRIDE, DAVIS, CA (US); CHRISTOPHER J. SCELONGE, ANKENY, IA (US); NING WANG, JOHNSTON, IA (US)

(73) Assignee: PIONEER HI-BRED INTERNATIONAL, INC., JOHNSTON, IA (US)

(21) Appl. No.: 19/372,398

(22) Filed: Oct. 29, 2025

Related U.S. Application Data
(63) Continuation of application No. 18/047,714, filed on Oct. 19, 2022, now Pat. No. 12,497,628.
Application No. 18/047,714 is a continuation of application No. 17/181,136, filed on Feb. 22, 2021, now Pat. No. 11,512,321.

(51) Int. Cl. C12N 15/82

(52) U.S. Cl. CPC C12N 15/8241; C12N 15/8213; C12N 15/8216; C12N 15/8262

(57) ABSTRACT

Compositions and methods are provided for the introduction and the regulated expression of genes in plants. Compositions include promoter constructs that provide a level of activity useful for the regulated expression of site-specific recombinases, while avoiding premature excision. Further provided are isolated polynucleotides encoding novel babyboom polypeptides, expression cassettes, and plants comprising the same. Methods for the introduction of genes into plants are provided, including methods for plastid transformation and methods for the transformation of tissues from mature seeds and leaves.

### CROSS-REFERENCE TO RELATED APPLICATIONS

This application is a continuation of and claims the benefit of U.S. application Ser. No. 18/047,714 filed on Oct. 19, 2022, which is continuation of U.S. application Ser. No. 17/181,136 filed on Feb. 22, 2021, now U.S. Pat. No. 11,512,321, which is continuation of U.S. application Ser. No. 15/673,609 filed Aug. 10, 2017, now U.S. Pat. No. 10,968,458, which is a continuation of and claims the benefit of U.S. application Ser. No. 14/087,775 filed Nov. 22, 2013, now U.S. Pat. No. 9,765,352, which is a continuation of and claims the benefit of U.S. application Ser. No. 12/982,180 filed Dec. 30, 2010, abandoned, which claims the benefit of U.S. Provisional Application No. 61/291,257, filed on Dec. 30, 2009, expired, the contents of each of which are hereby incorporated by reference in their entireties.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 3650-US-CNT4.xml created on Oct. 22, 2025, and having a size of 302,284 bytes and is filed concurrently with the specification. The sequence listing contained in this ASCII formatted document is part of the specification and is herein incorporated by reference in its entirety.

### FIELD OF THE INVENTION

The present invention is drawn to the field of plant genetics and molecular biology. More particularly, the compositions and methods are directed to the introduction and regulated expression of genes in plants.

### BACKGROUND OF THE INVENTION

Current transformation technology provides an opportunity to engineer plants with desired traits. Major advances in plant transformation have occurred over the last few years. However, most transformation methods rely on the introduction of polynucleotides into embryonic tissues that are rapidly proliferating. Methods that allow for the transformation of more mature tissues would save considerable time and money. Accordingly, methods are needed in the art to increase transformation efficiencies of plants and allow for the transformation of more mature tissues.

Further, it is often necessary to reduce the activity of a transgene because the transgene may negatively affect the growth or fertility of the plant. Recombination systems can be used to excise the transgene, wherein the expression of a site-specific recombinase is regulated by an inducible promoter. Often, these systems are associated with premature excision. Accordingly, methods are needed in the art to efficiently excise transgenes with limited premature excision.

### BRIEF SUMMARY OF THE INVENTION

Compositions and methods are provided for the introduction and regulated expression of genes in plants. Compositions include promoter constructs useful for regulated induction of expression of an operably linked coding sequence. In particular embodiments, the promoter construct comprises a maize rab17 promoter or an active variant or fragment thereof and an attachment B (attB) site. The modified rab17 promoter constructs find use in methods for regulating the expression of various coding sequences, including site-specific recombinases, which can minimize the premature excision of polynucleotides of interest in plants.

Further provided are methods for the transformation of plastids, such as chloroplasts, that involve the introduction of a heterologous polynucleotide encoding a cell proliferation factor, such as a babyboom (BBM) polypeptide. Novel BBM sequences are provided, along with methods of introducing the sequences into plants and plants comprising the novel BBM sequences. Methods for preparing and transforming mature embryo explants and leaf tissues are also provided.

### BRIEF DESCRIPTION OF THE FIGURES

FIG. 1 provides a depiction of a phylogenetic analysis of 50 sequences with homology to maize babyboom (BBM).

FIGS. 2A-2M show the consensus motif sequences 1-10, 14, 15, and 19, respectively, discovered in the analysis described herein, along with the alignments of the regions of various polypeptides used to generate the consensus motifs.

FIG. 3 depicts the motifs found within 50 sequences with homology to maize BBM (ZmBBM).

FIGS. 4-1 through 4-3 show an alignment of the amino acid sequence of various BBM polypeptides: maize babyboom 2 (ZmBBM2; SEQ ID NO: 12), sorghum babyboom 2 (SbBBM2; SEQ ID NO: 28), rice babyboom 2 (OsBBM2; SEQ ID NO: 18), rice babyboom 3 (OsBBM3; SEQ ID NO: 20), rice babyboom 1 (OsBBM1; SEQ ID NO: 16), maize babyboom (ZmBBM; SEQ ID NO: 10), sorghum babyboom (SbBBM; SEQ ID NO: 4), rice babyboom (OsBBM; SEQ ID NO: 14), Brassica babyboom 1 (BnBBM1; SEQ ID NO: 24), Brassica babyboom 2 (BnBBM2; SEQ ID NO: 26), Arabidopsis babyboom (AtBBM; SEQ ID NO: 22), medicago babyboom (MtBBM; SEQ ID NO: 8), soybean babyboom (GmBBM; SEQ ID NO: 2), and grape babyboom (VvBBM; SEQ ID NO: 6).

FIG. 5 provides a depiction of the motifs found in babyboom polypeptides.

### DETAILED DESCRIPTION OF THE INVENTION

The presently disclosed compositions and methods are useful for the introduction and the regulated expression of genes in plants. Compositions comprise promoter constructs that provide a level of activity useful for the regulated expression of various coding sequences, including site-specific recombinases. Further provided are compositions comprising novel babyboom (BBM) polynucleotide and polypeptide sequences and plants comprising the same. Methods for the introduction of genes into plants are provided, including methods for introducing novel BBM polynucleotides and polypeptides into plants, methods for the enhancement of plastid transformation, and methods for the transformation of tissues from mature seeds.

The expression cassette having the sequence set forth in SEQ ID NO: 45, which is comprised of the maize rab17 promoter, an attB site, and the coding sequence for the site-specific recombinase FLP, is capable of expressing FLP upon induction in such a manner as to reduce premature excision. Without being bound by any theory or mechanism of action, it is believed that the presence of the attB site modifies the activity of the promoter, allowing for a tightly regulated induction of expression of an operably linked coding sequence. Therefore, compositions include promoter constructs comprising a modified maize rab17 promoter or an active variant or fragment thereof. In some of these embodiments, the promoter construct comprises the maize rab17 promoter or an active variant or fragment thereof and an attB site or a variant or fragment thereof. In some of these embodiments, the maize rab17 promoter has the sequence set forth in SEQ ID NO: 29 or an active variant or fragment thereof.

As used herein, the term “promoter” includes reference to a region of DNA involved in the recognition and binding of RNA polymerase and other proteins to initiate transcription of a coding sequence. Promoters may be naturally occurring promoters, a variant or fragment thereof, or synthetically derived. A “promoter construct” is a polynucleotide comprising a promoter and optionally, sequences that are not necessary for transcription initiation or part of the coding sequence and are located in between the promoter and the coding sequence in an expression cassette. These intervening sequences can include modulators, restriction sites, sequences of the 5′-untranslated region (5′-UTR), which is the region of a transcript that is transcribed, but is not translated into a polypeptide, and recombination sites.

The promoter in the promoter constructs is the maize rab17 promoter or an active variant or fragment thereof. The maize rab17 (responsive to abscisic acid) gene (GenBank Accession No. X15994; Vilardell et al. (1990) Plant Mol Biol 14:423-432; Vilardell et al. (1991) Plant Mol Biol 17:985-993; each of which is herein incorporated in its entirety) is expressed in late embryos, but its expression can be induced by exposure to abscisic acid or water stress. The sequence of the maize rab17 promoter corresponds to nucleotides 1-558 of GenBank Accession No. X15994, which was disclosed in Vilardell et al. (1990) Plant Mol Biol 14:423-432 and is set forth in SEQ ID NO: 126. An alternative maize rab17 promoter was disclosed in U.S. Pat. Nos. 7,253,000 and 7,491,813, each of which is herein incorporated by reference in its entirety, and is set forth in SEQ ID NO: 29. The rab17 promoter contains 5 putative abscisic acid responsive elements (ABRE) (Busk et al. (1997) Plant J 11:1285-1295, which is herein incorporated by reference in its entirety). The putative ABRE elements can be found at about −208 to −203 (nucleotides 304 to 309 of SEQ ID NO: 29), −162 to −157 (nucleotides 348 to 353 of SEQ ID NO: 29), −147 to −142 (nucleotides 363 to 368 of SEQ ID NO: 29), −141 to −136 (nucleotides 369 to 374 of SEQ ID NO: 29), and −96 to −91 (nucleotides 414 to 419 of SEQ ID NO: 29) in the maize rab17 promoter. The rab17 promoter also contains drought-responsive elements (DRE), of which the core sequence is identical to the DRE (drought-responsive) and CRT (cold-response elements) elements in Arabidopsis. The drought-responsive elements are found at −213 to −206 (nucleotides 299 to 306 of SEQ ID NO: 29) and −190 to −185 (nucleotides 322 to 327 of SEQ ID NO: 29) of the maize rab17 promoter. The CAAT and TATAA box can be found from nucleotides 395 to 398 and 479 to 484 of SEQ ID NO: 29, respectively.

In some embodiments, the maize rab17 promoter that is part of the presently disclosed promoter constructs has the sequence set forth in SEQ ID NO: 29 or an active variant or fragment thereof. In other embodiments, the maize rab17 promoter that is part of the presently disclosed promoter constructs has the sequence set forth in SEQ ID NO: 125 or 126 or an active variant or fragment thereof.

In some embodiments of the methods and compositions, the promoter constructs comprise active variants or fragments of the maize rab17 promoter. An active variant or fragment of a maize rab17 promoter (e.g., SEQ ID NO: 29, 125, 126) is a polynucleotide variant or fragment that retains the ability to initiate transcription. In some embodiments, an active fragment of a maize rab17 promoter may comprise at least about 50, 100, 150, 200, 250, 300, 350, 400, 450, or 500 contiguous nucleotides of SEQ ID NO: 29, 125, or 126, or may have at least about 40%, 45%, 50%, 55%, 60%, 65%, 70%, 75%, 80%, 85%0, 90%, 91%, 92%, 93%, 94%, 95%, 96%, 97%, 98%, 99% or more sequence identity to SEQ ID NO: 29, 125, or 126. In particular embodiments, an active variant or fragment of the maize rab17 promoter is one that is capable of initiating transcription in response to abscisic acid (ABA). In some of these embodiments, the promoter comprises at least one ABRE element. In particular embodiments, the promoter of the compositions and methods comprises from about −219 to about −102 of the maize rab17 promoter (corresponding to nucleotides 291 to 408 of SEQ ID NO: 29), which was shown to be sufficient to confer ABA responsiveness (Vilardell et al. (1991) Plant Mol Biol 17:985-993, which is herein incorporated by reference in its entirety).

In other embodiments, an active variant or fragment of the maize rab17 promoter is one that is capable of initiating transcription in response to desiccation. In some of these embodiments, the promoter comprises at least one DRE element.

In particular embodiments, the active maize rab17 promoter fragment comprises from about −219 to about −80 of the maize rab17 promoter (nucleotides 291 to 430 of SEQ ID NO: 29), which comprises all of the putative DRE and ABRE elements.

Without being bound by any theory or mechanism of action, it is believed that a promoter construct (the sequence of which is set forth in SEQ ID NO: 30) comprising a maize rab17 promoter and a site-specific attachment B (attB) site has a modified level of activity in comparison to the promoter in the absence of the attB site due to the presence and/or the location of the attB site relative to the promoter. Therefore, it is believed the attB site functions as a modulator of the maize rab17 promoter. Accordingly, promoter constructs comprising a maize rab17 promoter or a fragment or variant thereof, and an attB site are provided, and in some of these embodiments, the attB site modifies the activity of the promoter. In other embodiments, the promoter construct comprises a maize rab17 promoter or a fragment or variant thereof and a modulator that modifies the activity of the rab17 promoter.

As used herein, a “modulator” refers to a polynucleotide that when present between a promoter and a coding sequence, serves to increase or decrease the activity of the promoter. Non-limiting examples of modulators include recombination sites, operators, and insulators.

Attachment sites are site-specific recombination sites found in viral and bacterial genomes that facilitate the integration or excision of the viral genome into and out of its host genome. Non-limiting examples of a viral and bacterial host system that utilize attachment sites is the lambda bacteriophage and E. coli system (Weisberg and Landy (1983) In Lambda II, eds. Hendrix et al. (Cold Spring Harbor Laboratory, Cold Spring Harbor, N.Y.) pp. 211-250). The modulator of the promoter constructs can be an E. coli attachment site B (attB) site. The attB site can be naturally occurring E. coli attB sites or an active variants or fragments thereof or a synthetically derived sequence. Synthetically derived attB sites and active variants and fragments of naturally occurring attB sites are those that are capable of recombining with a bacteriophage lambda attachment P site, a process that is catalyzed by the bacteriophage lambda Integrase (Int) and the E. coli Integration Host Factor (IHF) proteins (Landy (1989) Ann Rev Biochem 58: 913-949, which is herein incorporated by reference in its entirety). AttB sites typically have a length of about 25 nucleotides, with a core 15-base pair sequence that is involved in the actual crossover event. Alternatively, active variants and fragments of naturally occurring attB sites are those that are capable of modulating the activity of a promoter when present within a promoter construct. Non-limiting examples of attB sites that can be used include attB1 (SEQ ID NO: 31), attB2 (SEQ ID NO: 32), attB3 (SEQ ID NO: 33), and attB4 (SEQ ID NO: 34), and variants or fragments thereof. In some embodiments, the modulator is an active variant or fragment of an attB site that is capable of modulating (i.e., increasing, decreasing) the activity of a promoter, but is not capable of recombination with an attachment P site. Non-limiting examples of such active variants of an attB site include those having the sequence set forth in SEQ ID NO: 107, 108, or 109.

In some embodiments, the distance of the modulator from the promoter impacts the ability of the modulator to modify the activity of the promoter. The modulator may be contiguous with the promoter and/or the polynucleotide of interest. In other embodiments, a linker sequence separates the promoter sequence and the modulator. As used herein, a “linker sequence” is a nucleotide sequence that functions to link one functional sequence with another without otherwise contributing to the expression or translation of a polynucleotide of interest when present in a promoter construct. Accordingly, the actual sequence of the linker sequence can vary. The linker sequence can comprise plasmid sequences, restriction sites, and/or regions of the 5′-untranslated region (5′-UTR) of the gene from which the promoter is derived. The linker sequence separating the promoter and the modulator can have a length of about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 400, 500, 1000 nucleotides or greater. In certain embodiments, a linker sequence of about 133 nucleotides separates the promoter and the modulator. In some embodiments, the linker sequence comprises a fragment of the rab17 5′-UTR. The fragment of the 5′-UTR can be about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 nucleotides, or greater, in length. In certain embodiments, the promoter construct comprises a linker sequence separating the promoter and the modulator that comprises 95 nucleotides of the maize rab17 5′-UTR. In some of these embodiments, the 95 nucleotide sequence has the sequence set forth in SEQ ID NO: 35. In certain embodiments, the linker sequence between the promoter and modulator has the sequence set forth in SEQ ID NO: 36 or a variant or fragment thereof.

In some embodiments, the promoter construct comprises a linker sequence separating the modulator and the polynucleotide of interest. The length and sequence of this linker may also vary and can be about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 400, 500, 1000 nucleotides or greater in length. In certain embodiments, a linker sequence of about 61 nucleotides separates the modulator and the polynucleotide of interest. In certain embodiments, the linker sequence between the modulator and the polynucleotide of interest has the sequence set forth in SEQ ID NO: 37 or a variant or fragment thereof. In other embodiments, a linker sequence of about 25 nucleotides separates the modulator and the polynucleotide of interest. In certain embodiments, the linker sequence between the modulator and the polynucleotide of interest has the sequence set forth in SEQ ID NO: 123.

In certain embodiments, the promoter construct has the sequence set forth in SEQ ID NO: 30 or a variant or fragment thereof.

The promoter constructs can be operably linked to a polynucleotide of interest that encodes a polynucleotide or polypeptide within an expression cassette. “Operably linked” denotes a functional linkage between two or more elements. For example, an operable linkage between a polynucleotide of interest and a promoter is a functional link that allows for expression of the polynucleotide of interest. Operably linked elements may be contiguous or non-contiguous. The expression cassette can comprise other 5′ or 3′ regulatory elements necessary for expression.

Regulatory elements that can be included in the expression cassette 5′ to the polynucleotide of interest include 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods or sequences known to enhance translation can also be utilized, for example, introns, and the like.

The expression cassette may also comprise a transcriptional and/or translational termination region functional in plants. The termination region may be native with the transcriptional initiation region (i.e., promoter), may be native with the operably linked polynucleotide of interest, may be native with the plant host, or may be derived from another source (i.e., foreign to the promoter, the polynucleotide of interest, the plant host, or any combination thereof). Convenient termination regions are available from the potato proteinase inhibitor (PinII) gene or the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639. In some embodiments, the pinII termination sequence has the sequence set forth in SEQ ID NO: 38 or an active variant or fragment thereof that is capable of terminating transcription and/or translation in a plant cell.

In certain embodiments, the expression cassette can comprise a recombination site, such as an attachment site 3′ to the polynucleotide of interest. In some of these embodiments, the recombination site is a second attB site. In some of those embodiments wherein the promoter comprises a first attB site, the second attB site following the polynucleotide of interest and the modulator attB are non-identical. In some of those embodiments wherein the modulator attB site is attB1 (SEQ ID NO: 31), the second attB site 3′ of the polynucleotide of interest can have the sequence set forth in SEQ ID NO: 31 (attB1), SEQ ID NO: 32 (attB2), SEQ ID NO: 33 (attB3), or SEQ ID NO: 34 (attB4), or an active variant or fragment thereof.

The recombination site 3′ to the polynucleotide of interest can be 5′ or 3′ to the termination region when present. The recombination site can be contiguous with the polynucleotide of interest and/or the termination sequence, if present. In some embodiments, however, a linker sequence separates the polynucleotide of interest and the recombination site. The length of this linker sequence can vary, but in some embodiments, is about 1, 2, 3, 4, 5, 6, 7, 8, 9, 10, 11, 12, 13, 14, 15, 16, 17, 18, 19, 20, 25, 30, 35, 40, 45, 50, 60, 70, 80, or 90 nucleotides in length. In particular embodiments, the linker sequence separating the recombination site and the polynucleotide of interest is about 16 nucleotides. In certain embodiments, the recombination site and the polynucleotide of interest are separated by a linker sequence having the nucleotide sequence set forth in SEQ ID NO: 39, or a variant or fragment thereof. In other embodiments, the linker sequence separating the recombination site and the polynucleotide of interest is about 8 nucleotides. In certain embodiments, the recombination site and the polynucleotide of interest are separated by a linker sequence having the nucleotide sequence set forth in SEQ ID NO: 124, or a variant or fragment thereof.

In some of those embodiments wherein a termination region is present on the expression cassette and the expression cassette further comprises a recombination site 3′ to the polynucleotide of interest, the termination region is 3′ to the recombination site and a linker sequence separates the recombination site and the termination region. The length of this linker sequence can vary, but in some embodiments, is about 1, 2, 3, 4, 5, 6, 7, 8, 9, 10, 11, 12, 13, 14, 15, 16, 17, 18, 19, 20, 25, 30, 35, 40, 45, 50, 60, 70, 80, or 90 nucleotides in length. In particular embodiments, the linker sequence separating the recombination site and the termination region is about 14 nucleotides. In certain embodiments, the recombination site and the termination region are separated by a linker sequence having the nucleotide sequence set forth in SEQ ID NO: 40 or a variant or fragment thereof.

The expression cassettes comprise a presently disclosed promoter construct regulating the expression of a polynucleotide of interest. The polynucleotide of interest may be any polynucleotide that encodes a polynucleotide (e.g., antisense, siRNA) or encodes a polypeptide. Where appropriate, the polynucleotide(s) of interest may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

In some embodiments, the polynucleotide of interest comprises a polynucleotide that encodes a site-specific recombinase. A site-specific recombinase, also referred to herein as a recombinase, is a polypeptide that catalyzes conservative site-specific recombination between its compatible recombination sites, and includes native polypeptides as well as derivatives, variants and/or fragments that retain activity, and native polynucleotides, derivatives, variants, and/or fragments that encode a recombinase that retains activity. The recombinase used in the methods and compositions can be a native recombinase or a biologically active fragment or variant of the recombinase. For reviews of site-specific recombinases and their recognition sites, see Sauer (1994) Curr Op Biotechnol 5:521-527; and Sadowski (1993) FASEB 7:760-767, each of which is herein incorporated by reference in its entirety.

Any recombinase system can be used in the methods and compositions. Non-limiting examples of site-specific recombinases include FLP, Cre, SSV1, lambda Int, phi C31 Int, HK022, R, Gin, Tn1721, CinH, ParA, Tn5053, Bxb1, TP907-1, U153, and other site-specific recombinases known in the art, including those described in Thomson and Ow (2006) Genesis 44:465-476, which is herein incorporated by reference in its entirety. Examples of site-specific recombination systems used in plants can be found in U.S. Pat. Nos. 5,929,301, 6,175,056, 6,331,661; and International Application Publication Nos. WO 99/25821, WO 99/25855, WO 99/25841, and WO 99/25840, the contents of each are herein incorporated by reference.

In some embodiments, the polynucleotide of interest encodes a recombinase from the Integrase or Resolvase families, including biologically active variants and fragments thereof. The Integrase family of recombinases has over one hundred members and includes, for example, FLP, Cre, lambda integrase, and R. For other members of the Integrase family, see, for example, Esposito et al. (1997) Nucleic Acids Res 25:3605-3614; and Abremski et al. (1992) Protein Eng 5:87-91; each of which are herein incorporated by reference in its entirety. Other recombination systems include, for example, the Streptomycete bacteriophage phi C31 (Kuhstoss et al. (1991) J Mol Biol 20:897-908); the SSV1 site-specific recombination system from Sulfolobus shibatae (Maskhelishvili et al. (1993) Mol Gen Genet 237:334-342); and a retroviral integrase-based integration system (Tanaka et al. (1998) Gene 17:67-76). In some embodiments, the recombinase does not require cofactors or a supercoiled substrate. Such recombinases include Cre, FLP, or active variants or fragments thereof.

The FLP recombinase is a protein that catalyzes a site-specific reaction that is involved in amplifying the copy number of the two-micron plasmid of S. cerevisiae during DNA replication. FLP recombinase catalyzes site-specific recombination between two FRT sites. The FLP protein has been cloned and expressed (Cox (1993) Proc Natl Acad Sci USA 80:4223-4227, which is herein incorporated by reference in its entirety). The FLP recombinase for use in the methods and compositions may be derived from the genus Saccharomyces. In some embodiments, a recombinase polynucleotide modified to comprise more plant-preferred codons is used. A recombinant FLP enzyme encoded by a nucleotide sequence comprising maize preferred codons (FLPm) that catalyzes site-specific recombination events is known (the polynucleotide and polypeptide sequence of which is set forth in SEQ ID NO: 41 and 42, respectively; see, e.g., U.S. Pat. No. 5,929,301, which is herein incorporated by reference in its entirety). Additional functional variants and fragments of FLP are known (Buchholz et al. (1998) Nat Biotechnol 16:657-662; Hartung et al. (1998) J Biol Chem 273:22884-22891; Saxena et al. (1997) Biochim Biophys Acta 1340:187-204; Hartley et al. (1980) Nature 286:860-864; Voziyanov et al. (2002) Nucleic Acids Res 30:1656-1663; Zhu & Sadowski (1995) J Biol Chem 270:23044-23054; and U.S. Pat. No. 7,238,854, each of which is herein incorporated by reference in its entirety).

The bacteriophage recombinase Cre catalyzes site-specific recombination between two lox sites. The Cre recombinase is known (Guo et al. (1997) Nature 389:40-46; Abremski et al. (1984) J Biol Chem 259:1509-1514; Chen et al. (1996) Somat Cell Mol Genet 22:477-488; Shaikh et al. (1977) J Biol Chem 272:5695-5702; and, Buchholz et al. (1998) Nat Biotechnol 16:657-662, each of which is herein incorporated by reference in its entirety). Cre polynucleotide sequences may also be synthesized using plant-preferred codons, for example such sequences (moCre; the polynucleotide and polypeptide sequence of which is set forth in SEQ ID NO: 43 and 44, respectively) are described, for example, in International Application Publication No. WO 99/25840, which is herein incorporated by reference in its entirety. Variants of the Cre recombinase are known (see, for example U.S. Pat. No. 6,890,726; Rufer & Sauer (2002) Nucleic Acids Res 30:2764-2772; Wierzbicki et al. (1987) J Mol Biol 195:785-794; Petyuk et al. (2004) J Biol Chem 279:37040-37048; Hartung & Kisters-Woike (1998) J Biol Chem 273:22884-22891; Santoro & Schultz (2002) Proc Natl Acad Sci USA 99:4185-4190; Koresawa et al. (2000) J Biochem (Tokyo) 127:367-372; and Vergunst et al. (2000) Science 290:979-982, each of which are herein incorporated by reference in its entirety).

In some embodiments, the polynucleotide of interest encodes a chimeric recombinase. A chimeric recombinase is a recombinant fusion protein which is capable of catalyzing site-specific recombination between recombination sites that originate from different recombination systems. For example, if the set of recombination sites comprises a FRT site and a LoxP site, a chimeric FLP/Cre recombinase or active variant or fragment thereof can be used, or both recombinases may be separately provided. Methods for the production and use of such chimeric recombinases or active variants or fragments thereof are described, for example, in International Application Publication No. WO 99/25840; and Shaikh & Sadowski (2000) J Mol Biol 302:27-48, each of which are herein incorporated by reference in its entirety.

In other embodiments, a variant recombinase is used. Methods for modifying the kinetics, cofactor interaction and requirements, expression, optimal conditions, and/or recognition site specificity, and screening for activity of recombinases and variants are known, see for example Miller et al. (1980) Cell 20:721-9; Lange-Gustafson and Nash (1984) J Biol Chem 259:12724-32; Christ et al. (1998) J Mol Biol 288:825-36; Lorbach et al. (2000) J Mol Biol 296:1175-81; Vergunst et al. (2000) Science 290:979-82; Dorgai et al. (1995) J Mol Biol 252:178-88; Dorgai et al. (1998) J Mol Biol 277:1059-70; Yagu et al. (1995) J Mol Biol 252:163-7; Sclimente et al. (2001) Nucleic Acids Res 29:5044-51; Santoro and Schultze (2002) Proc Natl Acad Sci USA 99:4185-90; Buchholz and Stewart (2001) Nat Biotechnol 19:1047-52; Voziyanov et al. (2002) Nucleic Acids Res 30:1656-63; Voziyanov et al. (2003) J Mol Biol 326:65-76; Klippel et al. (1988) EMBO J 7:3983-9; Arnold et al. (1999) EMBO J 18:1407-14; and International Application Publication Nos. WO 03/08045, WO 99/25840, and WO 99/25841; each of which is herein incorporated by reference in its entirety.

In particular embodiments, the expression cassette has the sequence set forth in SEQ ID NO: 45 or a variant or fragment thereof.

The expression cassette can be part of a vector that comprises multiple expression cassettes or multiple genes, such as a selectable marker gene. Selectable marker genes may be used to identify transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting. Any selectable marker gene can be used.

In some embodiments, an expression cassette comprising a presently disclosed promoter construct can further comprise a polynucleotide encoding a cell proliferation factor. As used herein, a “cell proliferation factor” is a polypeptide or a polynucleotide capable of stimulating growth of a cell or tissue, including but not limited to promoting progression through the cell cycle, inhibiting cell death, such as apoptosis, stimulating cell division, and/or stimulating embryogenesis. The polynucleotides can fall into several categories, including but not limited to, cell cycle stimulatory polynucleotides, developmental polynucleotides, anti-apoptosis polynucleotides, hormone polynucleotides, or silencing constructs targeted against cell cycle repressors or pro-apoptotic factors. The following are provided as non-limiting examples of each category and are not considered a complete list of useful polynucleotides for each category: 1) cell cycle stimulatory polynucleotides including plant viral replicase genes such as RepA, cyclins, E2F, prolifera, cdc2 and cdc25; 2) developmental polynucleotides such as Lec1, Kn1 family, WUSCHEL, Zwille, BBM, Aintegumenta (ANT), FUS3, and members of the Knotted family, such as Kn1, STM, OSH1, and SbHl; 3) anti-apoptosis polynucleotides such as CED9, Bcl2, Bcl-X(L), Bcl-W, A1, McL-1, Mac1, Boo, and Bax-inhibitors; 4) hormone polynucleotides such as IPT, TZS, and CKI-1; and 5) silencing constructs targeted against cell cycle repressors, such as Rb, CK1, prohibitin, and weel, or stimulators of apoptosis such as APAF-1, bad, bax, CED-4, and caspase-3, and repressors of plant developmental transitions, such as Pickle and WD polycomb genes including FIE and Medea. The polynucleotides can be silenced by any known method such as antisense, RNA interference, cosuppression, chimerplasty, or transposon insertion.

The cell proliferation factors can be introduced into cells through the introduction of a polynucleotide that encodes the proliferation factor. The use of the term “polynucleotide” is not intended to limit compositions to polynucleotides comprising DNA. Polynucleotides can comprise ribonucleotides and combinations of ribonucleotides and deoxyribonucleotides. Such deoxyribonucleotides and ribonucleotides include both naturally occurring molecules and synthetic analogues. The polynucleotides also encompass all forms of sequences including, but not limited to, single-, double-, or multi-stranded forms, hairpins, stem-and-loop structures, circular plasmids, and the like. The polynucleotide encoding the cell proliferation factor may be native to the cell or heterologous. A native polypeptide or polynucleotide comprises a naturally occurring amino acid sequence or nucleotide sequence. “Heterologous” in reference to a polypeptide or a nucleotide sequence is a polypeptide or a sequence that originates from a different species, or if from the same species, is substantially modified from its native form in composition and/or genomic locus by deliberate human intervention.

An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein or biologically active portion thereof is recombinantly produced, optimally culture medium represents less than about 30%, 20%, 10%, 5%, or 1% (by dry weight) of chemical precursors or non-protein-of-interest chemicals.

Any of a number of cell proliferation factors can be used. In certain embodiments, those cell proliferation factors that are capable of stimulating embryogenesis are used to enhance targeted polynucleotide modification. Such cell proliferation factors are referred to herein as embryogenesis-stimulating polypeptides and they include, but are not limited to, babyboom polypeptides.

In some embodiments, the cell proliferation factor is a member of the AP2/ERF family of proteins. The AP2/ERF family of proteins is a plant-specific class of putative transcription factors that regulate a wide variety of developmental processes and are characterized by the presence of an AP2 DNA binding domain that is predicted to form an amphipathic alpha helix that binds DNA (PFAM Accession PF00847). The AP2 domain was first identified in APETALA2, an Arabidopsis protein that regulates meristem identity, floral organ specification, seed coat development, and floral homeotic gene expression. The AP2/ERF proteins have been subdivided into distinct subfamilies based on the presence of conserved domains. Initially, the family was divided into two subfamilies based on the number of DNA binding domains, with the ERF subfamily having one DNA binding domain, and the AP2 subfamily having 2 DNA binding domains. As more sequences were identified, the family was subsequently subdivided into five subfamilies: AP2, DREB, ERF, RAV, and others. (Sakuma et al. (2002) Biochem Biophys Res Comm 290:998-1009).

Members of the APETALA2 (AP2) family of proteins function in a variety of biological events, including but not limited to, development, plant regeneration, cell division, embryogenesis, and cell proliferation (see, e.g., Riechmann and Meyerowitz (1998) Biol Chem 379:633-646; Saleh and Pages (2003) Genetika 35:37-50 and Database of Arabidopsis Transciption Factors at daft.cbi.pku.edu.cn). The AP2 family includes, but is not limited to, AP2, ANT, Glossy15, AtBBM, BnBBM, and maize ODP2/BBM.

Provided herein is an analysis of fifty sequences with homology to a maize BBM sequence (also referred to as maize ODP2 or ZmODP2, the polynucleotide and amino acid sequence of the maize BBM is set forth in SEQ ID NO: 9 and 10, respectively; the polynucleotide and amino acid sequence of another ZmBBM is set forth in SEQ ID NO: 121 and 122, respectively). The analysis identified three motifs (motifs 4-6; set forth in SEQ ID NOs: 51-53), along with the AP2 domains (motifs 2 and 3; SEQ ID NOs: 49 and 50) and linker sequence that bridges the AP2 domains (motif 1; SEQ ID NO: 48), that are found in all of the BBM homologues. Thus, motifs 1-6 distinguish these BBM homologues from other AP2-domain containing proteins (e.g., WRI, AP2, and RAP2.7) and these BBM homologues comprise a subgroup of AP2 family of proteins referred to herein as the BBM/PLT subgroup. In some embodiments, the cell proliferation factor that is used in the methods and compositions is a member of the BBM/PLT group of AP2 domain-containing polypeptides. In these embodiments, the cell proliferation factor comprises two AP2 domains and motifs 4-6 (SEQ ID NOs: 51-53) or a fragment or variant thereof. In some of these embodiments, the AP2 domains have the sequence set forth in SEQ ID NOs: 49 and 50 or a fragment or variant thereof, and in particular embodiments, further comprises the linker sequence of SEQ ID NO: 48 or a fragment or variant thereof. In other embodiments, the cell proliferation factor comprises at least one of motifs 4-6 or a fragment or variant thereof, along with two AP2 domains, which in some embodiments have the sequence set forth in SEQ ID NO: 49 and/or 50 or a fragment or variant thereof, and in particular embodiments have the linker sequence of SEQ ID NO: 48 or a fragment or variant thereof. Based on the phylogenetic analysis provided herein, the subgroup of BBM/PLT polypeptides can be subdivided into the BBM, AIL6/7, PLT1/2, AIL1, PLT3, and ANT groups of polypeptides.

In some embodiments, the cell proliferation factor is a babyboom (BBM) polypeptide, which is a member of the AP2 family of transcription factors. The BBM protein from Arabidopsis (AtBBM) is preferentially expressed in the developing embryo and seeds and has been shown to play a central role in regulating embryo-specific pathways. Overexpression of AtBBM has been shown to induce spontaneous formation of somatic embryos and cotyledon-like structures on seedlings. See, Boutiler et al. (2002) The Plant Cell 14:1737-1749. The maize BBM protein also induces embryogenesis and promotes transformation (See, U.S. Pat. No. 7,579,529, which is herein incorporated by reference in its entirety). Thus, BBM polypeptides stimulate proliferation, induce embryogenesis, enhance the regenerative capacity of a plant, enhance transformation, and as demonstrated herein, enhance rates of targeted polynucleotide modification. As used herein “regeneration” refers to a morphogenic response that results in the production of new tissues, organs, embryos, whole plants or parts of whole plants that are derived from a single cell or a group of cells. Regeneration may proceed indirectly via a callus phase or directly, without an intervening callus phase. “Regenerative capacity” refers to the ability of a plant cell to undergo regeneration.

In some embodiments, the babyboom polypeptide comprises two AP2 domains and at least one of motifs 7 and 10 (set forth in SEQ ID NO: 54 and 57, respectively) or a variant or fragment thereof. In certain embodiments, the AP2 domains are motifs 3 and 2 (SEQ ID NOs: 50 and 49, respectively) or a fragment or variant thereof, and in particular embodiments, the babyboom polypeptide further comprises a linker sequence between AP2 domain 1 and 2 having motif 1 (SEQ ID NO: 48) or a fragment or variant thereof. In particular embodiments, the BBM polypeptide further comprises motifs 4-6 (SEQ ID NOs 51-53) or a fragment or variant thereof. The BBM polypeptide can further comprise motifs 8 and 9 (SEQ ID NOs: 55 and 56, respectively) or a fragment or variant thereof, and in some embodiments, motif 10 (SEQ ID NO: 57) or a variant or fragment thereof. In some of these embodiments, the BBM polypeptide also comprises at least one of motif 14 (set forth in SEQ ID NO: 58), motif 15 (set forth in SEQ ID NO: 59), and motif 19 (set forth in SEQ ID NO: 60), or variants or fragments thereof. The variant of a particular amino acid motif can be an amino acid sequence having at least about 40%, 50%, 60%, 70%, 75%, 80%, 85%, 90%, 95%, 96%, 97%, 98%, 99%, or greater sequence identity with the motif disclosed herein. Alternatively, variants of a particular amino acid motif can be an amino acid sequence that differs from the amino acid motif by 1, 2, 3, 4, 5, 6, 7, 8, 9, or 10 amino acids.

Non-limiting examples of babyboom polynucleotides and polypeptides that can be used in the methods and compositions include the Arabidopsis thaliana AtBBM (SEQ ID NOs: 21 and 22), Brassica napus BnBBM1 (SEQ ID NOs: 23 and 24), Brassica napus BnBBM2 (SEQ ID NOs: 25 and 26), Medicago truncatula MtBBM (SEQ ID NOs: 7 and 8), Glycine max GmBBM (SEQ ID NOs: 1 and 2), Vitis vinifera VvBBM (SEQ ID NOs: 5 and 6), Zea mays ZmBBM (SEQ ID NOs: 9 and 10 and genomic sequence set forth in SEQ ID NO: 68; or SEQ ID NOs: 121 and 122 and genomic sequence set forth in SEQ ID NO: 116) and ZmBBM2 (SEQ ID NOs: 11 and 12), Oryza sativa OsBBM (polynucleotide sequences set forth in SEQ ID NOs: 13 and 120; amino acid sequence set forth in SEQ ID NO: 14; and genomic sequence set forth in SEQ ID NO: 117), OsBBM1 (SEQ ID NOs: 15 and 16), OsBBM2 (SEQ ID NOs: 17 and 18), and OsBBM3 (SEQ ID NOs: 19 and 20), Sorghum bicolor SbBBM (SEQ ID NOs: 3 and 4 and genomic sequence set forth in SEQ ID NO: 69) and SbBBM2 (SEQ ID NOs: 27 and 28) or active fragments or variants thereof. In particular embodiments, the cell proliferation factor is a maize BBM polypeptide (SEQ ID NO: 10, 122, or 12) or a variant or fragment thereof, or is encoded by a maize BBM polynucleotide (SEQ ID NO: 9, 68, 121, 116, or 11) or a variant or fragment thereof.

Thus, in some embodiments, a polynucleotide encoding a cell proliferation factor has a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the nucleotide sequence set forth in SEQ ID NO: 1, 3, 5, 7, 9, 11, 13, 15, 17, 19, 21, 23, 25, 27, 68, 116, 117, 120, 121, or 69 or the cell proliferation factor has an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 2, 4, 6, 8, 10, 12, 14, 16, 18, 20, 22, 24, 26, 122, or 28. In some of these embodiments, the cell proliferation factor has at least one of motifs 7 and 10 (SEQ ID NO: 54 and 57, respectively) or a variant or fragment thereof at the corresponding amino acid residue positions in the babyboom polypeptide. In other embodiments, the cell proliferation factor further comprises at least one of motif 14 (set forth in SEQ ID NO: 58), motif 15 (set forth in SEQ ID NO: 59), and motif 19 (set forth in SEQ ID NO: 60) or a variant or fragment thereof at the corresponding amino acid residue positions in the babyboom polypeptide.

In other embodiments, other cell proliferation factors, such as, Lec1, Kn1 family, WUSCHEL (e.g., WUS1, the polynucleotide and amino acid sequence of which is set forth in SEQ ID NO: 61 and 62; WUS2, the polynucleotide and amino acid sequence of which is set forth in SEQ ID NO: 63 and 64; WUS2 alt, the polynucleotide and amino acid sequence of which is set forth in SEQ ID NO: 114 and 115; WUS3, the polynucleotide and amino acid sequence of which is set forth in SEQ ID NO: 105 and 106), Zwille, and Aintegumeta (ANT), may be used alone, or in combination with a babyboom polypeptide or other cell proliferation factor. See, for example, U.S. Application Publication No. 2003/0135889, International Application Publication No. WO 03/001902, and U.S. Pat. No. 6,512,165, each of which is herein incorporated by reference. When multiple cell proliferation factors are used, or when a babyboom polypeptide is used along with any of the abovementioned polypeptides, the polynucleotides encoding each of the factors can be present on the same expression cassette or on separate expression cassettes. When two or more factors are coded for by separate expression cassettes, the expression cassettes can be provided to the plant simultaneously or sequentially.

In some embodiments, polynucleotides or polypeptides having homology to a known babyboom polynucleotide or polypeptide and/or sharing conserved functional domains can be identified by screening sequence databases using programs such as BLAST. The databases can be queried using full length sequences, or with fragments including, but not limited to, conserved domains or motifs. In some embodiments, the sequences retrieved from the search can be further characterized by alignment programs to quickly identify and compare conserved functional domains, regions of highest homology, and nucleotide and/or amino differences between sequences, including insertions, deletions, or substitutions, including those programs described in more detail elsewhere herein. The retrieved sequences can also be evaluated using a computer program to analyze and output the phylogenetic relationship between the sequences.

In other embodiments, polynucleotides or polypeptides having homology to a known babyboom polynucleotide or polypeptide or one that has been disclosed herein and/or sharing conserved functional domains can be identified using standard nucleic acid hybridization techniques, such as those described in more detail elsewhere herein. Extensive guides on nucleic acid hybridization include Tijssen (1993) Laboratory Techniques in Biochemistry and Molecular Biology—Hybridization with Nucleic Acid Probes, Part I, Chapter 2 (Elsevier, NY); Ausubel et al., eds. (1995) Current Protocols in Molecular Biology, Chapter 2 (Greene Publishing and Wiley-Interscience, NY); and, Sambrook et al. (1989) Molecular Cloning: A Laboratory Manual (2d ed., Cold Spring Harbor Laboratory Press, Plainview, NY).

Compositions further comprise isolated BBM polynucleotides and isolated BBM polypeptides and variants and fragments thereof, expression cassettes comprising the same, and plants comprising the same. Compositions can comprise isolated polynucleotides encoding GmBBM (SEQ ID NO: 1), SbBBM (SEQ ID NO: 3), MtBBM (SEQ ID NO: 7), or OsBBM2 (SEQ ID NO: 17) or an active variant or fragment thereof. Isolated polypeptides include those having SEQ ID NO: 2, 4, 8, or 18 (GmBBM, SbBBM, MtBBM, or OsBBM2, respectively) or an active variant or fragment thereof. The percent identity of the novel BBM polypeptide sequences with those known in the art is presented in Table 1.

TABLE 1
The percent sequence identity between each of 14 babyboom polypeptides.

| | Zm | Sb | Os | Os | Os | Zm | Sb | Os | Bn | Bn | At | Mt | Gm | Vv |
|---|---|---|---|---|---|---|---|---|---|---|---|---|---|---|
| | BBM2 | BBM2 | BBM2 | BBM3 | BBM1 | BBM | BBM | BBM | BBM1 | BBM2 | BBM | BBM | BBM | BBM |
| ZmBBM2 | 100 | | | | | | | | | | | | | |
| SbBBM2 | 92 | 100 | | | | | | | | | | | | |
| OsBBM2 | 79 | 77 | 100 | | | | | | | | | | | |
| OsBBM3 | 64 | 66 | 67 | 100 | | | | | | | | | | |
| OsBBM1 | 50 | 46 | 46 | 46 | 100 | | | | | | | | | |
| ZmBBM | 43 | 44 | 44 | 47 | 47 | 100 | | | | | | | | |
| SbBBM | 43 | 44 | 42 | 45 | 44 | 90 | 100 | | | | | | | |
| OsBBM | 44 | 44 | 45 | 46 | 49 | 69 | 70 | 100 | | | | | | |
| BnBBM1 | 42 | 41 | 42 | 41 | 40 | 45 | 42 | 43 | 100 | | | | | |
| BnBBM2 | 43 | 41 | 42 | 41 | 39 | 46 | 44 | 44 | 97 | 100 | | | | |
| AtBBM | 43 | 41 | 39 | 42 | 41 | 43 | 43 | 42 | 81 | 82 | 100 | | | |
| MtBBM | 41 | 40 | 40 | 41 | 43 | 42 | 43 | 41 | 47 | 47 | 47 | 100 | | |
| GmBBM | 45 | 44 | 42 | 45 | 44 | 41 | 42 | 44 | 46 | 46 | 43 | 68 | 100 | |
| VvBBM | 51 | 48 | 50 | 48 | 50 | 48 | 47 | 48 | 49 | 49 | 48 | 58 | 62 | 100 |

By “fragment” is intended a portion of the polynucleotide or a portion of an amino acid sequence and hence protein encoded thereby. Fragments of a polynucleotide may retain the biological activity of the native polynucleotide and, for example, have promoter activity (i.e., capable of initiating transcription), or are capable of stimulating proliferation, inducing embryogenesis, or modifying the regenerative capacity of a plant. In those embodiments wherein the polynucleotide encodes a polypeptide, fragments of the polynucleotide may encode protein fragments that retain the biological activity of the native protein. Alternatively, fragments of a polynucleotide that are useful as hybridization probes generally do not retain biological activity or encode fragment proteins that retain biological activity. Thus, fragments of a nucleotide sequence may range from at least about 20, 50, 100, 150, 200, 250, 300, 400, 500 nucleotides, or greater.

A fragment of a polynucleotide that encodes a biologically active portion of a cell proliferation factor, for example, will encode at least 15, 25, 30, 50, 100, 150, 200, 250, 300, 400, 500 contiguous amino acids, or up to the total number of amino acids present in the full-length cell proliferation factor. Fragments of a cell proliferation factor polynucleotide that are useful as hybridization probes or PCR primers generally need not encode a biologically active portion of a cell proliferation factor.

“Variants” is intended to mean substantially similar sequences. For polynucleotides, a variant comprises a polynucleotide having deletions at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides encoding polypeptides conservative variants include those sequences that, because of the degeneracy of the genetic code, encode the amino acid sequence the polypeptide (e.g., cell proliferation factor). Naturally occurring variants such as these can be identified with the use of well-known molecular biology techniques, such as, for example, with polymerase chain reaction (PCR) and hybridization techniques. Variant polynucleotides also include synthetically derived polynucleotides, such as those generated, for example, by using site-directed mutagenesis. Generally, variants of a particular will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters.

Variants of a particular polynucleotide that encodes a polypeptide can also be evaluated by comparison of the percent sequence identity between the polypeptide encoded by a variant polynucleotide and the polypeptide encoded by the particular polynucleotide. Percent sequence identity between any two polypeptides can be calculated using sequence alignment programs and parameters. Where any given pair of polynucleotides is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion of one or more amino acids at the N-terminal and/or C-terminal end of the native protein; deletion and/or addition of one or more amino acids at one or more internal sites in the native protein; and/or substitution of one or more amino acids at one or more sites in the native protein. Variant proteins retain the desired biological activity of the native protein. For example, variant cell proliferation factors stimulate proliferation and variant babyboom polypeptides are capable of stimulating proliferation, inducing embryogenesis, modifying the regenerative capacity of a plant, increasing the transformation efficiency in a plant, increasing or maintaining the yield in a plant under abiotic stress, producing asexually derived embryos in a plant, and/or enhancing rates of targeted polynucleotide modification. Such variants may result from, for example, genetic polymorphism or from human manipulation. Biologically active variants of a native cell proliferation factor will have at least about 40%, 45%, 50%, 55%, 60%, 6%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters. A biologically active variant of a cell proliferation factor protein may differ from that protein by as few as 1-15 amino acid residues, as few as 1-10, such as 6-10, as few as 5, as few as 4, 3, 2, or even 1 amino acid residue.

In some embodiments, variants or fragments of the BBM polypeptide have amino acid residues valine, tyrosine, and leucine at the positions corresponding to positions 311, 312, and 313, respectively, of SEQ ID NO: 4 or variants or fragments of the BBM polynucleotide encodes a polypeptide having amino acid residues valine, tyrosine, and leucine at the positions corresponding to positions 311, 312, and 313, respectively, of SEQ ID NO: 4. In certain embodiments, variants or fragments of the BBM polypeptide have amino acid residues valine, tyrosine, and leucine at the positions corresponding to positions 337, 338, and 339, respectively, of SEQ ID NO: 18 or variants or fragments of the BBM polynucleotide encodes a polypeptide having amino acid residues valine, tyrosine, and leucine at the positions corresponding to positions 337, 338, and 339, respectively, of SEQ ID NO: 18. In other embodiments, variants or fragments of the BBM polypeptide have amino acid residues methionine, alanine, and serine at the positions corresponding to positions 1, 2, and 3, respectively, of SEQ ID NO: 8 or variants or fragments of the BBM polynucleotide encodes a polypeptide having amino acid residues methionine, alanine, and serine at the positions corresponding to positions 1, 2, and 3, respectively of SEQ ID NO: 8.

The babyboom polynucleotides and polypeptides can be introduced into a plant or plant cell in order to stimulate embryogenesis, modify the regenerative capacity of the plant, increase the transformation efficiency of the plant, increase or maintain the yield in the plant under abiotic stress, and/or to enhance targeted polynucleotide modification. The babyboom polynucleotide or polypeptide can be provided to a plant simultaneously with or prior to the introduction of a polynucleotide of interest in order to facilitate transformation of the plant with the polynucleotide of interest. Further, a haploid plant cell can be provided a novel babyboom polynucleotide or polypeptide to produce a haploid plant embryo (see U.S. Pat. No. 7,579,529, which is herein incorporated by reference in its entirety).

The cell proliferation factor polynucleotide can be operably linked to a promoter active in a plant. Various promoters can be used for the regulation of the expression of the cell proliferation factor. The promoter may be selected based on the desired outcome or expression pattern (for a review of plant promoters, see Potenza et al. (2004) In Vitro Cell Dev Biol 40:1-22).

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), the Agrobacterium nopaline synthase (NOS) promoter (Bevan et al. (1983) Nucl. Acids Res. 11:369-385), and the like. Other constitutive promoters are described in, for example, U.S. Pat. Nos. 5,608,149; 5,608,144; 5,604,121; 5,569,597; 5,466,785; 5,399,680; 5,268,463; 5,608,142; and 6,177,611.

In some embodiments, an inducible promoter can be used, such as from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference. Promoters that are expressed locally at or near the site of pathogen infection include, for example, Marineau et al. (1987) Plant Mol. Biol. 9:335-342; Matton et al. (1989) Mol Plant-Microbe Interact 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Additional promoters include the inducible promoter for the maize PRms gene, whose expression is induced by the pathogen Fusarium moniliforme (see, for example, Cordero et al. (1992) Physiol. Mol. Plant Path. 41:189-200). Wound-inducible promoters include potato proteinase inhibitor (pin II) gene (Ryan (1990) Ann. Rev. Phytopath. 28:425-449; Duan et al. (1996) Nat Biotechnol 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Lett 323:73-76); MPI gene (Corderok et al. (1994) Plant J. 6:141-150); and the like, herein incorporated by reference. Another inducible promoter is the maize In2-2 promoter (deVeylder et al. (2007) Plant Cell Physiol 38:568-577, herein incorporated by reference).

Chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners (De Veylder et al. (1997) Plant Cell Physiol. 38:568-77), the maize GST promoter (GST-II-27, WO 93/01294), which is activated by hydrophobic electrophilic compounds that are used as pre-emergent herbicides, the PR-1 promoter (Cao et al. (2006) Plant Cell Reports 6:554-60), which is activated by BTH or benxo(1,2,3)thiaidazole-7-carbothioic acid s-methyl ester, the tobacco PR-la promoter (Ono et al. (2004) Biosci. Biotechnol. Biochem. 68:803-7), which is activated by salicylic acid, the copper inducible ACE1 promoter (Mett et al. (1993) PNAS 90:4567-4571), the ethanol-inducible promoter AlcA (Caddick et al. (1988) Nature Biotechnol 16:177-80), an estradiol-inducible promoter (Bruce et al. (2000) Plant Cell 12:65-79), the XVE estradiol-inducible promoter (Zao et al. (2000) Plant J 24:265-273), the VGE methoxyfenozide inducible promoter (Padidam et al. (2003) Transgenic Res 12:101-109), and the TGV dexamethasone-inducible promoter (Bohner et al. (1999) Plant J 19:87-95). Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237; Gatz et al. (1992) Plant J 2:397-404; and U.S. Pat. Nos. 5,814,618 and 5,789,156), herein incorporated by reference.

Tissue-preferred promoters can be utilized to target enhanced expression of a sequence of interest within a particular plant tissue. Tissue-preferred promoters include Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Lam (1994) Results Probl. Cell Differ. 20:181-196; and Guevara-Garcia et al. (1993) Plant J. 4(3):495-505.

Leaf-preferred promoters are known in the art. See, for example, Yamamoto et al. (1997) Plant J. 12:255-265; Kwon et al. (1994) Plant Physiol. 105:357-67; Yamamoto et al. (1994) Plant Cell Physiol. 35:773-778; Gotor et al. (1993) Plant J. 3:509-18; Orozco et al. (1993) Plant Mol. Biol. 23:1129-1138; and Matsuoka et al. (1993) Proc. Natl. Acad. Sci. USA 90:9586-9590. In addition, promoter of cab and rubisco can also be used. See, for example, Simpson et al. (1958) EMBO J 4:2723-2729 and Timko et al. (1988) Nature 318:57-58.

Root-preferred promoters are known and can be selected from the many available. See, for example, Hire et al. (1992) Plant Mol. Biol. 20:207-218 (soybean root-specific glutamine synthase gene); Keller and Baumgartner (1991) Plant Cell 3:1051-1061 (root-specific control element in the GRP 1.8 gene of French bean); Sanger et al. (1990) Plant Mol. Biol. 14:433-443 (root-specific promoter of the mannopine synthase (MAS) gene of Agrobacterium tumefaciens); and Miao et al. (1991) Plant Cell 3:11-22 (full-length cDNA clone encoding cytosolic glutamine synthase (GS), which is expressed in roots and root nodules of soybean). See also Bogusz et al. (1990) Plant Cell 2:633-641, where two root-specific promoters isolated from hemoglobin genes from the nitrogen-fixing nonlegume Parasponia andersonii and the related non-nitrogen-fixing nonlegume Trema tomentosa are described. Leach and Aoyagi (1991) describe their analysis of the promoters of the highly expressed rolC and rolD root-inducing genes of Agrobacterium rhizogenes (see Plant Sci (Limerick) 79:69-76). Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue (see EMBO J. 8:343-350). The TR1′ gene, fused to nptI (neomycin phosphotransferase II) showed similar characteristics. Additional root-preferred promoters include the VfENOD-GRP3 gene promoter (Kuster et al. (1995) Plant Mol. Biol. 29:759-772); and rolB promoter (Capana et al. (1994) Plant Mol. Biol. 25:681-691. See also U.S. Pat. Nos. 5,837,876; 5,750,386; 5,633,363; 5,459,252; 5,401,836; 5,110,732; and 5,023,179. Another root-preferred promoter includes the promoter of the phaseolin gene (Murai et al. (1983) Science 23:476-482 and Sengopta-Gopalen et al. (1988) Proc. Natl. Acad. Sci. USA 82:3320-3324.

Seed-preferred promoters include both those promoters active during seed development as well as promoters active during seed germination. See Thompson et al. (1989) BioEssays 10:108, herein incorporated by reference. Such seed-preferred promoters include, but are not limited to, Cim1 (cytokinin-induced message); cZ19B1 (maize 19 kDa zein); and milps (myo-inositol-1-phosphate synthase); (see WO 00/11177 and U.S. Pat. No. 6,225,529; herein incorporated by reference). For dicots, seed-preferred promoters include, but are not limited to, bean 0-phaseolin, napin, 0-conglycinin, soybean lectin, cruciferin, and the like. For monocots, seed-preferred promoters include, but are not limited to, maize 15 kDa zein, 22 kDa zein, 27 kDa gamma zein, waxy, shrunken 1, shrunken 2, globulin 1, oleosin, nuc1, etc. See also WO 00/12733, where seed-preferred promoters from end1 and end2 genes are disclosed; herein incorporated by reference.

Where low-level expression is desired, weak promoters will be used. Generally, by “weak promoter” is intended a promoter that drives expression of a coding sequence at a low level. By low level is intended at levels of about 1/1000 transcripts to about 1/100,000 transcripts to about 1/500,000 transcripts. Alternatively, it is recognized that weak promoters also encompasses promoters that are expressed in only a few cells and not in others to give a total low level of expression. Where a promoter is expressed at unacceptably high levels, portions of the promoter sequence can be deleted or modified to decrease expression levels. Such weak constitutive promoters include, for example, the core promoter of the Rsyn7 promoter (WO 99/43838 and U.S. Pat. No. 6,072,050), the core 35S CaMV promoter, and the like.

Other promoters of interest include the Rab16 promoter (Mundy et al. (1990) PNAS 87: 1406-1410), the Brassica LEA3-1 promoter (U.S. Application Publication No. US 2008/0244793), the HVA1s, Dhn8s, and Dhn4s from barley and the wsi18j, rab16Bj from rice (Xiao and Xue (2001) Plant Cell Rep 20:667-73), and D 113 from cotton (Luo et al. (2008) Plant Cell Rep 27:707-717).

In some embodiments, the polynucleotide encoding a cell proliferation factor (e.g., babyboom polypeptide) is operably linked to a maize ubiquitin promoter or a maize oleosin promoter (e.g., SEQ ID NO: 65 or a variant or fragment thereof).

In some of those embodiments wherein the vector comprises a presently disclosed promoter construct operably linked to a polynucleotide encoding a site-specific recombinase and in some embodiments, a polynucleotide encoding a babyboom polypeptide, the vector can further comprise a polynucleotide encoding a Wuschel polypeptide (see International Application Publication No. WO 01/23575 and U.S. Pat. No. 7,256,322, each of which are herein incorporated by reference in its entirety). In certain embodiments, the polynucleotide encoding the Wuschel polypeptide has the sequence set forth in SEQ ID NO: 61, 63, 114, or 105 (WUS1, WUS2, WUS2 alt, or WUS3, respectively) or an active variant or fragment thereof. In particular embodiments, the Wuschel polypeptide has the sequence set forth in SEQ ID NO: 62, 64, 115, or 106 (WUS1, WUS2, WUS2 alt, or WUS3, respectively) or an active variant or fragment thereof. In some of these embodiments, the polynucleotide encoding a Wuschel polypeptide is operably linked to a promoter active in the plant, including but not limited to the maize In2-2 promoter or a nopaline synthase promoter. In some of these embodiments, the expression cassettes for the site-specific recombinase, the babyboom polypeptide, and the Wuschel polypeptide are all flanked by site-specific recombination sites that are directly repeated and are recognized by the site-specific recombinase whose expression is regulated by a presently disclosed promoter construct, such that expression of the site-specific recombinase results in the excision of the three expression cassettes.

In some embodiments, the vector comprises a promoter disclosed herein (maize Rab17 promoter with an attB1 site) operably linked to a site-specific recombinase (e.g., Cre, FLP); a second promoter operably linked to a cell proliferation factor (e.g., a babyboom polypeptide); and a third promoter operably linked to a polynucleotide of interest, such as those disclosed elsewhere herein (e.g., trait gene), or multiple polynucleotides of interest operably linked to one or more promoters; and in some embodiments, a fourth promoter operably linked to a WUS gene. In some of these embodiments, the expression cassettes for the site-specific recombinase, the cell proliferation factor, and the Wuschel polypeptide are all flanked by site-specific recombination sites that are directly repeated and are recognized by the site-specific recombinase, such that expression of the site-specific recombinase results in the excision of the three expression cassettes, leaving the polynucleotides of interest (e.g., trait genes) behind. In other embodiments, the polynucleotide of interest (e.g., trait gene) is introduced along with or following the vector comprising a presently disclosed promoter operably linked to a site-specific recombinase, and at least one cell proliferation factor (e.g., babyboom polypeptide, Wuschel polypeptide) operably linked to one or more promoters, wherein the polynucleotide of interest is present on a separate vector from the expression cassettes for the site-specific recombinase and cell proliferation factor(s). In some of these embodiments, the expression cassettes for the site-specific recombinase and cell proliferation factor(s) are flanked by recombination sites that are recognized by the site-specific recombinase. Expression of the cell proliferation factors facilitates the transformation of the polynucleotide of interest (e.g., trait gene) and expression of the site-specific recombinase results in the excision of the expression cassettes for the site-specific recombinase and cell proliferation factor(s).

The presently disclosed promoter constructs, expression cassettes, and vectors can be introduced into a host cell. By “host cell” is meant a cell, which comprises a heterologous nucleic acid sequence. Host cells may be prokaryotic cells such as E. coli, or eukaryotic cells such as yeast, insect, amphibian, or mammalian cells. In some examples, host cells are monocotyledonous or dicotyledonous plant cells. In particular embodiments, the monocotyledonous host cell is a maize host cell.

An intermediate host cell may be used, for example, to increase the copy number of the cloning vector and/or to mediate transformation of a different host cell. With an increased copy number, the vector containing the nucleic acid of interest can be isolated in significant quantities for introduction into the desired plant cells. In one embodiment, plant promoters that do not cause expression of the polypeptide in bacteria are employed.

Prokaryotes most frequently are represented by various strains of E. coli; however, other microbial strains may also be used. Commonly used prokaryotic control sequences which are defined herein to include promoters for transcription initiation, optionally with an operator, along with ribosome binding sequences, include such commonly used promoters as the beta lactamase (penicillinase) and lactose (lac) promoter systems (Chang et al. (1977) Nature 198:1056), the tryptophan (trp) promoter system (Goeddel et al. (1980) Nucleic Acids Res. 8:4057) and the lambda derived P L promoter and N-gene ribosome binding site (Shimatake et al. (1981) Nature 292:128). The inclusion of selection markers in DNA vectors transfected in E. coli is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein are available using Bacillus sp. and Salmonella (Palva et al. (1983) Gene 22:229-235); Mosbach et al. (1983) Nature 302:543-545).

Methods for expressing a polynucleotide of interest in a plant comprise introducing an expression cassette or vector. Alternatively, the method can comprise introducing a promoter construct, wherein the promoter construct is stably integrated into the genome of the plant and operably linked to a polynucleotide of interest.

“Introducing” is intended to mean presenting to the organism, such as a plant, or the cell the polynucleotide or polypeptide in such a manner that the sequence gains access to the interior of a cell of the organism or to the cell itself. The methods and compositions do not depend on a particular method for introducing a sequence into an organism or cell, only that the polynucleotide or polypeptide gains access to the interior of at least one cell of the organism. Methods for introducing polynucleotides or polypeptides into plants are known in the art including, but not limited to, stable transformation methods, transient transformation methods, virus-mediated methods, and sexual breeding.

“Stable transformation” is intended to mean that the nucleotide construct introduced into a plant integrates into a genome of the plant and is capable of being inherited by the progeny thereof. “Transient transformation” is intended to mean that a polynucleotide is introduced into the plant and does not integrate into a genome of the plant or a polypeptide is introduced into a plant.

Protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Rep 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Rep 12:250-255 and Christou and Ford (1995) Annals of Botany 75:407-413 (rice); Osjoda et al. (1996) Nat Biotechnol 14:745-750 (maize via Agrobacterium tumefaciens); all of which are herein incorporated by reference.

In specific embodiments, the sequences can be provided to a plant using a variety of transient transformation methods. Such transient transformation methods include, but are not limited to, the introduction of the polypeptide of interest directly into the plant or the introduction of a polynucleotide encoding the polypeptide of interest into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91:2176-2180 and Hush et al. (1994) J Cell Sci 107:775-784, all of which are herein incorporated by reference. Alternatively, the polynucleotide can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which its released to become integrated into the genome is greatly reduced. Such methods include the use particles coated with polyethylimine (PEI; Sigma #P3143).

In other embodiments, the polynucleotide may be introduced into plants by contacting plants with a virus or viral nucleic acids. Generally, such methods involve incorporating a nucleotide construct within a viral DNA or RNA molecule. It is recognized that the cell proliferation factor may be initially synthesized as part of a viral polyprotein, which later may be processed by proteolysis in vivo or in vitro to produce the desired recombinant protein. Further, it is recognized that promoters also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Other methods of introducing polynucleotides into a plant can be used, including plastid transformation methods, and the methods for introducing polynucleotides into tissues from seedlings or mature seeds.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide can be contained in a transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

In specific embodiments, methods are provided for the excision of a polynucleotide of interest from a target site in a plant, wherein the polynucleotide of interest is flanked by a first and a second recombination site that are recombinogenic with respect to one another and that are directly repeated. The method comprises introducing into the plant an expression cassette comprising a presently disclosed promoter construct (e.g., SEQ ID NO: 30 or a variant or fragment thereof) operably linked to a site-specific recombinase, expressing the recombinase, so that the recombinase recognizes and implements recombination at the recombination sites flanking the polynucleotide of interest, thereby excising the polynucleotide of interest. The expression cassette can comprise any of the linker sequences, attB sites, termination regions, etc., such as those described herein.

The terms “target site,” and “target sequence,” as used interchangeably herein, refer to a polynucleotide sequence present in a cell of an organism, such as a plant, that comprises at least one site-specific recombination site. The target site may be part of the organism's native genome or integrated therein or may be present on an episomal polynucleotide. The genomic target sequence may be on any region of any chromosome, and may or may not be in a region encoding a protein or RNA. The target site may be native to the cell or heterologous. In some embodiments, the heterologous target sequence may have been transgenically inserted into the organism's genome, and may be on any region of any chromosome, including an artificial or satellite chromosome, and may or may not be in a region encoding a protein or RNA. It is recognized that the cell or the organism may comprise multiple target sites, which may be located at one or multiple loci within or across chromosomes.

Alternative methods for excising a polynucleotide of interest from a target site in a plant include providing a plant comprising a target site comprising in operable linkage: a first site-specific recombination site, a first promoter, the polynucleotide of interest, a second promoter, a polynucleotide encoding a site-specific recombinase, and a second site-specific recombination site. The first and the second site-specific recombination sites are recombinogenic with respect to one another and directly repeated. The polynucleotide of interest and its operably linked promoter may precede or follow the polynucleotide encoding the site-specific recombinase and its operably linked promoter. The second promoter is one of the presently disclosed promoter constructs (e.g., SEQ ID NO: 30 or a variant or fragment thereof). The method comprises expressing the site-specific recombinase, whereby the site-specific recombinase recognizes and implements recombination at the first and the second site-specific recombination sites, thereby excising the polynucleotide of interest and the polynucleotide encoding the site-specific recombinase.

In some embodiments, the target site further comprises a third promoter operably linked to a polynucleotide encoding a Wuschel polypeptide. The three expression cassettes may be in any order, but in some embodiments, the target site comprises in operable linkage: the first site-specific recombination site, the third promoter, the polynucleotide encoding a Wuschel polypeptide, the first promoter, the polynucleotide of interest, the second promoter, the polynucleotide encoding the site-specific recombinase, and the second site-specific recombination site, wherein expression of the recombinase results in the excision of all three expression cassettes. The expression cassette can comprise any of the linker sequences, attB sites, termination regions, etc., such as those described herein.

Methods are provided to enhance the efficiency of plastid transformation, which include introducing into a plant cell a heterologous polynucleotide encoding a cell proliferation factor and expressing the heterologous polynucleotide before, during, or immediately following the transformation of the plastid of the plant cell with a polynucleotide of interest. The heterologous polynucleotide encoding a cell proliferation factor can be co-delivered with the polynucleotide of interest or the cell proliferation polynucleotide can first be introduced into the plant, followed by the introduction of the polynucleotide of interest.

As used herein, a “plastid” refers to an organelle present in plant cells that stores and manufactures chemical compounds used by the cell, such as starch, fatty acids, terpenes, and that has been derived from a proplastid. Thus, plastids of plants typically have the same genetic content. Plastids include chloroplasts, which are responsible for photosynthesis, amyloplasts, chromoplasts, statoliths, leucoplasts, elaioplasts, and proteinoplasts.

The plastid genome is circular and varies in size among plant species from about 120 to about 217 kilobase pairs (kb). The genome typically includes a large inverted repeat, which can contain up to about 76 kilobase pairs, but which is more typically in the range of about 20 to about 30 kilobase pairs. The inverted repeat present in the plastid genome of various organisms has been described (Palmer (1990) Trends Genet 6:115-120).

Transformation of plastids can result in a homoplasmic state, wherein essentially all of the plastids in a plant cell have the introduced DNA integrated into the plastid genome. This occurs through a selection process, whereby those cells that comprise a sufficient number of transformed plastids having an introduced selectable marker gene survive on the selection medium, and through the reproduction of the transformed plastid genomes. Plastids can be present in a plant cell at a very high copy number, with up to 50,000 copies per cell present for the chloroplast genome (Bendich (1987) BioEssays 6:279-282). Thus, through plastid transformation, plant cells can be engineered to maintain an introduced gene of interest at a very high copy number.

While plastid transformation is routine and relatively efficient in tobacco by bombardment of leaves, the application of plastid transformation technology in important crop species is not routine. For example, plastid transformation in maize and wheat has not been reported. Plastid transformation is possible in soybean, but the frequency of transformation with vectors carrying trait genes is low. Plastid transformation is possible in rice, but homoplasmic events have not been recovered.

The introduction and expression of polynucleotides encoding cell proliferation factors may be used to enhance the efficiency of plastid transformation. Any cell proliferation factor known in the art or described elsewhere herein may be used to enhance plastid transformation, including babyboom polypeptides. In certain embodiments, embryogenesis-stimulating polypeptides are used to enhance plastid transformation.

Methods are known in the art for introducing genes into the plastid genome. See, for example, Svab et al. (1990) Proc. Natl. Acad. Sci. USA 87: 8526-8530; Svab and Maliga (1993) Proc. Natl. Acad. Sci. USA 90: 913-917; Svab and Maliga (1993) EMBO J. 12: 601-606; and U.S. Pat. Nos. 5,451,513 and 5,545,818; each of which is herein incorporated by reference in its entirety.

One method involves the integration of a polynucleotide of interest into the plastid genome through homologous recombination. Such methods involve the introduction of a polynucleotide of interest flanked by regions of homology with regions of the plastid genome into a plant cell. Delivery of the polynucleotide of interest into the plant cell can be via any method of transformation known in the art, including those described elsewhere herein. These include, but are not limited to, particle gun delivery (Svab, Z. et al. (1990) Proc Natl Acad Sci USA 87:8526-8530; Svab and Maliga (1993) Proc Natl Acad Sci USA 90:913-917; and Staub and Maliga (1993) EMBO J 12:601-606; and U.S. Pat. Nos. 5,451,513 and 5,545,818; each of which is herein incorporated by reference in its entirety) and Agrobacterium-mediated transformation (U.S. Pat. Nos. 5,563,055 and 5,981,840). In some species, protoplasts can also be used for chloroplast transformation (O'Neill et al. (1993) Plant J 3:729-38; and Spoerlein et al. (1991) Theor Appl Gen 82:717-722; each of which is herein incorporated by reference in its entirety). Once the polynucleotide of interest flanked by the homologous regions enters the cell, the polynucleotide of interest will be integrated within the plastid genome.

The homologous regions flanking the polynucleotide of interest, and in some embodiments, its operably linked promoter, and in particular embodiments, the selectable marker gene as well, may vary in length. In some embodiments, the region of homology with the plastid genome is about 50, 75, 100, 200, 300, 400, 500, 600, 700, 800, 900, 1000 base pairs or greater in length. In most instances, the frequency of recombination and thus the frequency of obtaining plants having transformed plastids decreases with the decreasing size of the homologous regions. In those embodiments wherein the regions of homology are present in the inverted repeat regions of the plastid genome, two copies of the polynucleotide of interest are expected per transformed plastid.

In some embodiments, the polynucleotide of interest can be co-delivered with a selectable marker gene that is active in the plastid. The selectable marker gene and the polynucleotide of interest can be present on a single DNA construct or on separate constructs. A number of markers have been developed for use with plant cells, such as resistance to chloramphenicol, the aminoglycoside G418, hygromycin, or the like. Genes conferring resistance to kanamycin (NPTII or AphA6) have been used as a selectable marker for plastid transformation (Carrer et al. (1993) Mol Gen Genetics 241:49-56; and Huang et al. (2002) Mol Gen Genomics 268:19-27; each of which is herein incorporated by reference in its entirety). Other genes which encode a product involved in chloroplast metabolism may also be used as selectable markers.

Another example of a selectable marker gene for plastid transformation is a selectable marker gene that confers resistance to a substance which inhibits protein synthesis by the plastids, such that cells which have acquired the phenotype are selected for by contacting the cells with a substance which inhibits protein synthesis by the plastids. The plastid DNA encoding the nonlethal selectable phenotype may comprise 16S ribosomal DNA mutated to confer resistance to the effects of streptomycin, or to spectinomycin, or to both antibiotics simultaneously. Expression of heterologous genes that modify non-lethal antibiotics such as streptomycin or spectinomycin by phosphorylation, adenylation or acetylation also are suitable for the selection of plastid transformation events. Another non-limiting example of a gene that confers resistance to streptomycin and spectinomycin is the bacterial aadA gene that codes for streptomycin/spectinomycin adenyltransferase (Svab et al. (1993) Proc Natl Acad Sci USA 90:913-917). The aadA gene product allows for continued growth and greening of cells in the presence of streptomycin or spectinomycin whose chloroplasts comprise the selectable marker gene product. Cells which do not contain the selectable marker gene product are bleached. Selection for the aadA gene marker is thus based on identification of plant cells which are not bleached by the presence of streptomycin or spectinomycin, in the plant growth medium.

Other examples of selectable marker genes are those that confer resistance to an herbicide, including a photosystem II herbicide, such as a triazine herbicide, specifically the triazine herbicide atrazine. This phenotype not only provides nonlethal selection, but also provides herbicide resistance. Genes that provide resistance to plant herbicides such as glyphosate, bromoxynil, or imidazolinone may find use as a selectable marker gene. Such genes have been reported (Stalker et al. (1985) J Biol Chem 260:4724-4728 (glyphosate resistant EPSP); Stalker et al. (1985) J Biol Chem 263:6310-6314 (bromoxynil resistant nitrilase gene); and Sathasivan et al. (1990) Nucl Acids Res 18:2188 (AHAS imidazolinone resistance gene); each of which is herein incorporated by reference in its entirety).

The selectable marker gene and/or the polynucleotide of interest can be placed under the regulatory control of a chloroplast 5′ promoter and 3′ transcription termination regions, such as the tobacco 16S rRNA promoter rrn region and rps16 3′ termination region. Numerous additional promoter regions may also be used to drive expression of the selectable marker gene and/or the polynucleotide of interest, including various plastid promoters and bacterial promoters which have been shown to function in plant plastids. Further, if nuclear expression of the selectable marker gene and/or the polynucleotide of interest is not desired, plastid introns can be incorporated into the selectable marker gene and/or the polynucleotide of interest. Certain classes of plastid introns can not be correctly spliced out in the nucleus, thereby preventing expression of the selectable marker gene and/or the polynucleotide of interest within the nucleus. The polynucleotide of interest and/or the heterologous polynucleotide encoding the cell proliferation factor may be optimized for expression in the chloroplast to account for differences in codon usage between the plant nucleus and this organelle. In this manner, the polynucleotide may be synthesized using chloroplast-preferred codons. See, for example, U.S. Pat. No. 5,380,831, herein incorporated by reference.

An additional method of plastid transformation occurs through the transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91: 7301-7305, which is herein incorporated by reference in its entirety. In these methods, the heterologous polynucleotide encoding the cell proliferation factor is introduced into the cell and expressed prior to, during, or immediately after the expression of the plastid-directed RNA polymerase.

In order to select those cells having transformed plastids, following introduction of the chloroplast transformation vectors, the treated tissue is placed on tissue culture medium containing the appropriate selection agent. After a suitable period of incubation on selection medium, transformed cells can be identified and grown to a stage that allows regeneration of the whole plants. The regeneration processes are basically identical to those used for standard nuclear transformation events. Special care must be taken to ensure that selection and regeneration conditions promote the elimination of most wild-type chloroplast genomes. The status of the proportion of wild-type to transformed chloroplast genomes can be monitored by standard molecular techniques including Southern and PCR analysis.

For tobacco and a number of other species, leaves are a preferred target for plastid transformation. In some embodiments, one or more cell proliferation factors (e.g., babyboom polypeptides) can be used to trigger a tissue culture response from leaves of maize and other species. For boosting chloroplast transformation, polynucleotides encoding cell proliferation factors under the control of inducible promoters can be introduced into the species of interest by standard nuclear transformation protocols. Events that contain the transgene can be characterized for expression of the inducible embryogenesis-stimulating polypeptides. Then, the expression of the polynucleotide encoding the cell proliferation factor is induced, thereby stimulating an embryogenic tissue culture response. For example, leaves from plants transformed with the polynucleotide(s) encoding a cell proliferation factor under the control of the tetracycline-repressor system can be placed on medium containing appropriate concentrations of doxycyline for induction of expression. The leaves can be maintained on the induction medium to allow for cell division and the initiation of embryogenic callus to take place. The plastids of the leaves can be transformed with the polynucleotide of interest, and in certain embodiments, a selectable marker gene just prior to the induction of the polynucleotide(s) encoding cell proliferation factor, during induction, or immediately after induction. Alternatively, leaf tissue can be transformed using the methods disclosed elsewhere herein. After plastid transformation, the plastid transformation events can be selected by incubating the leaves on selection medium. Following selection, the leaves or plant cells are grown on medium that stimulates callus formation.

Methods are provided for the preparation and transformation of dried mature seeds, mature embryos, and mature embryo explants. A mature embryo explant is a tissue dissected from a mature embryo, which is an embryo that has an age of at least about 18 days after pollination. Methods for preparing a mature embryo comprise dissecting a mature embryo from a mature seed and methods for preparing a mature embryo explant further comprise preparing slices (e.g., longitudinal slices) of the mature embryo. The mature embryo explant comprises at least one of the following tissues: leaf primordia, mesocotyl, shoot apical meristem, and root primordia. In some embodiments, the mature embryo explant comprises leaf primordia, mesocotyl, and root primordia. In some of these embodiments, the mature embryo explant further comprises a shoot apical meristem. The slices may be prepared using any method or suitable apparatus known in the art, including slices prepared by hand with a scalpel. In certain embodiments, each mature embryo is sliced into about 3 to 4 thin sections using a scalpel. The use of a dissecting microscope can aid in slicing of the mature embryo.

The mature seed from which the mature embryo or mature embryo explant is derived can be a seed of any plant. In some embodiments, the mature seed is from a monocot. In particular embodiments, the mature seed is from maize, rice, sorghum, barley, wheat, oats, or millet. In certain embodiments, the mature seed is from a recalcitrant plant, such as an elite maize inbred. As used herein, a “recalcitrant tissue” or “recalcitrant plant” is a tissue or a plant that has a low rate of transformation using traditional methods of transformation, such as those disclosed elsewhere herein. In some embodiments, the recalcitrant tissue or plant is unable to be transformed in the absence of the cell proliferation factor. In other embodiments, the recalcitrant tissue or plant has a rate of successful transformation of less than about 20%, less than about 15%, less than about 10%, less than about 5%, less than about 1%, less than about 0.1%, less than about 0.01%, less than about 0.001%, or less.

The mature embryo or mature embryo explant can be prepared from a dried mature seed. The dried mature seed can comprise about 90%, 85%, 80%, 75%, 70%, 65%, 60%, 55%, 50%, 45%, 40%, 35%, 30%, 25%, 20%, 15%, 10%, 5%, 1%, 0.1% or less water than a mature seed that has not been dried. The dried mature seed can be imbibed with an aqueous solution for a sufficient period of time to allow the dried mature seed to soften so that the mature embryo may be dissected from the seed and in some embodiments, mature embryo explant slices prepared from the mature embryo. In some embodiments, the dried mature seed is imbibed in an aqueous solution for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours or greater. In certain embodiments, the aqueous solution is water. In certain embodiments, the dried mature seed is imbibed for a sufficient period of time to induce germination of the seed. A germinated seed is one in which the radical has emerged.

Mature embryos and mature embryo explants can be transformed with a polynucleotide of interest through the provision of a cell proliferation factor (e.g., babyboom polypeptide). A heterologous polynucleotide encoding the cell proliferation factor is introduced into the mature embryo explant prior to or at the same time as the introduction of the polynucleotide of interest. The heterologous polynucleotide encoding the cell proliferation factor and the polynucleotide of interest can be provided on the same expression cassette or on separate expression cassettes.

The polynucleotides can be introduced into the mature embryo explant using any method known in the art, including but not limited to, Agrobacterium-mediated transformation.

In some embodiments, transformed mature embryo explants can be identified. Any method can be used to identify a plant cell or tissue comprising the polynucleotide of interest. In some examples, plant cells or tissues comprising the polynucleotide of interest are identified using one or more of the following techniques, including but not limited to PCR methods, hybridization methods such as Southern or Northern blots, restriction digest analyses, or DNA sequencing. In some embodiments, the transformed mature embryo explants can be identified by incubating the leaf mature embryo explants under conditions to allow for growth of a callus. In some embodiments, those mature embryo explants that are able to grow into a callus with significant proliferation indicate those mature embryo explants that have been transformed. In other embodiments, the transformed mature embryo explants can be identified and selected for through the introduction and expression of a selectable marker gene into the mature embryo explant.

Methods are also provided herein for the transformation of leaf tissues, which can be a leaf base. A leaf base is the tissue of a leaf above the first leaf base node. The leaf tissue can be derived from any plant. In some embodiments, the leaf tissue is derived from a monocot. In particular embodiments, the leaf tissue is derived from maize, rice, sorghum, barley, wheat, oats, or millet. In certain embodiments, the leaf tissue is derived from a recalcitrant plant, such as an elite maize inbred.

The leaf base can be from a mature leaf or a leaf from a seedling. As used herein, a “seedling” refers to a germinated seed or germinated embryo, or a plantlet generated in an in vitro system (e.g., from callus). The seedlings can be prepared by germinating seeds or dissecting mature embryos from mature seeds for germination. In some embodiments, the mature embryos are dissected from dried mature seeds that have been imbibed with an aqueous solution, as described herein.

In some embodiments, the coleoptile is removed from the leaf tissue and the leaf fragment is split longitudinally, and then horizontal slices are made to cross-dissect the leaf fragment into leaf tissue pieces. In particular embodiments, the pieces of leaf tissue are about 1 to 2 mm in length.

The leaf tissue can be transformed with a polynucleotide of interest through the provision of a cell proliferation factor (e.g., babyboom polypeptide). The polynucleotides can be introduced into the leaf tissue using any method known in the art, including but not limited to, Agrobacterium-mediated transformation. A heterologous polynucleotide of interest encoding the cell proliferation factor is introduced into the leaf tissue prior to or at the same time as the introduction of the polynucleotide of interest. The heterologous polynucleotide encoding the cell proliferation factor is expressed. The heterologous polynucleotide encoding the cell proliferation factor and the polynucleotide of interest can be provided on the same expression cassette or on separate expression cassettes.

In some embodiments, transformed leaf tissues can be identified. Any method can be used to identify a plant cell or tissue comprising the polynucleotide of interest. In some examples, plant cells or tissues comprising the polynucleotide of interest are identified using one or more of the following techniques, including but not limited to PCR methods, hybridization methods such as Southern or Northern blots, restriction digest analyses, or DNA sequencing. In some embodiments, the transformed leaf tissues can be identified by incubating the leaf tissues under conditions to allow for growth of a callus. In some embodiments, those leaf tissues that are able to grow a callus with significant proliferation indicate those leaf tissues that have been transformed In other embodiments, the transformed leaf tissue can be identified and selected for through the introduction and expression of a selectable marker gene into the leaf tissue.

The cells that have been transformed may be grown into plants in accordance with conventional ways. See, for example, McCormick et al. (1986) Plant Cell Rep 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, transformed seed (also referred to as “transgenic seed”) having a nucleotide construct, for example, an expression cassette, stably incorporated into their genome is provided. Thus, compositions of the invention include plant cells, plant tissues, plant parts, and plants comprising the presently disclosed polynucleotides, polypeptides, promoter constructs, expression cassettes, or vectors. Likewise, the methods of the invention can be performed in plant cells, plant tissues, plant parts, and plants.

In some embodiments, the activity and/or level of the cell proliferation factor (e.g., babyboom polypeptide, Wuschel) is reduced prior to regenerating a plant from a cell or tissue having the polynucleotide of interest. In some of these embodiments, the polynucleotide encoding the cell proliferation factor is excised prior to the regeneration of a plant. In certain embodiments, the promoter and other regulatory elements that are operably linked to the heterologous polynucleotide encoding the cell proliferation factor are excised along with the cell proliferation factor coding sequence. In certain embodiments, the polynucleotide encoding the cell proliferation factor is flanked by recombination sites and an appropriate site-specific recombinase is introduced into the mature embryo explant or callus grown therefrom to excise the polynucleotide encoding the cell proliferation factor prior to regeneration of the mature embryo explant or callus into a plant. In some of those embodiments wherein both a babyboom polypeptide and a Wuschel polypeptide are provided to the plant cell, both the polynucleotide encoding the babyboom polypeptide and the polynucleotide encoding the Wuschel polypeptide are excised. The two polynucleotides can be present on the same or different expression cassettes and, therefore, can be excised in one or two different excision reactions. In some of these embodiments, the polynucleotide encoding the site-specific recombinase for excising the babyboom and Wuschel polynucleotides can be located on the same expression cassette as the babyboom and Wuschel polynucleotides and all three polynucleotides can be excised through the activity of the site-specific recombinase.

In order to control the excision of the cell proliferation factor, the expression of the site-specific recombinase that is responsible for the excision can be controlled by a late embryo promoter or an inducible promoter. In some embodiments, the late embryo promoter is GZ (Uead et al. (1994) Mol Cell Biol 14:4350-4359), gamma-kafarin promoter (Mishra et al. (2008) Mol Biol Rep 35:81-88), Glb1 promoter (Liu et al. (1998) Plant Cell Reports 17:650-655), ZM-LEG1 (U.S. Pat. No. 7,211,712), EEP1 (U.S. Patent Application No. US 2007/0169226), B22E (Klemsdal et al. (1991) Mol Gen Genet 228:9-16), or EAP1 (U.S. Pat. No. 7,321,031). In some embodiments, the inducible promoter that regulates the expression of the site-specific recombinase is a heat-shock, light-induced promoter, a drought-inducible promoter, including but not limited to Hval (Straub et al. (1994) Plant Mol Biol 26:617-630), Dhn, and WSI18 (Xiao & Xue (2001) Plant Cell Rep 20:667-673). In other embodiments, expression of the site-specific recombinase is regulated by the maize rab17 promoter, or one of the presently disclosed promoter constructs (e.g., maize rab17 promoter and an attB site). In some embodiments, the site-specific recombinase that excises the polynucleotide encoding the cell proliferation factor is FLP or Cre.

Any plant species can be transformed, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena), barley (Hordeum), Arabidopsis, switchgrass, vegetables, ornamentals, grasses, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean and sugarcane plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

As used herein, the term plant also includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

If the polynucleotide of interest is introduced into an organism, it may impart various changes in the organism, particularly plants, including, but not limited to, modification of the fatty acid composition in the plant, altering the amino acid content of the plant, altering pathogen resistance, and the like. These results can be achieved by providing expression of heterologous products, increased expression of endogenous products in plants, or suppressed expression of endogenous produces in plants.

General categories of polynucleotides of interest include, for example, those genes involved in information, such as zinc fingers, those involved in communication, such as kinases, those involved in biosynthetic pathways, and those involved in housekeeping, such as heat shock proteins. More specific categories of transgenes, for example, include sequences encoding important traits for agronomics, insect resistance, disease resistance, herbicide resistance, sterility, grain characteristics, oil, starch, carbohydrate, phytate, protein, nutrient, metabolism, digestibility, kernel size, sucrose loading, and commercial products.

Traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Protein modifications to alter amino acid levels are described in U.S. Pat. Nos. 5,703,049, 5,885,801, 5,885,802, and 5,990,389 and WO 98/20122, herein incorporated by reference.

Insect resistance genes may encode resistance to pests such as rootworm, cutworm, European Corn Borer, and the like. Such genes include, for example, Bacillus thuringiensis toxic protein genes (U.S. Pat. Nos. 5,366,892; 5,747,450; 5,737,514; 5,723,756; 5,593,881; and Geiser et al. (1986) Gene 48:109); lectins (Van Damme et al. (1994) Plant Mol. Biol. 24:825); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the S4 and/or Hra mutations in ALS), genes coding for resistance to herbicides that act to inhibit action of glutamine synthase, such as phosphinothricin or basta (e.g., the bar gene), genes providing resistance to glyphosate, such as GAT (glyphosate N-acetyltransferase; U.S. Pat. No. 6,395,485), EPSPS (enolpyruvylshikimate-3-phosphate synthase; U.S. Pat. Nos. 6,867,293, 5,188,642, 5,627,061), or GOX (glyphosate oxidoreductase; U.S. Pat. No. 5,463,175), or other such genes known in the art. The nptII gene encodes resistance to the antibiotics kanamycin and geneticin.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

Commercial traits can also be encoded on a gene or genes that could, for example increase starch for ethanol production, or provide expression of proteins.

Reduction of the activity of specific genes (also known as gene silencing, or gene suppression) is desirable for several aspects of genetic engineering in plants. Many techniques for gene silencing are well known to one of skill in the art, including but not limited to antisense technology (see, e.g., Sheehy et al. (1988) Proc. Natl. Acad. Sci. USA 85:8805-8809; and U.S. Pat. Nos. 5,107,065; 5,453,566; and 5,759,829); cosuppression (e.g., Taylor (1997) Plant Cell 9:1245; Jorgensen (1990) Trends Biotech. 8(12):340-344; Flavell (1994) Proc. Natl. Acad. Sci. USA 91:3490-3496; Finnegan et al. (1994) Bio Technology 12: 883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; Javier (2003) Nature 425:257-263; and, Montgomery et al. (1998) Proc. Natl. Acad. Sci. USA 95:15502-15507), virus-induced gene silencing (Burton, et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; WO 02/00904; and WO 98/53083); ribozymes (Steinecke et al. (1992) EMBO J. 11:1525; U.S. Pat. No. 4,987,071; and, Perriman et al. (1993) Antisense Res. Dev. 3:253); oligonucleotide mediated targeted modification (e.g., WO 03/076574 and WO 99/25853); Zn-finger targeted molecules (e.g., WO 01/52620; WO 03/048345; and WO 00/42219); and other methods or combinations of the above methods known to those of skill in the art.

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, California, USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the babyboom polynucleotide. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, New York).

For example, the entire babyboom polynucleotide, or one or more portions thereof, may be used as a probe capable of specifically hybridizing to corresponding babyboom polynucleotide and messenger RNAs. To achieve specific hybridization under a variety of conditions, such probes include sequences that are unique among babyboom polynucleotide sequences and are optimally at least about 10 nucleotides in length, and most optimally at least about 20 nucleotides in length. Such probes may be used to amplify corresponding babyboom polynucleotide from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, New York).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1× SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, New York).

It is to be noted that the term “a” or “an” entity refers to one or more of that entity; for example, “a polypeptide” is understood to represent one or more polypeptides. As such, the terms “a” (or “an”), “one or more,” and “at least one” can be used interchangeably herein.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the presently disclosed subject matter be limited to the specific values recited when defining a range.

The following examples are offered by way of illustration and not by way of limitation.

### EXPERIMENTAL

### Example 1. a Modified Rab17 Promoter for the Regulated Expression of Genes

Gateway™ technology (Invitrogen, Carlsbad, CA) was used to place Gateway™ recombination sites between a promoter and a coding sequence, and between the coding sequence and a terminator. The product of a Gateway™ reaction set up in this manner leaves attB sites in those locations.

The rab17 promoter was identified as a candidate for regulating the expression of FLP recombinase for excision of polynucleotides encoding cell proliferation factors in tissue culture. It was tested for FLP/FRT excision of cell proliferation factor genes in culture. The PHP31004 plasmid was constructed, which has the following operably linked components: Rab17 Pro-attB1::FLPm-attB2::PinII+Ubi Pro-FRT1::CFP::PinII+Ubi Pro::ZmBBM::PinII-FRT1::YFP::PinII+Ubi Pro::moPAT::PinII. The sequence of the expression cassette for the FLPm gene in the PHP31004 plasmid is provided in SEQ ID NO: 46.

After excision by the FLP recombinase, the PHP31004 plasmid has the following operably linked components: Rab17 Pro-attB1::FLPm-attB2::PinII+Ubi Pro-FRT1::YFP::PinII+Ubi Pro::moPAT::PinII.

A plasmid (PHP30642) lacking the attB sites, but comprising the FLPm gene was constructed. The PHP30642 has the following operably linked components: Rab17 pro::FLPm::Gz-W64A term+Ubi pro-FRT1::CFP::PinII+UbiPro::ZmBBM::PinII-FRT1::YFP::PinII+Ubi Pro::moPAT::PinII. The sequence of the expression cassette for the FLPm gene in the PHP30642 plasmid is provided in SEQ ID NO: 47.

After excision by the FLP recombinase, the PHP30642 plasmid has the following operably linked components: Rab17 pro::FLPm::Gz-W64A term+Ubi pro-FRT1::YFP::PinII+Ubi pro::moPAT::PinII. The construct lacking the attB sites resulted in frequent premature excision of the cell proliferation factor genes.

### Example 2. Transformation of Maize Immature Embryos

Transformation can be accomplished by various methods known to be effective in plants, including particle-mediated delivery, Agrobacterium-mediated transformation, PEG-mediated delivery, and electroporation.

a. Particle-Mediated Delivery

Transformation of maize immature embryos using particle delivery is performed as follows. Media recipes follow below.

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

A plasmid comprising the Zm-BBM (also referred to as Zm-ODP2) coding sequence (set forth in SEQ ID NO: 9) operably linked to a promoter is constructed. This could be a weak promoter such as nos, a tissue-specific promoter, such as globulin-1 or oleosin, an inducible promoter such as In2, or a strong promoter such as ubiquitin plus a plasmid containing the selectable marker gene phosphinothricin N-acetyltransferase (PAT; Wohlleben et al. (1988) Gene 70:25-37) that confers resistance to the herbicide bialaphos. The plasmid DNA containing the selectable marker gene PAT and the BBM plasmid are precipitated onto 1.1 μm (average diameter) tungsten pellets using a calcium chloride (CaCl₂)) precipitation procedure by mixing 100 μl prepared tungsten particles in water, 10 μl (1 μg) DNA in Tris EDTA buffer (1 μg total DNA), 100 μl 2.5 M CaCl₂, and 10 μl 0.1 M spermidine. Each reagent is added sequentially to the tungsten particle suspension, with mixing. The final mixture is sonicated briefly and allowed to incubate under constant vortexing for 10 minutes. After the precipitation period, the tubes are centrifuged briefly, liquid is removed, and the particles are washed with 500 ml 100% ethanol, followed by a 30 second centrifugation. Again, the liquid is removed, and 105 μl 100% ethanol is added to the final tungsten particle pellet. For particle gun bombardment, the tungsten/DNA particles are briefly sonicated. 10 μl of the tungsten/DNA particles is spotted onto the center of each macrocarrier, after which the spotted particles are allowed to dry about 2 minutes before bombardment.

The sample plates are bombarded at level #4 with a Biorad Helium Gun. All samples receive a single shot at 450 PSI, with a total of ten aliquots taken from each tube of prepared particles/DNA.

Following bombardment, the embryos are incubated on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to a lighted culture room. Approximately 7-10 days later, developing plantlets are transferred to 272V hormone-free medium in tubes for 7-10 days until plantlets are well established. Plants are then transferred to inserts in flats (equivalent to a 2.5″ pot) containing potting soil and grown for 1 week in a growth chamber, subsequently grown an additional 1-2 weeks in the greenhouse, then transferred to Classic 600 pots (1.6 gallon) and grown to maturity. Plants are monitored and scored for transformation efficiency, and/or modification of regenerative capabilities.

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature).

Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.).

Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

b. Agrobacterium-Mediated Transformation

Agrobacterium-mediated transformation was performed essentially as described in Djukanovic et al. (2006) Plant Biotech J 4:345-57. Briefly, 10-12 day old immature embryos (0.8-2.5 mm in size) were dissected from sterilized kernels and placed into liquid medium (4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.690 g/L L-proline, 68.5 g/L sucrose, 36.0 g/L glucose, pH 5.2). After embryo collection, the medium was replaced with 1 ml Agrobacterium at a concentration of 0.35-0.45 OD₅₅₀. Maize embryos were incubated with Agrobacterium for 5 min at room temperature, then the mixture was poured onto a media plate containing 4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.690 g/L L-proline, 30.0 g/L sucrose, 0.85 mg/L silver nitrate, 0.1 nM acetosyringone, and 3.0 g/L Gelrite, pH 5.8. Embryos were incubated axis down, in the dark for 3 days at 20° C., then incubated 4 days in the dark at 28° C., then transferred onto new media plates containing 4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.69 g/L L-proline, 30.0 g/L sucrose, 0.5 g/L MES buffer, 0.85 mg/L silver nitrate, 3.0 mg/L Bialaphos, 100 mg/L carbenicillin, and 6.0 g/L agar, pH 5.8. Embryos were subcultured every three weeks until transgenic events were identified. Somatic embryogenesis was induced by transferring a small amount of tissue onto regeneration medium (4.3 g/L MS salts (Gibco 11117), 5.0 ml/L MS Vitamins Stock Solution, 100 mg/L myo-inositol, 0.1 M ABA, 1 mg/L IAA, 0.5 mg/L zeatin, 60.0 g/L sucrose, 1.5 mg/L Bialaphos, 100 mg/L carbenicillin, 3.0 g/L Gelrite, pH 5.6) and incubation in the dark for two weeks at 28° C. All material with visible shoots and roots were transferred onto media containing 4.3 g/L MS salts (Gibco 11117), 5.0 ml/L MS Vitamins Stock Solution, 100 mg/L myo-inositol, 40.0 g/L sucrose, 1.5 g/L Gelrite, pH 5.6, and incubated under artificial light at 28° C. One week later, plantlets were moved into glass tubes containing the same medium and grown until they were sampled and/or transplanted into soil.

### Example 3. Transient Expression of BBM Enhances Transformation

Parameters of the transformation protocol can be modified to ensure that the BBM activity is transient. One such method involves precipitating the BBM-containing plasmid in a manner that allows for transcription and expression, but precludes subsequent release of the DNA, for example, by using the chemical PEI.

In one example, the BBM plasmid is precipitated onto gold particles with PEI, while the transgenic expression cassette (UBI::moPAT˜GFPm::PinII; moPAT is the maize optimized PAT gene) to be integrated is precipitated onto gold particles using the standard calcium chloride method.

Briefly, gold particles were coated with PEI as follows. First, the gold particles were washed. Thirty-five mg of gold particles, 1.0 in average diameter (A.S.I. #162-0010), were weighed out in a microcentrifuge tube, and 1.2 ml absolute EtOH was added and vortexed for one minute. The tube was incubated for 15 minutes at room temperature and then centrifuged at high speed using a microfuge for 15 minutes at 4° C. The supernatant was discarded and a fresh 1.2 ml aliquot of ethanol (EtOH) was added, vortexed for one minute, centrifuged for one minute, and the supernatant again discarded (this is repeated twice). A fresh 1.2 ml aliquot of EtOH was added, and this suspension (gold particles in EtOH) was stored at −20° C. for weeks. To coat particles with polyethylimine (PEI; Sigma #P3143), 250 μl of the washed gold particle/EtOH mix was centrifuged and the EtOH discarded. The particles were washed once in 100 μl ddH₂O to remove residual ethanol, 250 μl of 0.25 mM PEI was added, followed by a pulse-sonication to suspend the particles and then the tube was plunged into a dry ice/EtOH bath to flash-freeze the suspension, which was then lyophilized overnight. At this point, dry, coated particles could be stored at −80° C. for at least 3 weeks. Before use, the particles were rinsed 3 times with 250 μl aliquots of 2.5 mM HEPES buffer, pH 7.1, with 1× pulse-sonication, and then a quick vortex before each centrifugation. The particles were then suspended in a final volume of 250 μl HEPES buffer. A 25 μl aliquot of the particles was added to fresh tubes before attaching DNA. To attach uncoated DNA, the particles were pulse-sonicated, then 1 g of DNA (in 5 μl water) was added, followed by mixing by pipetting up and down a few times with a Pipetteman and incubated for 10 minutes. The particles were spun briefly (i.e. 10 seconds), the supernatant removed, and 60 μl EtOH added. The particles with PEI-precipitated DNA-1 were washed twice in 60 μl of EtOH. The particles were centrifuged, the supernatant discarded, and the particles were resuspended in 45 μl water. To attach the second DNA (DNA-2), precipitation using TFX-50 was used. The 45 μl of particles/DNA-1 suspension was briefly sonicated, and then 5 μl of 100 ng/μl of DNA-2 and 2.5 μl of TFX-50 were added. The solution was placed on a rotary shaker for 10 minutes, centrifuged at 10,000 g for 1 minute. The supernatant was removed, and the particles resuspended in 60 μl of EtOH. The solution was spotted onto macrocarriers and the gold particles onto which DNA-1 and DNA-2 had been sequentially attached were delivered into scutellar cells of 10 DAP Hi-II immature embryos using a standard protocol for the PDS-1000. For this experiment, the DNA-1 plasmid contained a UBI::RFP::pinII expression cassette, and DNA-2 contained a UBI::CFP::pinII expression cassette. Two days after bombardment, transient expression of both the CFP and RFP fluorescent markers was observed as numerous red & blue cells on the surface of the immature embryo. The embryos were then placed on non-selective culture medium and allowed to grow for 3 weeks before scoring for stable colonies. After this 3-week period, 10 multicellular, stably-expressing blue colonies were observed, in comparison to only one red colony. This demonstrated that PEI-precipitation could be used to effectively introduce DNA for transient expression while dramatically reducing integration of the PEI-introduced DNA and thus reducing the recovery of RFP-expressing transgenic events. In this manner, PEI-precipitation can be used to deliver transient expression of BBM and/or WUS2.

For example, the particles are first coated with UBI::BBM::pinII using PEI, then coated with UBI::moPAT˜YFP using TFX-50, and then bombarded into scutellar cells on the surface of immature embryos. PEI-mediated precipitation results in a high frequency of transiently expressing cells on the surface of the immature embryo and extremely low frequencies of recovery of stable transformants (relative to the TFX-50 method). Thus, it is expected that the PEI-precipitated BBM cassette expresses transiently and stimulates a burst of embryogenic growth on the bombarded surface of the tissue (i.e. the scutellar surface), but this plasmid will not integrate. The PAT˜GFP plasmid released from the Ca⁺⁺/gold particles is expected to integrate and express the selectable marker at a frequency that results in substantially improved recovery of transgenic events. As a control treatment, PEI-precipitated particles containing a UBI::GUS::pinII (instead of BBM) are mixed with the PAT˜GFP/Ca⁺⁺ particles. Immature embryos from both treatments are moved onto culture medium containing 3 mg/l bialaphos. After 6-8 weeks, it is expected that GFP+, bialaphos-resistant calli will be observed in the PEI/BBM treatment at a much higher frequency relative to the control treatment (PEI/GUS).

As an alternative method, the BBM plasmid is precipitated onto gold particles with PEI, and then introduced into scutellar cells on the surface of immature embryos, and subsequent transient expression of the BBM gene elicits a rapid proliferation of embryogenic growth. During this period of induced growth, the explants are treated with Agrobacterium using standard methods for maize (see Example 1), with T-DNA delivery into the cell introducing a transgenic expression cassette such as UBI::moPAT˜GFPm::pinII. After co-cultivation, explants are allowed to recover on normal culture medium, and then are moved onto culture medium containing 3 mg/l bialaphos. After 6-8 weeks, it is expected that GFP⁺, bialaphos-resistant calli will be observed in the PEI/BBM treatment at a much higher frequency relative to the control treatment (PEI/GUS).

It may be desirable to “kick start” callus growth by transiently expressing the BBM and/or WUS2 polynucleotide products. This can be done by delivering BBM and WUS2 5′-capped polyadenylated RNA, expression cassettes containing BBM and WUS2 DNA, or BBM and/or WUS2 proteins. All of these molecules can be delivered using a biolistics particle gun. For example 5′-capped polyadenylated BBM and/or WUS2 RNA can easily be made in vitro using Ambion's mMessage mMachine kit. RNA is co-delivered along with DNA containing a polynucleotide of interest and a marker used for selection/screening such as Ubi::moPAT˜GFPm::PinII. It is expected that the cells receiving the RNA will immediately begin dividing more rapidly and a large portion of these will have integrated the agronomic gene. These events can further be validated as being transgenic clonal colonies because they will also express the PAT˜GFP fusion protein (and thus will display green fluorescence under appropriate illumination). Plants regenerated from these embryos can then be screened for the presence of the polynucleotide of interest.

### Example 4. Excision of Genes Encoding Cell Proliferation Factors

a. Rab17::CRE

The following T-DNA was constructed: RB-Ubi pro-loxP::Rab17 pro-attB1::Cre-attB2::PinII+NOS::ZmWUS2::PinII+Ubi pro::ZmBBM::PinII-loxP::YFP::PinII+Ubi pro::moPAT::PinII-LB. As a control, a T-DNA containing Ubi pro::moPAT::PinII was constructed. These T-DNA are introduced into immature embryos (approximately 0.8-2.5 mm in length) of the maize inbred PHH5G using standard Agrobacterium-mediated transformation methods. Non-transformed immature embryos of this inbred swell and initiate a small volume of callus cells, but proliferation does not occur on media compositions typically used for maize tissue culture (for example, 605J media, which comprises 4.3 g/l MS salts, 0.6 g/l Shenk & Hildebrand vitamins, 100 mg/l calcium chloride, 275 mg/l ammonium sulfate, 275 mg/l ammonium sulfate, 240 mg/l potassium phosphate, 100 mg/l magnesium sulfate, 3.4 g/l potassium nitrate, 1.8 mg/l boric acid, 6 mg/l manganese sulfate, 0.15 mg/l sodium molybdate, 0.5 mg/l potassium iodide, 22 mg/l disodium EDTA, 17 mg/l ferrous sulfate, 3.4 mg/l silver nitrate, 1 g/l L-proline, 0.2 mg/l nicotinic acid, 0.4 mg/l thiamine, 0.2 mg/l pyridoxine, 0.8 mg/l glycine, 100 mg/l carbenicillin, 0.8 mg/l 2-4D, 1.2 mg/l dicamba, 0.3 g/l casein hydrosylate, 20 g/l sucrose, 0.6 g/l glucose, and 6 g/l TC agar, pH 5.8). Likewise, PHH5G immature embryos transformed with Ubi pro::moPAT::PinII alone do not produce healthy, growing callus, irrespective of whether bialaphos selection is provided. Thus, no transformed events were produced after introducing Ubi pro::moPAT::PinII alone (or with Ubi pro::moPAT::PinII+Ubi pro::YFP::PinII). In contrast, when the genes encoding cell proliferation factors (BBM and WUS2)+Ubi pro::moPAT::PinII were introduced into PHH5G immature embryos, vigorously-growing callus transformants were recovered from 45% of the treated embryos. To remove the genes encoding cell proliferation factors, the Rab17 promoter can be induced through exposure to either 20 mM abscisic acid (ABA), 20-30% sucrose, or desiccation. In this experiment, callus was placed on dry filter papers for three days to induce excision, and then transferred to regeneration medium. If callus was not treated to induce the expression of Cre recombinase, excision of the genes encoding cell proliferation factors did not occur and viable plantlets were not regenerated. However, for events that were taken through the desiccation treatment, Cre excision occurred in over 90% of single copy events (activating YFP) and subsequent regeneration was not inhibited. Transgenic plants were screened using combinations of PCR primers designed to detect the presence of the Ubi pro-loxP::YFP junction formed as a result of excision, and moPAT (not effected by excision), and the absence of Cre, WUS2 and BBM. Plants in which excision was complete were grown to maturity and were either selfed or out-crossed to wild-type plants. Transgenic progeny seed were readily identified through the yellow fluorescence phenotype and plants were easily tracked through either BASTA resistance or yellow fluorescence. PCR analysis in both the T1 and T2 generations indicated that only the excised locus was present in a single genomic copy and that no Agrobacterium plasmid backbone was present.

Both FLP and Cre recombinase have been successfully used to excise genes encoding cell proliferation factors before regeneration. The following two constructs represent examples of how the recombinases can be used for controlled excision:

$$PHP 32371 - FLP / FRT$$

$$RB - Ubi - FRT 1 \text{::} CFP \text{::} PinII - attB 4 + Rab 17 Pro - attB 1 \text{::} FLP - attB 2 \text{::} PinII + Nos \text{::} ZmWUS 2 \text{::} PinII + Ubi \text{::} ZmBBM \text{::} PinII - FRT 1 \text{::} YFP \text{::} PinII + Ubi \text{::} moPAT \text{::} PinII - LB$$

$$The T - DNA sequence of PHP 32371 is set forth in SEQ ID NO : 110.$$

$$PHP 35648 - Cre / LoxP$$

$$RB - Ubi - LoxP \text{::} CFP \text{::} PinII - attB 4 + Rab 17 Pro - attb 1 \text{::} Cre - attB 2 \text{::} PinII + Nos \text{::} ZmWUS 2 \text{::} PinII + Ubi \text{::} ZmBBM \text{::} PinII - LoxP \text{::} YFP \text{::} PinII + Ubi \text{::} MOPAT \text{::} PinII - LB$$

The T-DNA sequence of PHP35648 is set forth in SEQ ID NO: 111.

For both recombinases, expression was controlled by the Rab17 promoter (Vilardell et al. (1991) Plant Mol. Biol 17:985-993) with the attb1 site.

For both constructs, transgenic callus events were readily recovered, and both constructs worked well for excision of the expression cassettes comprising genes encoding cell proliferation factors (see Table 2). Of the total number of calli exposed to the 3-day desiccation treatment, 61% (Cre) and 29% (FLP) of the resultant plants exhibited a normal wild-type phenotype. As confirmation of excision, PCR analysis in both the T1 and T2 generations indicated that only the excised locus was present in a single genomic copy and that no Agrobacterium plasmid backbone was present.

TABLE 2
Desiccation-induced excision of the recombinase, BBM
& WUS expression cassettes prior to regeneration.

| | # of Callus events | # of event swith | | | # with a totally- |
|---|---|---|---|---|---|
| | exposed to | normal T0 plant | # of T0 plantlets | # of single | excised DevGene |
| | desiccation | phenotype | analyzed using PCR | copy plants | package |
| PHP35648 | 180 | 110 (61%) | 168 | 94 (56%) | 81 (86%) |
| PHP32371 | 118 | 34 (29%) | 75 | 51 (68%) | 31 (61%) |

Additional constructs that utilize Cre/LoxP were generated.

$$PHP 46446 : RB - LoxP - Rab 17 Pro - attB 1 \text{::} Cre - attB 2 \text{::} PinII + Nos \text{::} Zm - WUS 2 \text{::} PinII \text{::} GZ - W 64 A Term - attB 2 + Ubi \text{::} ZmBBM \text{::} PinII - LoxP - LB$$

$$PHP 48733 : RB - LoxP - Rab 17 Pro - attB 1 \text{::} Cre - attB 2 \text{::} PinII + Nos : ZmWUS 2 \text{::} PinII + Ubi \text{::} ZmBBM \text{::} PinII - LoxP - LB$$

The T-DNA sequences of PHP46446 and PHP48733 is set forth in SEQ ID NO: 112 and 113, respectively.

Introduction of PHP35648, PHP48733, or PHP46446 into PHH5G immature maize embryos via Agrobacterium resulted in a transformation frequency of 46%, 67%, or 37%, respectively (see Table 3).

TABLE 3
Transformation of PHH5G immature maize embryos with
maize BBM and WUS2 cell proliferation factors.

| | | | | Transformation |
|---|---|---|---|---|
| | No. of | No. of | No. of callus | frequency at |
| Construct | ears | embryos | events | callus level |
| PHP35648 | 14 | 589 | 268 | 45.5 |
| PHP48733 | 14 | 584 | 389 | 66.6 |
| PHP46446 | 14 | 547 | 203 | 37.1 |

The use of the PHP35648, PHP48733, PHP46446, and PHP32371 constructs (all of which comprised the Rab17 promoter (Vilardell et al. (1991) Plant Mol. Biol 17:985-993) with the attb1 site regulating the expression of the recombinase), did not result in frequent premature excision of the cell proliferation factor genes, similar to the results presented in Example 1 with the PHP31004 construct.

b. Tetracycline-Inducible CRE

A 35S promoter in which three tetracycline operator sequences (Top3) have been introduced in proximity to the TATA box (Gatz et al. (1992) Plant J 2:397-404) was operably linked to the CRE structural gene in the following T-DNA which also includes an expression cassette for the tetracycline repressor (TETR), BBM, WUS2, and moPAT, as follows:

$$RB - loxP - 35 S \text{::} Top 3 \text{::} CRE \text{::} PinII + Ubi pro \text{::} TETR \text{::} PinII + NOS \text{::} ZmWUS 2 \text{::} PII + UBI \text{::} ZmBBM \text{::} PinII - loxP + UBI \text{::} moPAT \text{::} PinII - LB$$

After Agrobacterium-mediated transformation of 12 DAP PH581 immature embryos, followed by 6 weeks of selection on 3 mg/l bialaphos, embryos into which the control T-DNA was introduced (RB-UBI::moPAT::PinII-LB) produced transformed events at a 1% frequency. In contrast, when the above T-DNA containing ZmBBM & ZmWUS2 was transformed into immature embryos harvested from the same PH581 ears, transgenic calli were recovered at a 15% frequency. Before regenerating plantlets, callus is moved onto medium containing 0.5 mg/l tetracycline for 1 week to induce CRE-mediated excision of CRE, WUS and BBM expression cassettes. Glufosinate ammonium-resistant plants are then readily regenerated.

### Example 5. Control of BBM and WUS Expression with Regulated Promoters to Increase Transformation Frequencies

### A. OLE PRO::BBM

In the inbred PH581 maize line, the introduction of UBI::ZmBBM+NOS:ZmWUS2 increased transformation frequencies from <1% in the control treatment (UBI PRO::moPAT::PinII alone) to 15%. However, such strong over-expression of BBM negatively affects the regeneration of plantlets. Therefore, an oleosin promoter having high levels of expression in callus, with little to no activity during vegetative growth was used to express BBM. When OLE::ZmBBM::PinII+NOS::ZmWUS2::PinII was introduced into PH581 on a first T-DNA and UBI PRO::moPAT::PinII was introduced into the same cells on a second T-DNA, callus transformants were recovered at a 25% frequency. Normal, fertile plants were regenerated and crossed to wild-type PH581. T1 progeny in which the cell proliferation gene locus had segregated away from the UBI PRO::moPAT::PinII locus were readily recovered.

b. Tetracycline-Inducible BBM and WUS2

A 35S promoter in which three tetracycline operator sequences have been introduced in proximity to the TATA box (Gatz et al. (1992) Plant J 2:397-404) is operably linked to both the BBM and WUS2 genes, and these expression cassettes are put into a T-DNA along with an expression cassette for the tetracycline repressor (TETR) as follows.

$$RB - 35 S - Top 3 \text{::} ZBBM \text{::} PinII + 35 S - Top 3 \text{::} ZmWUS 2 \text{::} PinII + UBI \text{::} moPAT \text{::} PinII - LB$$

Following Agrobacterium-mediated transformation of Hi-II immature maize embryos, the embryos are transferred to selection medium 560R with 3 mg/l bialaphos+/−0.5 mg/l tetracycline. In the control treatment in which only the UBI::moPAT::PinII expression cassette is introduced, the transformation frequency is typically around 5-10%. For embryos in which the inducible BBM and WUS2 genes are introduced, transformation frequency is expected to be greatly increased upon the addition of tetracycline to the medium.

### Example 6. Regulated Expression of BBM and WUS2 for Re-Transformation

Stable transgenic events in PHH5G are produced that express ZmBBM and ZmWUS2 in a regulated fashion, for example, having BBM and WUS2 under the control of the OLE and NOS promoters, respectively, or having them being driven by a tetracycline-inducible promoter. Immature embryos are then harvested and re-transformed using Agrobacterium to deliver UBI::moPAT::PinII. PHH5G embryos not expressing BBM and WUS2 (i.e. wild-type control embryos) produce no transformation events. However, embryos expressing OLE PRO::ZmBBM::PinII and NOS PRO::ZmWUS2::PinII are expected to produce a much higher frequency of bialaphos-resistant events. Regulated expression of the genes encoding cell proliferation factors is expected to enhance the regeneration frequency of normal fertile plants, and the cell proliferation gene locus should readily segregate away from the newly-generated “trait” locus (represented here by the UBI::moPAT::PinII locus). Likewise, when the expression of tetracycline-inducible genes encoding cell proliferation factors are stimulated by the addition of 0.5 mg/l tetracycline, Agrobacterium-mediated transformation to deliver the RB-UBI::moPAT::PinII-LB T-DNA is expected to result in enhanced transformation frequencies.

### Example 7. Two T-DNA Co-Transformation to Deliver Genes Encoding Cell Proliferation Factors and Trait Genes Separately

An Agrobacterium was modified to contain two engineered plasmids, each containing a separate T-DNA. T-DNA-1 was PHP35648 (see Example 4 for description), and T-DNA-2 (PHP41877) contained RB-attB4-UBI::moPAT::PinII+UBI-FRT1::RFP::PinII-attB1+UBI::GAT::PinII-attB2-FRT87-attB3-LB (GAT=glyphosate-N-acetyltransferase) representing the T-DNA that will contain the desired stack of trait genes). Agrobacterium-mediated transformation of PHHSG immature maize embryos was followed by glyphosate selection. Only embryos that have integrated T-DNA-1 grew since growth in culture for PHH5G only occurred when the ZmWUS2 and ZmBBM genes were present. Only embryos containing T-DNA-2 were glyphosate-resistant and exhibited red fluorescence. Thus, only embryos that were co-transformed with both T-DNAs grew on glyphosate.

### Example 8. Identification of BBM Motifs

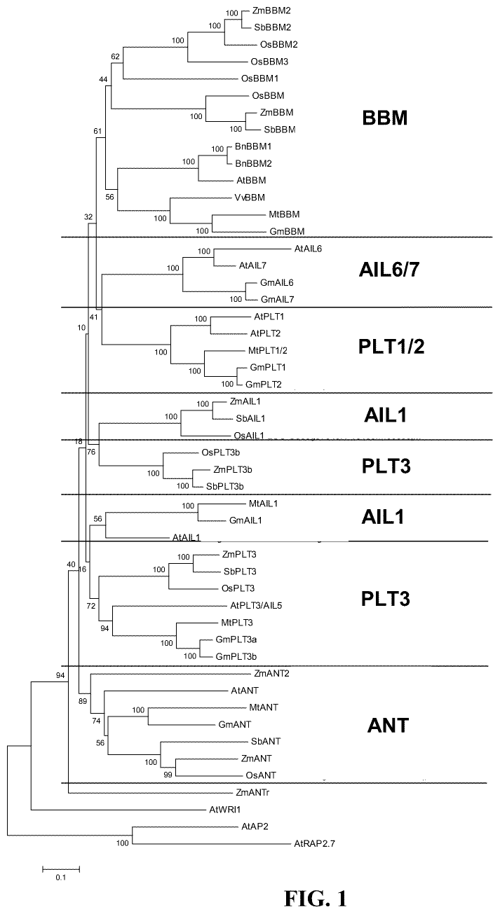

Fifty genes from different plant species were identified through a homology search using the maize BBM amino acid sequence (SEQ ID NO: 10) queried against annotated protein sequences (see FIG. 1). The gene structure and sequences of these BBM homologs were manually inspected and compared with EST/cDNA alignments whenever possible. The fifty polypeptides are set forth in SEQ ID NOs: 2, 4, 6, 8, 10, 12, 14, 16, 18, 20, 22, 24, 26, 28, 67, and 70-104. To systematically identify possible motifs within the BBM homologs, protein sequences of these fifty homologs were submitted to the MEME web server, available on the world wide web at meme.nbcr.net/meme4_1/cgi-bin/meme.cgi, with the following specific parameters:
Number of different motifs: 20
Minimum motif width: 5
Maximum motif width: 300
Minimum number of sites: 5

Default values were applied for all other parameters. The raw results from MEME were manually compared with multiple sequence alignments generated by clustalw. Only those candidates showing good consensus with the sequence alignments were considered as motifs for further analysis.

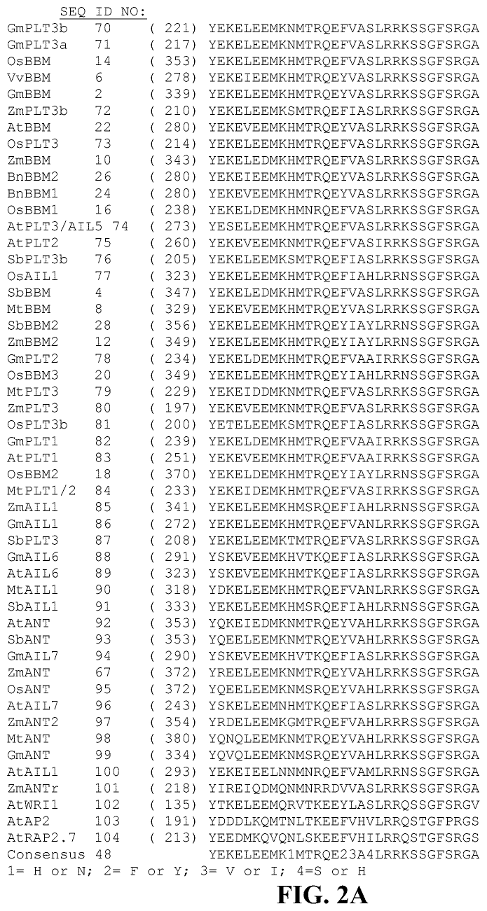

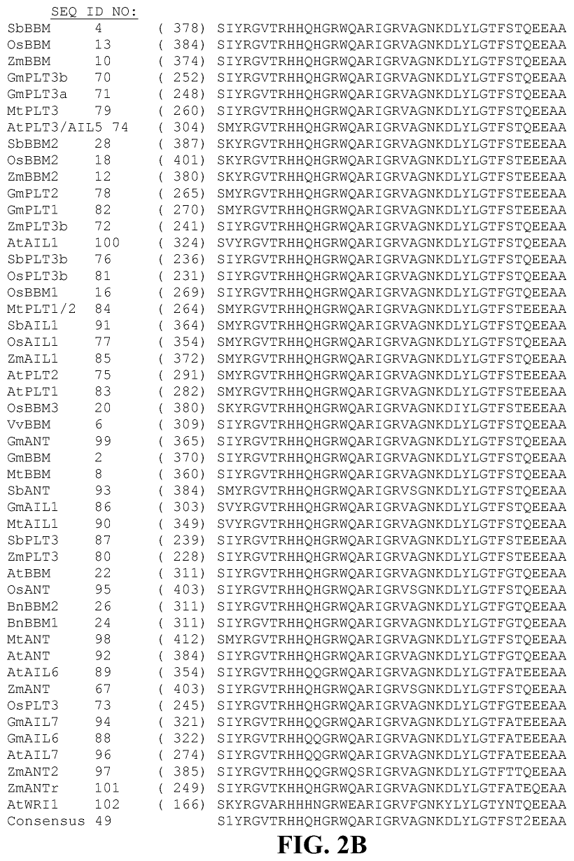

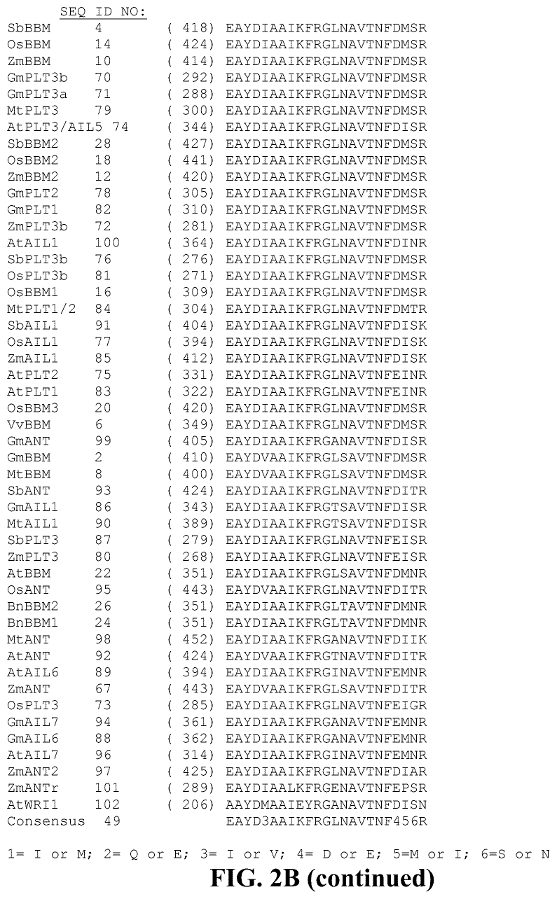

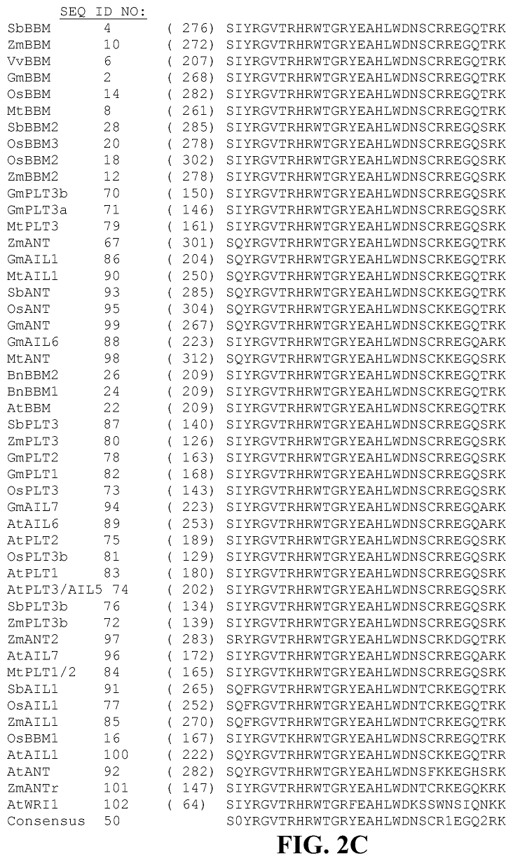

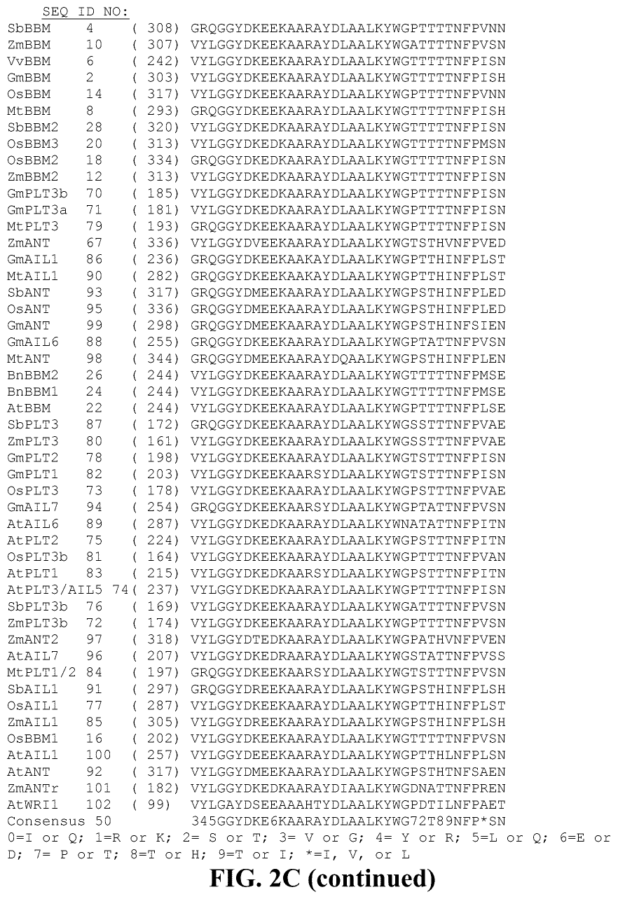

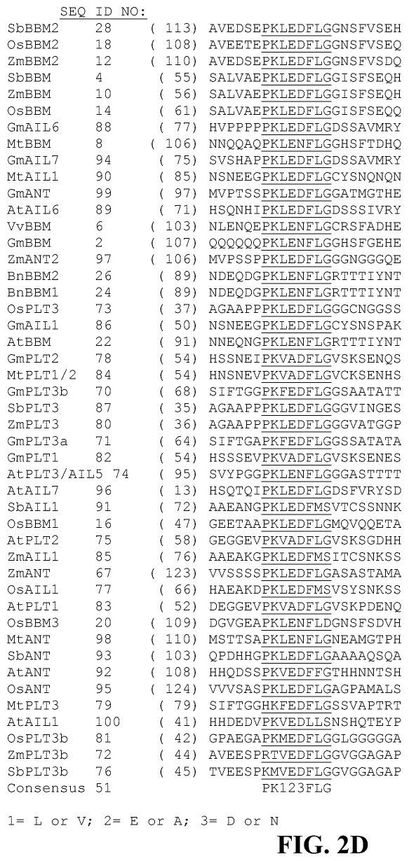

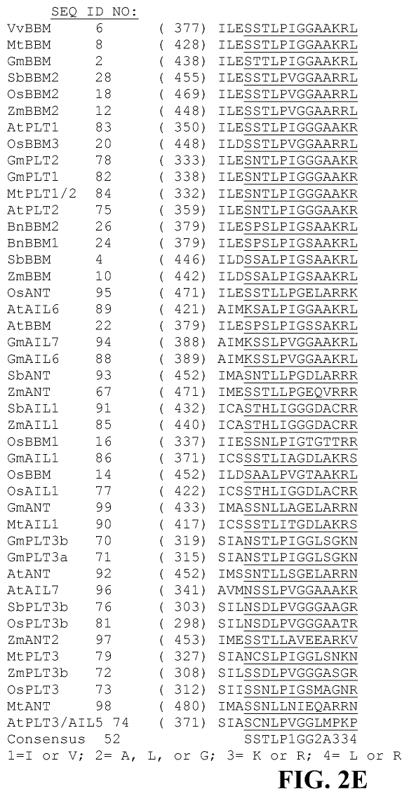

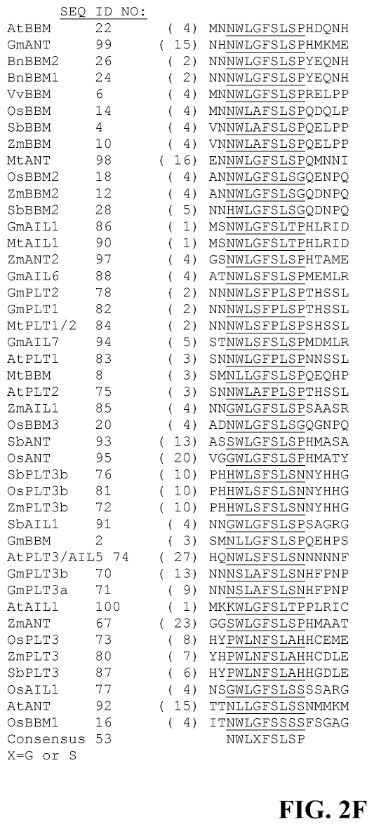

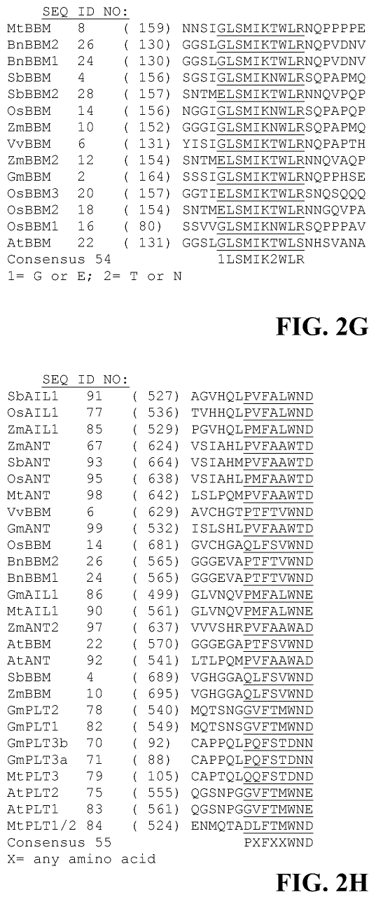

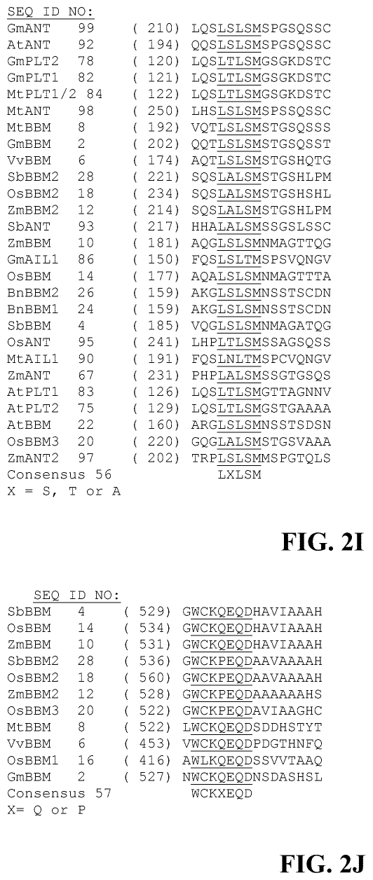

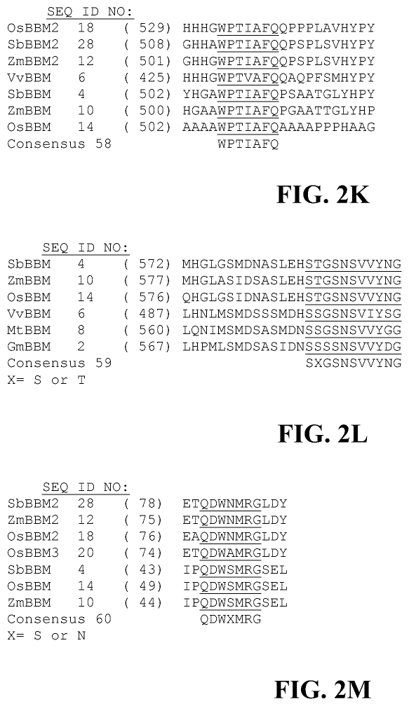

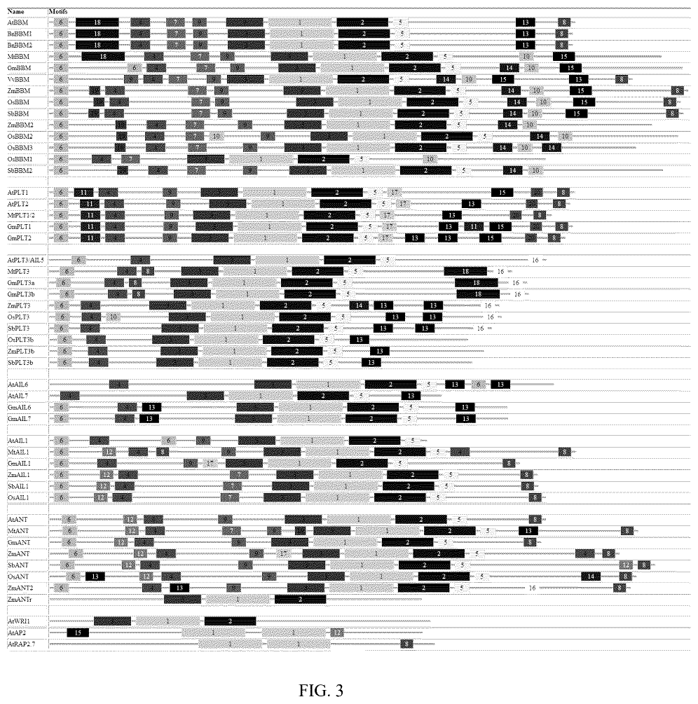

The fifty genes were subjected to a phylogenetic analysis and a total of six subgroups were identified, including BBM, PLT3, PLT1/2, AIL6/7, AIL1, and ANT (see FIG. 1). FIG. 3 depicts all 50 sequences with each of the motifs that were identified using the MEME web server. FIG. 2 provides the motif consensus sequences along with alignments of the various polypeptides used by the MEME web server to generate the consensus motif. With a few exceptions, motifs 1-6, as defined immediately hereinbelow, are present in all 50 genes. This includes motifs 1-3 (SEQ ID NOs 48-50, respectively), which represent the two AP2 domains and a sequence linking the two domains (linker sequence). Motif 4, with the consensus sequence of PK[L/V][E/A][D/N]FLG (SEQ ID NO: 51) is amino-terminal to the two AP2 domains. Motif 5 (SEQ ID NO: 52) flanks the two AP2 domains on the carboxy terminal end of the polypeptides. Near the amino terminus of the polypeptides is motif 6, with the consensus sequence of NWL[G/S]FSLSP (SEQ ID NO: 53).

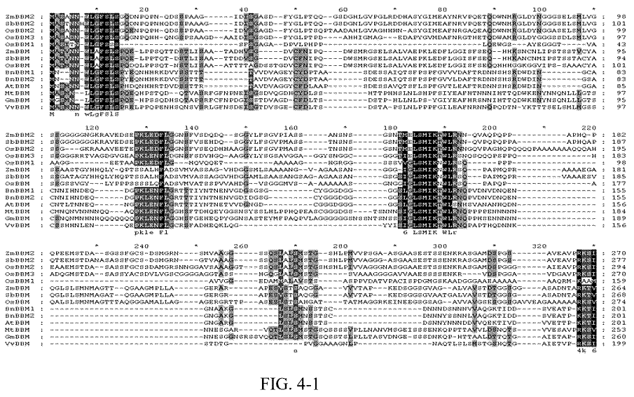

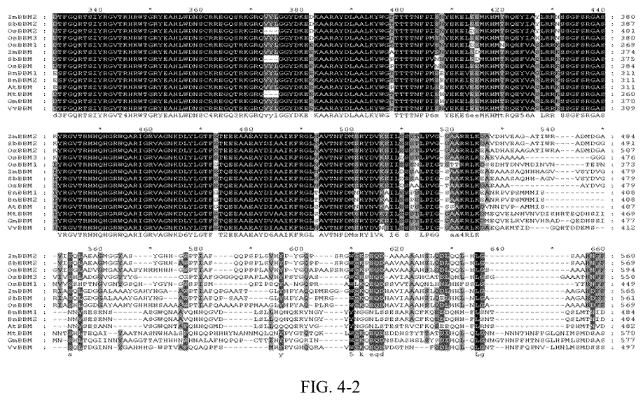

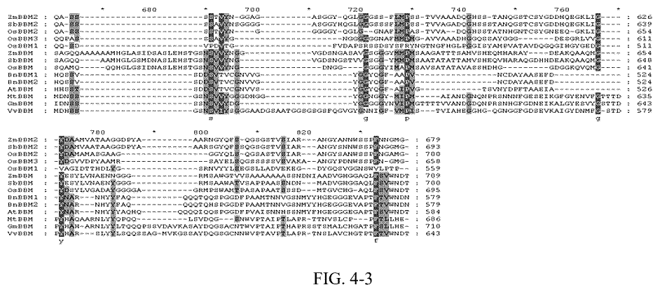

There were motifs that were relatively specific for the BBM subgroup of the homologous sequences (referred to herein as BBM polypeptides). An alignment of the BBM polypeptides can be found in FIG. 4. Motif 7 is found in all BBM polypeptides at the amino terminus of the polypeptide and has the consensus sequence of [G/E]LSMIK[T/N]WLR (SEQ ID NO: 54). Another motif that is present in all of the BBM polypeptides except for the polypeptides from Brassica and from Arabidopsis, is Motif 10. Motif 10 has the consensus sequence of WCK[Q/P]EQD (SEQ ID NO: 57) and is located downstream of the AP2 domains.

There are three more motifs specific to the BBM group of polypeptides, including Motif 15 (SEQ ID NO: 59) which appears only in BBM orthologs, but not in the monocot BBM2 polypeptides; a monocot specific motif (Motif 19; SEQ ID NO: 60); and a general BBM specific motif (Motif 14; SEQ ID NO: 58), which appears in BBM homologs except for the Brassica and legume branch.

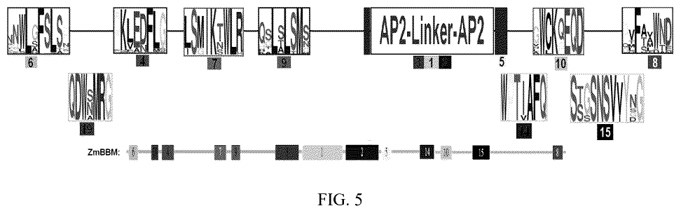

FIG. 5 provides a summary of the motif structure of the BBM homologs. The amino terminal motifs 4 and 6 and the AP2 flanking motif 5 distinguish the BBM homologous sequences from other two AP2 domain-containing homologs, such as WRI, AP2, and RAP2.7. Therefore, motifs 1-6 can be considered as core BBM/PLT family motifs. Many subgroups of the BBM/PLT family (BBM, PLT1/2, AIL1, and ANT) also have a carboxy-terminal motif (motif 8; SEQ ID NO: 55) and the third amino terminal motif (motif 9; SEQ ID NO: 56).

The BBM polypeptides all have one additional motif (motif 7; SEQ ID NO: 54) in the amino terminus, and all but the Brassica and Arabidopsis BBM homologs have an AP2 downstream motif (motif 10; SEQ ID NO: 57). Some other BBM/PLT family members (e.g., monocot AIL1) may have a similar motif as motif 7, but none of them also have motif 9. Motif 10 appears only in BBM polypeptides. In summary, the MEME predicted motifs 1-10 can be regarded as BBM polypeptide motifs. All monocot BBM polypeptides (corn, sorghum, and rice) also have motif 14, 15, and 19 (see FIG. 3). Some dicot BBM polypeptides and the second monocot BBM group (BBM2) have one or two of these motifs, but none have all three motifs.

### Example 9. Use of Maize BBM and WUS2 to Increase Transformation in Rice

a. Oryza sativa L. ssp. Indica

Mature and immature Indica embryos were transformed using Agrobacterium with a T-DNA comprising the PHP46911 plasmid (control for immature embryos; see immediately hereinbelow for a description), the PHP32269 plasmid (control for mature embryos; see immediately hereinbelow for a description), or PHP35648.

$$PHP 46911 : RB - CaMV 35 S \text{::} Hyg \text{::} Nos term + Ubi - FRT 1 \text{::} Zs - yellow 1 \text{::} Pin II - FRT 87 - LB$$

$$PHP 32269 : RB - Ubi \text{::} PMI \text{::} PinII + Ubi \text{::} mo - PAT ∼ Zs - yellow 1 \text{::} PinII - LB$$

$$( PMI = phosphomannose isomerase )$$

i. Immature Embryo Transformation

Immature embryos of proprietary Indica strain 851G were transformed using the methods disclosed in International Application Publication No. WO/1995/06722 and Hiei and Komari (2006) Plant Cell, Tissue and Organ Culture 85:271-283, each of which is herein incorporated by reference in its entirety. Results are shown hereinbelow in Table 4.

TABLE 4
Transformation events in Oryza sativa L. ssp. Indica 851G immature
embryos infected with Agrobacterium containing PHP35648.

| Embryo No. | No. of Pieces/Embryo | Total No. of Events/Embryo |
|---|---|---|
| 1 | 2 | 1 (100%) |
| 2 | 3 | 1 (100%) |
| 3 | 6 | 3 (300%) |
| 4 | 2 | 2 (200%) |
| 5 | 3 | 3 (300%) |
| 6 | 3 | 2 (200%) |
| 7 | 6 | 2 (200%) |
| 8 | 7 | 2 (200%) |
| 9 | 11 | 6 (600%) |
| 10 | 3 | 3 (300%) |
| 11 | 5 | 3 (300%) |
| 12 | 3 | 3 (300%) |
| TOTAL | 54 | 31 (258%) |

In total, infection of 12 immature Indica embryos with Agrobacterium containing the PHP35648 plasmid resulted in 31 transformation events, a transformation frequency of 258% events/embryo. The 31 events were derived from 54 pieces of embryo, for a transformation frequency of 5700 events/embryo pieces. On the other hand, the infection of nine embryos with Agrobacterium containing PHP46911 resulted in only one single transformation event and an overall transformation frequency of 1100.

ii. Mature Embryo Transformation

Mature embryos of Indica strains IRV95 and 851G were transformed using the following protocol. Healthy rice seeds were dehusked and soaked in 50 ml of sterile water with a drop of Tween 20 for 5 minutes. The seeds were sterilized with 7500 ethanol for 2-3 minutes, followed by a soak in 50 ml sodium hypochlorite and a drop of Tween 20 for 15-20 minutes. The seeds were rinsed and then callus was initiated in callus induction medium (4.3 g/l MS salts, 10 ml/l B5 vitamins (100×), 2 mg/l 2,4-D, 500 mg/l L-proline, 30 g/l sucrose, 0.3 g/casein hydrolysate, 3 g/l Gelrite (added after bringing to volume with D-I H₂O and adjusting pH to 5.8) under continuous light at 32° C. for 12 days.

Established callus was transformed using Agrobacterium by incubating the callus with the Agrobacterium for 10-15 minutes. The Agrobacterium solution was then decanted and 12-15 seeds were placed onto a filter paper disk that had been pre-moistened with 0.5 ml of AAM medium (50 ml/l AA macro elements (20×), 10 ml/l AA microelements (B5 microelements; 100×), 10 ml/l AA vitamins (B5 vitamins; 100×), 5 ml/l Fe-EDTA-B5 (200×), 1 mg/L 2,4-D, 100 ml/l amino acids, 68.5 g/l sucrose, 36 g/l glucose, 500 mg/l cas amino acid at pH 5.2) containing 50 M acetosyringone. The seeds and pre-moistened filter papers were cultured in the dark at 21° C. for 72 hours in ACCM medium (4.3 g/l MS salts, 10 ml/l B5 vitamins (100×), 2 mg/l 2,4-D, 20 g/l sucrose, 10 g/l glucose, 0.5 g/l casein hydrolysate, 3 g/l Gelrite (added after bringing to volume with D-I H₂O and adjusting pH to 5.2) containing 200 M acetosyringone. The calli were washed and then transferred to resting ASM medium (100 ml/l 580S major salts (10×), 10 ml/l 580S minor salts (100×), 5 ml/l 580S FeETDA-L (200×), 5 ml/l 580S vitamins (200×), 100 mg/l myo-inositol, 300 mg/l casein hydrolysate, 30 g/l maltose, 2 mg/l 2,4-D, 500 mg/l L-proline, 0.5 g/l MES buffer, 8 g/l agar (added after bringing to volume with D-I H₂O and adjusting pH to 5.8) containing 250 mg/l carbenicillin for 15 days. Following the 15 day incubation, the calli were transferred to selection medium (100 ml/l 580S major salts (10×), 10 ml/l 580S minor salts (100×), 5 ml/l 580S FeEDTA-L (200×), 5 ml/l 580S vitamins (200×), 100 mg/l myo-inositol, 300 mg/l casein hydrolysate, 30 g/l sucrose, 2 mg/l 2,4-D, 500 mg/l L-proline, 0.5 g/l MES buffer, 8 g/l agar (added after bringing to volume with D-I H₂O and adjusting pH to 5.8) with 200 mg/l carbenicillin and 2 mg/l bialaphos and subcultured every 15 days until transformation events arose.

The event was then desiccated on filter paper at 28° C. for 48 hours to excise the developmental genes. Desiccated events were identified based on the expression of Zs-yellow visualized under the microscope. The desiccated event was transferred to regeneration medium (100 ml/l N6 major salts (10×), 10 ml/l FeEDTA (100×), 10 ml/l B5 minor salts (100×), 10 ml/l B5 vitamins (100×), 1 mg/l 1-naphthalene acetic acid, 3 mg/l 6-benzyl amino purine, 30 g/L maltose, 0.3 g/l proline, 0.3 g/l vitamin assay casamino acids, 4 g/l agarose type 1, 30 mg/l glutamine (added after adjusting pH to 5.8 and sterilization) and grown at 32° C. under light. After 1-1.5 months, green shoots emerged from the callus and it was transferred to rooting medium (100 ml/l MS major salts (10×), 10 ml/l FeEDTA (100×), 10 ml/l MS minor salts (100×), 10 ml/l MS vitamins (100×), 2 mg/l indole-3-butyric acid, 15 g/l sucrose, 1 g/l vitamin assay casamino acids, 10×AA amino acid at pH 5.8). After another 15-20 days, the rooted plants are hardened in Y-medium (1.25 ml/l Stock A (9.14 g/100 ml ammonium nitrate (HIMEDIA RM5657)), 1.25 ml/l Stock B (4.03 g/100 ml sodium hydrogen phosphate (HIMEDIA 58282)), 1.25 ml/l Stock C (7.14 g/100 ml potassium sulfate (HIMEDIA 29658-4B)), 1.25 ml/l Stock D (8.86 g/100 ml calcium chloride (HIMEDIA C5080)), 1.25 ml/l Stock E (3.234 g/100 ml magnesium sulfate (HIMEDIA RM683)), 1.25 ml/l Stock F (15 mg/100 ml magnesium chloride tetra hydrate (HIMEDIA 10149), 6.74 mg/100 ml ammonium molybdate (HIMEDIA 271974), 9.34 mg/100 ml boric acid (SIGMA 136768), 0.35 mg/100 ml zinc sulfate helpta hydrate (HIMEDIA RM695), 0.31 mg/100 ml copper sulfate hepta hydrate (HIMEDIA C8027), 0.77 mg/100 ml ferric chloride hexa hydrate (SIGMA 236489), 119 mg/100 ml citric acid monohydrate (HIMEDIA C4540)) at pH 5.2.

Results are shown hereinbelow in Table 5.

TABLE 5
Transformation events in Oryza sativa L. ssp. Indica 851G and IRV95 mature
embryo-derived callus.

| | | | | | | | % of |
|---|---|---|---|---|---|---|---|
| | | | No. of | | | No. of | regenerated |
| Seed | | | infected | No. of | % of | events | events/infected |
| No | Variety | Construct | calli | events | events | regenerated | calli |
| 1 | 851G | PHP35648 | 100 | 8 | 8.00% | 5 | 5.00% |
| | | PHP32269 | 50 | 1 | 2.00% | 0 | 0.00% |
| 2 | 851G | PHP35648 | 130 | 18 | 13.85% | N/Aª | N/A |
| | | PHP32269 | 50 | 1 | 2.00% | N/A | N/A |
| 3 | IRV95 | PHP35648 | 128 | 20 | 15.63% | N/A | N/A |
| | | PHP32269 | 50 | 1 | 2.00% | N/A | N/A |

^{a}N/A: data not available; calli are currently being dessicated, so no data on number or percentage of regenerated events are available

b. Nipponbare Rice (cv. Kitake)

Callus was initiated from mature embryos of Oryza sativa, var. Nipponbare, cv. Kitake, and established callus was transformed using Agrobacterium strain LBA4404 containing UBI::ZmBBM::PinII and NOS PRO::ZmWUS2::PinII between the T-DNA borders. Callus culture medium for rice consisted of N6 salts, Eriksson's vitamins, 0.5 mg/l thiamine, 2 mg/lo 2,4-D, 2.1 g/l proline, 30 g/l sucrose, 300 mg/l casein hydrolysate, 100 mg/l myo-inositol, and 3 g/l gelrite at pH 5.8. Five days after Agro-infection, callus was observed under an epifluorescent dissecting microscope. For calli that were transformed with UBI::ZS-GREEN::PinII alone, all the visible fluorescent foci were single cells, with a few possible 2-4 cell foci. When callus was transformed with NOS::ZmWUS2::PinII+UBI::ZmBBM::PinII+UBI::ZS-GREEN::PinII and observed 5 days later, numerous rapidly-growing, green-fluorescent, multicellular colonies were present.

### Example 10. The Rice, Sorghum and Grape BBM Genes Increase Transformation Frequency in Maize

Growth assays were performed to test whether BBM genes from various species would stimulate growth in maize. For these experiments, 10-13 DAP embryos of the genotype PH581 were bombarded with a first plasmid containing a UBI PRO::moPAT˜GFP::pinII expression cassette plus either a plasmid containing 35S::GUS::pinII (control treatment) or a BBM gene driven by the ubiquitin promoter. To attach the DNAs to gold particles, a 25 μl aliquot of 0.6 m particles (0.01 mg/μl) was added to fresh tubes before attaching DNA. To attach uncoated DNA, the particles were pulse-sonicated, then 500 ng of each DNA (in 5 μl water) was added, followed by mixing (pipetting up and down a few times with a Pipetteman). Then 2.5 μl of TFX-50 was added, and the solution was placed on a rotary shaker for 10 minutes. After centrifugation at 10,000 g for 1 minute, the supernatant was removed, and the particles were resuspended in 60 μl of EtOH, followed by a 10 minute incubation. The particles were spun briefly (i.e., 10 seconds), the supernatant removed, and 60 μl EtOH added. The solution was spotted onto macrocarriers and the gold particles onto which DNA had been attached were delivered into scutellar cells of 10-13 DAP immature embryos using a standard protocol for the DuPont PDS-1000 Helium Gun. After 4-5 weeks on culture medium, the embryos were examined and the number of GFP-expressing multicellular colonies were counted.

a. OsBBM

Based on the rice BBM genomic sequence (SEQ ID NO: 117), TIGR software was used to predict intron splicing and the resultant cDNA sequence (OsBBM (MOD1) is set forth in SEQ ID NO: 118). A plasmid containing an expression cassette for the rice BBM (MOD1) gene (UBI PRO::OsBBM (MOD1)::PinII) was co-delivered with UBI::moPAT˜GFP::PinII into 13 DAP PH581 immature embryos using the particle gun. When the UBI PRO::moPAT˜GFP::pinII cassette was introduced with 35S::GUS, few multicellular growing sectors were observed (see Tables 6-10). When UBI::ZmBBM::PinII was introduced along with UBI::moPAT˜GFP::PinII, a stimulation of growth was observed as indicated by the total number of growing multicellular colonies observed as well as the number of embryos with multiple growing colonies. Despite many conserved amino acid motifs between the encoded maize protein and the protein encoded by the predicted OsBBM (MOD1) cDNA, when the rice expression cassette UBI::OsBBM (MOD1)::PinII, was introduced along with the moPAT˜GFP cassette, no stimulation of growth was observed relative to the control treatment (35S::GUS) (see Tables 6 and 7). Based on a comparison of the maize and rice MOD1 amino acid sequences, and a more careful analysis of the rice genomic sequence, it was determined that the TIGR software failed to predict the splicing around a 9-bp exon that encodes the amino acids VYL in the first AP2 domain. Upon including this 9 bp exon in a re-synthesized rice cDNA (OsBBM (MOD2); set forth in SEQ ID NO: 120), and introducing this in the expression cassette UBI::OsBBM (MOD2)::PinII, a growth stimulation similar to that observed for the maize BBM gene was observed (Table 7, 8, 9 and 10).

TABLE 6
Number of green-fluorescent multicellular colonies six weeks after bombardment
with UBI::moPAT~GFP plus the plasmid indicated in each treatment.

| | | | | | | | | | | | | Total |
|---|---|---|---|---|---|---|---|---|---|---|---|---|
| | | | | | | | | | | | | Number of |
| | GFP + Colonies/Bombarded Embryo | | | | | | | | | | | Multicellular |
| TRT | 0 | 1 | 2 | 3 | 4 | 5 | 6 | 7 | 8 | 9 | 10 | Colonies |
| 35S::GUS | 44 | | | | | | | | | | | 0 |
| UBI::ZmBBM | 15 | 10 | 5 | 4 | 3 | 1 | 1 | | | 1 | | 25 |
| UBI::OsBBM | 42 | | | | | | | | | | | 0 |
| (MOD1) | | | | | | | | | | | | |
| OLE::ZmBBM | 14 | 16 | 6 | 1 | | | | | | | | 14 |
| OLE::ZmANT | 44 | | | | | | | | | | | 0 |

TABLE 7
Number of green-fluorescent multicellular colonies five weeks after bombardment
with UBI::moPAT~GFP plus the plasmid indicated in each treatment.

| | | | | | | | | | | Total Number of |
|---|---|---|---|---|---|---|---|---|---|---|
| | GFP + Colonies/Bombarded Embryo | | | | | | | | | Multicellular |
| TRT | 0 | 1 | 2 | 3 | 4 | 5 | 6 | 7 | 8 | Colonies |
| 35S::GUS | 70 | 5 | 0 | 3 | | | | | | 8 |
| UBI::ZmBBM | 19 | 24 | 17 | 7 | 3 | 1 | | | | 52 |
| UBI::OsBBM | 70 | 4 | 2 | | | | | | | 6 |
| (MOD1) | | | | | | | | | | |
| UBI::OsBBM | 28 | 29 | 11 | 3 | 1 | 1 | | | | 45 |
| (MOD2) | | | | | | | | | | |
| OLE::ZmBBM | 28 | 24 | 11 | 9 | 2 | 1 | 2 | | | 49 |
| OLE::ZmANT | 55 | 16 | 1 | 1 | 1 | | | | | 19 |

TABLE 8
Number of green-fluorescent multicellular colonies five weeks
after bombardment with UBI::moPAT~GFP plus the plasmid
indicated in each treatment.

| | | | | | | | | Total |
|---|---|---|---|---|---|---|---|---|
| | Number of GFP + | | | | | | | Number of |
| | Colonies per Scored Embryo | | | | | | | Multicellular |
| Treatment | 0 | 1 | 2 | 3 | 4 | 5 | 6 | Colonies |
| 35S::GUS | 70 | 5 | 0 | 3 | 0 | 0 | 0 | 8 |
| UBI::ZmBBM | 19 | 24 | 17 | 7 | 3 | 1 | 0 | 52 |
| UBI::OsBBM | 28 | 29 | 11 | 3 | 1 | 1 | 0 | 73 |
| (MOD2) | | | | | | | | |
| OLE::ZmBBM | 28 | 24 | 11 | 9 | 2 | 1 | 2 | 49 |
| OLE::ZmANT | 55 | 16 | 1 | 1 | 1 | 0 | 0 | 19 |

TABLE 9
Number of green-fluorescent multicellular colonies five weeks
after bombardment with UBI::moPAT~GFP plus the
plasmid indicated in each treatment.

| | | | | | | | | Total |
|---|---|---|---|---|---|---|---|---|
| | Number of GFP + | | | | | | | Number of |
| | Colonies per Scored Embryo | | | | | | | Multicellular |
| Treatment | 0 | 1 | 2 | 3 | 4 | 5 | 6 | Colonies |
| 35S::GUS | 61 | 6 | 1 | 0 | 0 | 0 | 0 | 7 |
| UBI::ZmBBM | 21 | 29 | 12 | 3 | 2 | 0 | 0 | 46 |
| UBI::OsBBM | 27 | 29 | 5 | 2 | 0 | 1 | 0 | 37 |
| (MOD2) | | | | | | | | |
| UBI::VvBBM | 32 | 21 | 6 | 0 | 1 | 0 | 0 | 28 |
| UBI::ZmBBM | 9 | 36 | 13 | 6 | 3 | 0 | 10 | 58 |
| (genomic) | | | | | | | | |

TABLE 10
Number of green-fluorescent multicellular colonies five weeks
after bombardment with UBI::moPAT~GFP plus the plasmid
indicated in each treatment.

| | | | | | | | | | | | | Total |
|---|---|---|---|---|---|---|---|---|---|---|---|---|
| | | | | | | | | | | | | Number of |
| | GFP + Colonies/Scored Embryo | | | | | | | | | | | Multicellular |
| TRT | 0 | 1 | 2 | 3 | 4 | 5 | 6 | 7 | 8 | 9 | 10 | Colonies |
| 35S::GUS | 80 | 11 | | | | | | | | | | 11 |
| UBI::ZmBBM | 43 | 28 | 13 | 5 | 2 | | | | | | | 48 |
| UBI::OsBBM | 45 | 32 | 11 | 3 | | | | | | | | 46 |
| (MOD2) | | | | | | | | | | | | |
| UBI::SbBBM | 81 | 10 | | | | | | | | | | 10 |
| (MOD1) | | | | | | | | | | | | |

b. SbBBM

Based on the sorghum genomic BBM sequence (SEQ ID NO: 69), TIGR software was used to predict intron splicing and the resultant cDNA sequence (SbBBM (MOD1) is set forth in SEQ ID NO: 3). A plasmid containing an expression cassette for the sorghum BBM (MOD1) gene (UBI PRO::SbBBM (MOD1)::PinII) was co-delivered with UBI::moPAT˜GFP::PinII into 13 DAP PH581 immature embryos using the particle gun. When the UBI PRO::moPAT˜GFP::PinII cassette was introduced with 35S::GUS, few multicellular growing sectors were observed (see Table 10). Unlike UBI::ZmBBM and UBI::OsBBM (MOD2), which in this experiment produced a similar positive growth stimulation, UBI::SbBBM (MOD1)::PinII failed to simulate growth. Assuming there was some unknown defect in the SbBBM (MOD1) synthetic cDNA, the sorghum genomic BBM was cloned using PCR and sequenced to verify fidelity. In an earlier experiment, the maize genomic BBM (SEQ ID NO: 116) was placed behind the UBI promoter and when compared to the UBI::ZmBBM cDNA construct it produced a similar degree of growth stimulation (Table 9). Using the genomic sorghum clone [UBI::SbBBM (GEN)], a similar level of growth stimulation was also observed (Tables 11 and 12).

TABLE 11
Number of green-fluorescent multicellular colonies five weeks
after bombardment with UBI::moPAT~GFP plus the plasmid
indicated in each treatment.

| | | | | | | | | | | Total |
|---|---|---|---|---|---|---|---|---|---|---|
| | | | | | | | | | | Number of |
| | GFP + Colonies/Scored Embryo | | | | | | | | | Multicellular |
| Treatment | 0 | 1 | 2 | 3 | 4 | 5 | 6 | 7 | 8 | Colonies * |
| 35S::GUS | 57 | 3 | | | | | | | | 3 |
| UBI:VvBBM- | 57 | 3 | | | | | | | | 3 |
| NoVYL | | | | | | | | | | |
| UBI:VvBBM | 36 | 15 | 4 | 1 | 1 | 1 | | | | 22 |
| UBI:SbBBM | 10 | 19 | 11 | 11 | 4 | 3 | 2 | | | 50 |
| (Genomic) | | | | | | | | | | |
| UBI:ZmBBM | 12 | 18 | 8 | 10 | 4 | 3 | 1 | | 1 | 45 |

TABLE 12
Number of green-fluorescent multicellular colonies five weeks
after bombardment with UBI::moPAT~GFP plus the plasmid
indicated in each treatment.

| | | | | | | | Total |
|---|---|---|---|---|---|---|---|
| | | | | | | | Number of |
| | GFP + Colonies/Scored Embryo | | | | | | Multicellular |
| TRT | 0 | 1 | 2 | 3 | 4 | 5 | Colonies |
| 35S::GUS | 60 | | | | | | 0 |
| UBI::ZmBBM | 19 | 18 | 11 | 7 | 4 | 1 | 41 |
| UBI::SbBBM | 20 | 15 | 14 | 6 | 5 | | 60 |
| (Genomic) | | | | | | | |
| UBI::VvBBM | 46 | 11 | 3 | | | | 14 |
| UBI::VvBBM- | 60 | | | | | | 0 |
| No VYL | | | | | | | |

c. VvBBM

A nucleotide sequence was derived that provided good codon usage for maize, but expressed the amino acid sequence of a grape BBM (VvBBM; SEQ ID NO: 5). A plasmid containing an expression cassette for a synthetic grape BBM gene (UBI PRO::VvBBM::PinII) was co-delivered with UBI::moPAT˜GFP::PinII into 10 DAP PH581 immature maize embryos using the particle gun. When the UBI PRO::moPAT˜GFP::PinII cassette was introduced alone, no (Table 12) or very few (Tables 9 and 11) multicellular growing sectors were observed. When UBI::VvBBM::PinII+UBI::moPAT˜GFP::PinII were co-delivered, numerous RFP+ multicellular colonies were observed growing on the surface of bombarded embryo after 4 weeks. As with growth stimulation by the maize, rice and sorghum BBM genes, the growth stimulation imparted by the UBI::VvBBM::PinII cassette was manifested by an increase in the overall number of multicellular colonies, and also an increase in the number of multicellular colonies growing on single embryos (see Tables 9, 11 and 12). When a construct comprising the VvBBM sequence, in which the 9-bp sequence encoding VYL in the AP2 domain was removed, was introduced into maize, there was no observed growth stimulation (Tables 11 and 12), similar to the observations made with the rice BBM gene lacking this same exon.

d. Maize ANT Gene

The following constructs were used for comparison: OLE PRO::ZmBBM::pinII, and OLE PRO::ZmANT::pinII. The nucleotide and amino acid sequences of ZmANT are set forth in SEQ ID NOs: 66 and 67. Each of these plasmids was co-delivered with UBI::moPAT˜GFP::pinII into 10 or 13 DAP PH581 immature embryos using the particle gun. When the UBI PRO::moPAT˜GFP::pinII cassette was introduced alone, no (Table 6) or few multicellular growing sectors (Tables 7 and 8) were observed. When OLE::ZmBBM::pinII+UBI::moPAT˜GFP::pinII were co-delivered, a substantial increase in the number of embryos with GFP+ multicellular colonies were observed growing on the surface of each bombarded embryo after 5 weeks (i.e. relative to the control treatment). In addition, the number of embryos supporting multiple GFP+ colonies increased. Embryos co-bombarded with OLE::ZmANT::pinII+UBI::moPAT::pinII appeared identical (Table 6, with no multicellular colonies in either treatment) or similar to the control treatment (FIGS. 6 and 7, with only a 2-fold increase in colony formation and numerous single GFP+ cells (indicating only transient expression but no division) and a reduced number of GFP+ colonies relative to the BBM treatment. In a second experiment with the same treatments (control with no BBM or ANT, Ole::BBM or Ole::ANT), out of 44 embryos shot per treatment, the control and ANT treatments produced no multicellular GFP+ colonies after 3 weeks while the BBM treatment produced 14 colonies.

### Example 11. Expression of the Maize BBM and WUS Genes Improves Transformation in Sorghum

Agrobacterium tumefaciens LBA4404 and a super-binary vector constructed with pSB1 and pSB11 (Komari et al. (1996) Plant J 10:165-174; Thompson et al. (1987) EMBO J 6:2519-2523) can be used for sorghum transformation (Zhao (2006) In “Agrobacterium Protocols,” vol. 1, Kan Wang, ed. Hamana Press, Totowa, NJ; U.S. Pat. No. 6,369,298; and International Application Publication No. WO 98/49332). The super-binary vector contained a selectable marker gene, bar (Chalfie et al. (1994) Science 263:802-805) and a visible marker gene, such as red fluorescent protein (RFP), yellow fluorescent protein (YFP), or intron-GFP (Jefferson et al. (1986) Proc Natl Acad Sci USA 83:8447-8451).

Minimal AB media included 50 ml/l Stock A, 50 ml/l Stock B, 5 g/l glucose, 9 g/l Phytagar. For the Agrobacterium strain used in this protocol, 50 mg/l spectinomycin is added after autoclaving. Stock A included 60 g/l K₂HPO₄, and 20 g/l NaH₂PO₄, pH 7.0. Stock B is 20 g/l NH₄Cl, 6 g/l MgSO₄ 7H₂O, 3 g/l KCl, 0.2 g/l CaCl₂), and 0.5 g/l FeSO₄H₂O. YP medium contained 5 g/l yeast extract, 10 g/l peptone, 5 g/l NaCl, and 15 g/l Bacto-agar. For the Agrobacterium stain used in this protocol, 50 mg/l spectinomycin was added after autoclaving.

PHI-I media included 4.3 g/l MS salts (GIBCO BRL catalog no. 11117-874), 0.5 mg/l nicotinic acid, 0.5 mg/l pyridoxine HCl, 1 mg/l thiamine HCl, 0.1 g/l myo-inositol, 1 g/l vitamin assay casamino acids, 1.5 mg/l 2,4-D, 68.5 g/l sucrose, 36 g/l glucose, pH 5.2. 100 μM acetosyringone is added before using.

PHI-T media included PHI-I with sucrose reduced to 20 g/l and glucose reduced to 10 g/l, 2, 4-D increased to 2 mg/l, and with 0.5 g/l MES buffer, 0.7 g/l L-proline, 10 mg/l ascorbic acid, 100 μM acetosyringone and 8 g/l agar, pH 5.8 added.

PHI-U media included PHI-T without glucose and acetosyringone, and with 1.5 mg/l 2,4-D, 100 mg/l carbenicillin, and 5 mg/l PPT (glufosinate-HN4) added.

PHI-RF media included 4.3 g/L MS salts (GIBCO BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.49 μM cupric sulfate, 0.5 mg/L zeatin (Sigma Z-0164), 1 mg/L IAA, 26.4 g/L ABA, 0.1 mg/L thidiazuron, 60 g/L sucrose, 3 mg/L bialaphos, 100 mg/L carbenicillin, and 8 g/L agar, pH 5.6.

PHI-Z media included 2.15 g/L MS salts, 2.5 ml/L MS vitamin mix, 20 g/L sucrose, and 3 g/L gelrite, pH 5.6

Suspension for immature embryo infection consisted of 100 μM acetosyringone in PHI-I medium (pre-warmed to room temperature). Bacteria were scraped off a working plate with a sterile bacteria loop and placed in PHI-I with 100 μM acetosyringone. The suspension was vigorously vortexed to break clumps and form a uniform suspension as determined by visual inspection. 1 ml of Agro-suspension was taken to measure optical density at 550 nm. The suspension was diluted with PHI-I plus 100 μM acetosyringone to 10⁹ cfu/ml (OD at 0.7).

Sorghum plants were grown under greenhouse, growth chamber, or field conditions. Healthy sorghum plants were always important for a successful transformation. Immature panicles were harvested 9-13 days post-pollination depending on the growing conditions. The size of immature zygotic embryos used in transformation ranged from 0.8 to 2.5 mm in length. Immature kernels were removed from the panicles and sterilized with 50% bleach and 0.1% Tween-20 for 30 min. with vacuum, then the kernels were rinsed three times with sterile water. The kernels were kept in sterile water before isolating embryos. Embryos were aseptically dissected from each sterilized sorghum kernel and placed in a 2-ml microtube containing 2 ml PHI-I with 100 μM acetosyringone. Usually, about 100 embryos were placed in each tube.

PHI-I liquid medium was removed from the tube comprising the embryos with a 1 ml micropipettor and replaced with 1 ml of the Agrobacterium suspension. The tube was gently inverted a few times to mix well and incubated 5 minutes at room temperature. The Agrobacterium suspension was removed from the tube with a 1 ml micropipettor. The embryos were scraped from the tube using a sterile spatula. Immature embryos were transferred to a plate of PHI-T medium in a 100×15 mm Petri dish. The embryos were oriented with embryonic axis down on the surface of the medium. These embryos were incubated at 21-25° C. in the dark for 3 days. The embryos were transferred to PHI-U minus PPT with the same orientation and incubated at 28° C. in the dark for 4 days.

The embryos were transferred to PHI-U medium and incubated at 28° C. in the dark for 2-3 weeks and were subcultured every two to three weeks for about 10-20 weeks to obtain enough callus for regeneration into plants.

These calli were transferred to PHI-RF medium and incubated at 28° C. in the dark for approximately 2-3 weeks to develop shoots. When shoots formed, these cultures were moved to a lighted culture room under conditions of 16 hours light (270 μE m⁻² sec⁻¹) and 8 hours dark at 25° C. Shoots (about 3-5 cm tall) were transferred to plastic boxes (10×9×10 cm) containing PHI-Z medium. These shoots were cultured under the same light and temperature conditions for 3-5 days. Each box contained shoots derived from a single embryo. When the plantlets reached about 8-10 cm tall with healthy roots, these plantlets were transferred to pots with Universal Mix (Strong-Lite, Seneca, IL61360) in the greenhouse.

Embryos were harvested from developing sorghum seed and transformed using Agrobacterium, delivering the PHP32371 T-DNA (see Example 4). As a control treatment, embryos were transformed with RB-Ubi::moPAT+Ubi:CFP-LB. Callus was selected on 3 mg/l bialaphos, and monitored for fluorescence to aid in identifying transgenic sectors. Sorghum transformation frequencies using Ubi:moPAT+Ubi:CFP averaged 0.5%. By comparison, in six experiments, a total of 393 embryos were transformed with PHP32371, producing an average transformation frequency of 18.3% (see Table 13). Callus from the first experiment (30 events from a starting total of 140 embryos) was used to test the desiccation-induced excision controlled by the Rab17 promoter, and subsequent plant regeneration. Twenty-one events were desiccated for three days on dry filter papers and then taken through the standard regeneration protocol. Fifteen of the 21 events produced a total of 81 plants, with multiple plants being regenerated for many of the individual events. Of these, 60% contained a single copy of the integrated DNA, and of the single copy events, 91% produced PCR results indicating complete excision of the genes encoding cell proliferation factors. From excised events, normal phenotype plants lacking FLP and WUS2 were readily regenerated.

TABLE 13
Transformation efficiencies after Agrobacterium-mediated
transformation with PHP32371.

| | | Number of | |
|---|---|---|---|
| Experiment | Number of | Transformation | Transformation |
| ID | Embryos | Events | Frequency (%) |
| 1 | 140 | 30 | 21.4 |
| 2 | 40 | 3 | 7.5 |
| 3 | 60 | 8 | 13.3 |
| 4 | 40 | 7 | 17.5 |
| 5 | 61 | 12 | 19.7 |
| 6 | 52 | 12 | 23.1 |
| Average | | | 18.3 |

### Example 12. Expression of the Maize BBM and WUS Genes Improves Transformation in Sugarcane

A developmental gene binary vector with the ZmBBM/ZmWUS2 gene cassette was compared with a standard vector containing moPAT plus either DsRED or YFP without the ZmBBM/ZmWUS2 gene cassette for transformation frequency using two Agrobacterium strains, AGL1 and LBA4404, in CP89-2376 and CP01-1372 sugarcane cultivars. The developmental gene binary vector contains Ubi::LoxP::CFP+Rab17Pro-attB1::Cre-attB2::PinII+Nos::ZmWUS2::PinII+Ubi::ZmBBM::PinII-LoxP::YFP+Ubi:: MOPAT::PinII. The Lox cassette containing CFP::Cre::WUS::BBM can be excised by Cre recombinase controlled by the Rab17 promoter. Callus tissues of both CP89-2376 and CP01-1372 cultivars were induced and maintained on DBC3 medium. Tissues were infected with Agrobacterium containing the developmental gene binary vector in 10 mM MgSO₄ plus 100 uM acetosyringone and then cocultivated with liquid DBC3(M5G) medium plus 100 uM acetosyringone on the filter paper in Petri dishes at 21° C. in the dark. Three days after cocultivation, the tissues were transferred to DBC3 containing 100 mg/L cefotaxime and 150 mg/L timentin for AGL1, and DBC3 containing 100 mg/L carbenicillin for LBA4404, and incubated at 26° C. (±1° C.) in the dark or dim light for 3-7 days. Afterwards, the tissues were transferred to the same media as the previous step plus 3 or 5 mg/L bialaphos. After two months from the initiation of the experiment, transformation frequency was calculated by the number of tissues showing CFP expression divided by the number of explants infected by Agrobacterium. Table 14 demonstrated that AGL1 was even more efficient in transformation than LBA4404 in both CP89-2376 and CPO1-1372. There was also a genotype difference in transformation frequency; CP89-2376 had much higher transformation frequencies than CPO1-1372 using either of the Agrobacterium strains.

AGL1 containing the developmental gene vector was also used to test sugarcane germplasm screening in another set of experiments using 5 different cultivars (CP96-1252, CPO1-1372, CP89-2376, CPCL97-2730 and HoCP85-845). Callus tissues of all 5 cultivars tested were induced and maintained on DBC3 medium and tissues were infected with AGL1 containing the developmental gene binary vector. The use of developmental genes dramatically increased transformation frequency in all 5 cultivars tested. Transformation frequencies in the most amenable cultivar, CP89-2376, using a standard binary vector averaged 116.7% (56/48) (Table 14). In contrast, an average transformation frequency in this cultivar from 5 experiments was >2,512.5% (>1,005 events/40 tissues infected) using the developmental gene binary vector. Similar results were obtained from the remaining 4 cultivars, CP96-1252, CPO1-1372, CPCL97-2730 and HoCP85-845; transformation frequencies ranged from 62.5% to 187.5% in these 4 cultivars while no transgenic events were obtained using the standard vector without the BBM/WUS gene cassette from these cultivars.

TABLE 14
Transformation frequency in sugarcane using the developmental
genes ZmBBM and ZmWUS2.

| Agrobacterium | | | | | | |
|---|---|---|---|---|---|---|
| | Binary | Sugarcane Cultivar | | | | |
| Strain | Vector | CP96-1252 | CP01-1372 | CP89-2376 | CPCL97-2730 | HoCP85-845 |
| AGL1 | DGª | n.t. ^{c} | 37.5% | n.t. | n.t. | n.t. |
| | | | (3/8) | | | |
| LBA4404 | DG | n.t. | 0% | n.t. | n.t. | n.t. |
| | | | (0/8) | | | |
| AGL1 | DG | n.t. | >1,250.0% | >6,250.0% | n.t. | n.t. |
| | | | (>100/8) | (>500/8) | | |
| LBA4404 | DG | n.t. | 12.5% | >1,500% | n.t. | n.t. |
| | | | (1/8) | (>120/8) | | |
| AGL1 | DG | n.t. | n.t. | 687.5% | n.t. | n.t. |
| | | | | (>55/8) | | |
| AGL1 | DG | n.t. | n.t. | >2,500% | 175.0% | n.t. |
| | | | | (>200/8) | (14/8) | |
| AGL1 | DG | 150.0% | 62.5% | >625.0% | 62.5% | n.t. |
| | | (12/8) | (5/8) | (>50/8) | (6/8) | |
| AGL1 | DG | n.t. | n.t. | >2,500% | n.t. | 187.5% |
| | | | | (>200/8) | | (15/8) |
| AGL1 | Std^{b} | 0% | 0% | 116.7% | 0% | 0% |
| | | (0/8) | (0/8) | (56/48) | (0/8) | (0/8) |

Each transformation treatment had 8 pieces of callus tissues 0.4-0.5 cm in size.
DGª: developmental gene vector with BBM/WUS gene cassette
Std^{b}: standard vector without BBM/WUS gene cassette
n.t. ^{c}: not tested

Transgenic callus tissues were desiccated on dry filter papers for three days to induce excision of the Lox cassette containing CFP::Cre::WUS::BBM by Cre recombinase driven by the Rab17 promoter. Excision was monitored by observing YFP expression on desiccated transgenic callus events by the presence of the UBI:loxP:YFP junction formed as a result of excision. Cre excision occurred at 83 of 87 transgenic events (95.4%) (Table 15). Plants from some transgenic events after excision are being regenerated on MSB plus 1 mg/L bialaphos and antibiotics.

TABLE 15
Excision efficiency of the BBM/WUS gene cassette
in transgenic sugarcane events by desiccation.

| Sugarcane | Agrobacterium | Binary | Excision Efficiency | |
|---|---|---|---|---|
| Cultivar | Strain | Vector | (%) | |
| CP89-2376 | AGL1 | DG^{a} | 93% | (40/43) |
| CP89-2376 | LBA4404 | DG | 100% | (25/25) |
| CP01-1372 | AGL1 | DG | 100% | (13/13) |
| CP01-1372 | LBA4404 | DG | 0% | (0/1) |
| CP89-2376 | AGL1 | DG | 100% | (5/5) |

| | | |
|---|---|---|
| Average | 95.4% | (83/87) |

DG^{a}: developmental gene vector with BBM/WUS gene cassette

### Example 13. Complementation of Separately Transformed BBM and WUS2 Genes

Nos::ZmWUS2::PinII and Rab17-attB1::CRE::PinII are integrated into the genome of an inbred maize plant. LoxP-UBI::BBM::PinII-LoxP+a trait gene operably linked to a promoter are re-transformed into the inbred as a single T-DNA. The BBM and WUS2 genes will complement each other, stimulating rapid growth only in the cells where both are present. BBM is then excised and normal fertile plants are regenerated. Later, the WUS2/CRE locus is segregated away from the genome.

### Example 14. Transformation of Mature Dried Maize Seed

Cell proliferation factors can be used to increase transformation and/or recovery frequencies in recalcitrant plants and/or target tissues, such as mature seed.

A T-DNA containing an excisable construct comprising a maize BBM and a maize WUS gene was constructed:

$$PHP 38333 : RB - Ubi - LoxP \text{::} CFP \text{::} PinII - attB 4 + Rab 17 Pro - attb 1 \text{::} Cre - attB 2 \text{::} PinII + Nos \text{::} ZmWUS 2 \text{::} PinII + Ubi \text{::} ZmBBM \text{::} PinII - LoxP \text{::} YFP \text{::} PinII + Ubi \text{::} moPAT \text{::} PinII - LB$$

As a control treatment, embryos were transformed with PHP32269: RB-Ubi::moPAT-YFP::PinII-LB.

The glycerol stock of a thymidine-auxotrophic mutant Agrobacterium strain LBA4404 with vector PHP38333, or the control vector were stored at −80° C. before use. A master plate was made by dipping an inoculation loop into a glycerol stock and streaking onto 12V solid medium with 50 mg/l thymidine in a 100×15 Petri dish (for PHP38333) or onto 12S solid medium with 50 mg/l spectinomycin (for the control plasmid). Plates were incubated (inverted) at 28° C. in the dark for 2-3 days to produce single colonies. Master plates were stored at 4° C. for up to 4 weeks and are used for initiating fresh culture for transformation. Several colonies were picked from the master plates and streaked onto 810F solid medium with 50 mg/l thymidine and incubated at 28° C., in the dark for 1 day and fresh Agrobacterium was used for transformation.

To make the Agrobacterium suspension, 20 ml of 700 liquid medium with 50 mg/l thymidine was added into a 50 ml snap cap tube. A stock solution of acetosyringone (AS) was added to achieve a final concentration of 200 uM and a stock solution of Silwet L-77 was added to achieve a final concentration of 0.04%. Agrobacterium was collected from a 1-day culture plate and suspended in the 700 liquid medium. The tube was vortexed until the Agrobacterium culture clumps were completely broken up and evenly dispersed throughout the solution. One ml of the suspension was transferred to a spectrophotometer tube and the OD of the suspension was adjusted to 0.7 at 550 nm by adding either more Agrobacterium or more of the same suspension medium.

Maize inbred line PHN46 was used as the initial genotype for transformation tests. Dry seeds were placed in a covered glass jar, in an 80% ethanol solution with stirring for 5 min. The ethanol was decanted and a 50% bleach solution with a few drops of the surfactant Tween-20 were added and seeds in the bleach solution were stirred for 30 min and washed three times with sterile water in a sterile flow hood. Surface sterilized seed were soaked in the sterile water for approximately 24 h at room temperature, which is sufficient to trigger germination. After 24 hours, the softened seeds were sterilized once again with a 50% bleach solution for 5 min, and then washed three times with sterile water in a sterile flow hood.

Mature embryos were dissected out of the softened and sterilized kernels. Each mature embryo was sliced into 3-4 thin sections by hand using a No. 10 surgical scalpel under the dissecting microscope. Each explant contained exposed leaf primordia, mesocotyl and root primordia regions. These regions on the embryo chips were the target area for T-DNA delivery during Agrobacterium-mediated transformation and contain cells that are culture responsive. Sliced explants were transferred into a 6-well culture plate containing 4 ml 700 liquid medium. About 45 explants were placed into each well for Agrobacterium infection.

Liquid medium in the 6-well plate was removed from the explants and replaced with 4 ml of prepared Agrobacterium suspension. The 6-well plate was transferred into a transparent polycarbonate desiccator container. The desiccator was covered and placed on a platform shaker rotating at a speed of 100 RPM and connected to an in-house vacuum system for 30 min. After infection, the Agrobacterium suspension was drawn off from the wells and the explants were transferred onto solid 7101 co-cultivation medium with 50 mg/l thymidine. The infected embryo explants on the solid medium were incubated at 21° C. in the dark for 3 days. The number of infected explants was recorded to later calculate transformation efficiencies.

To evaluate T-DNA delivery efficiency, both the control vector without genes encoding cell proliferation factors and the vector with the genes encoding cell proliferation factors were used to infect embryo explants. After 3d co-cultivation, all of the chips were transferred onto 605J medium for continuous culture. T-DNA delivery was evaluated around 5d after Agrobacterium infection. Transient expression of the color marker YFP (control vector) or CFP (test vector PHP38333) was a reliable indicator of the T-DNA delivery efficiency. In general, 30%-50% of the infected explants showed T-DNA delivery in the right target tissues or cells. Using an optimized infection medium and protocol, 70%-80% T-DNA delivery efficiency to the target area was achieved. Infected explants were sub-cultured to fresh medium every 3 weeks. After 6 weeks of culture, healthy, vigorously growing, embryogenic type I callus could be identified from those explants that had been infected with vector PHP38333 containing the genes encoding cell proliferation factors. These growing calli represented transformed events confirmed by the color marker (CFP) expression. Non-transformed tissues showed either no growth or very limited growth. Embryogenic type I callus were picked and transferred onto fresh culture medium to let the callus proliferate before plant regeneration (10-12 weeks). Transformation efficiency for PHP38333 at the callus level ranged from 12% to 20% calculated as the number events recovered per total number of infected explants (Table 16). Embryo explants that were infected with control vector PHP32269 also showed good T-DNA delivery based on transient YFP expression in the infected cells. However, these cells did not show significant proliferation and no healthy callus was formed during continuous culture.

TABLE 16
Transformation frequency of PHP38333 in PHN46 embryo chips.

| Experiment | Number of | Number of | Transformation |
|---|---|---|---|
| No. | Chips infected | CFP(+) Events | Frequency (%) |
| 1 | 137 | 23 | 16.8% |
| 2 | 134 | 19 | 14.2% |
| 3 | 149 | 20 | 13.4% |
| 4 | 140 | 25 | 17.9% |
| 5 | 148 | 18 | 12.2% |
| 6 | 137 | 26 | 19.0% |
| 7 | 129 | 27 | 20.9% |
| 8 | 136 | 20 | 14.7% |
| 9 | 137 | 21 | 15.3% |
| 10 | 147 | 24 | 16.3% |
| Total | 1393 | 223 | 16.0% |

Transformed callus tissues were treated with either one of the following two desiccation methods to induce excision of the genes encoding cell proliferation factors before plant regeneration.

1) Desiccation by natural air exchange: Transformed callus tissues were transferred to an empty 60 mm×25 mm Petri dish containing a piece of autoclaved glass filter paper and covered with a lid but not sealed. Petri dishes with callus tissues were placed into a culture box with a loose cover. The box was kept at 28° C. in the dark for 3 days.

2) Desiccation in chambers containing a saturated salt solution: Transformed callus tissues were transferred to an empty 60 mm×25 mm Petri dish containing a piece of autoclaved glass filter paper and covered with a lid. The Petri dishes with callus tissues were placed into a container with a tight sealing cover. A glass jar containing saturated (NH₄)₂SO₄ salt solution without a cover was placed in the container. The container was kept at 28° C. in the dark for 3 days (as the moisture in the air inside the container was absorbed by the saturated salt solution, the callus tissue gradually lost water and experienced desiccation stress).

After 3 days of desiccation treatment, the callus tissues were transferred to 289L regeneration media for 2-3 weeks in the dark. When shoots formed with a length of about 1-2 cm, callus tissues with shoots were transferred to hormone-free 272V medium for further development of shoots and roots in the light culture room. When plantlets had formed well-developed shoots and roots, plant regeneration efficiency was evaluated. The plant regeneration frequency (number of callus producing plants out of total number of callus events for plant regeneration) varied from 45% to 75% among 10 initial experiments. At this stage, leaf samples were collected from the plantlets derived from each callus event for molecular analysis. Detailed PCR analyses were performed to determine the copy number of transgenes as well as to confirm that the genes encoding proliferation factors were excised and were not present in the regenerated transgenic plants.

Based on the molecular analysis of 316 T0 plants from 162 events, about 60% of the transgenic plants contain a single copy of the transgenes. These single-copy transgenic plants showed very efficient excision of the genes encoding cell proliferation factors from the desiccation-treatment-induction (see results in Table 17). In general, plants with complete excision of genes encoding cell proliferation factors displayed normal phenotype in the tube and also in later developmental stages in the greenhouse. In contrast, T0 plants in which excision did not occur (or where it was incomplete) displayed an abnormal phenotype, such as thickened roots.

Based on PCR analysis results, chimeric or incomplete excision TO plants can be eliminated and only complete-excision (free of genes encoding cell proliferation factors) events were sent to the greenhouse.

TABLE 17
Analysis of T0 plants for excision of genes
encoding cell proliferation factors.

| Number of | | |
|---|---|---|
| Events/T0 Plants | Single Copy | Complete Excision |
| 162 (Events) | 103 (63.6%) | 94 (91.3%) |
| 316 (Plants) | 189 (59.8%) | 173 (91.5%) |

### Example 15. Transformation of Leaf Tissues

a. Preparation of Agrobacterium and Maize Leaf Explants

Agrobacterium suspensions were prepared as described in Example 14. Pioneer maize inbred lines PHN46, PHR03 and PHEJW were used as the initial genotypes for transformation tests. Dry seed was sterilized and imbibed overnight as described above.

Sterilized seeds were placed onto 272V solid medium for direct germination. Alternatively, mature embryos were dissected from softened and sterilized seeds and placed onto 272V solid medium for faster germination. Plates with seeds or isolated embryos were placed in a culture box and incubated at 28° C. in the dark for 3-7 days. Shoot segments of about 2-3 cm long above the first leaf base node of the seedling were excised under sterile conditions. The coleoptile was removed and the leaf fragment was split longitudinally first, then cross-dissected into smaller segments (0.5 to 2 mm). Alternatively, the 2-3 cm-long segment above the first leaf base node of the seedling was simply diced with the scalpel to produce small leaf segments. Small leaf segments were transferred into a 6-well culture plate containing 4 ml of 700 liquid medium.

Liquid medium in the 6-well plate with leaf pieces were drawn off and replaced with 4 ml prepared Agrobacterium suspension. The 6-well plate was transferred into a transparent polycarbonate desiccator container. The desiccator was covered and placed on a platform of the shaker with a speed of 100 RPM and connected to an in-house vacuum system for 15 min. After infection, the Agrobacterium suspension was drawn off from the wells and the leaf tissues were transferred onto solid 7101 co-cultivation medium with 50 mg/l thymidine and were incubated at 21° C. in the dark for 3 days.

After 3d co-cultivation, all of the leaf tissues were transferred to 13152C culture medium. T-DNA delivery was evaluated about 5d after Agrobacterium infection. Transient expression of the color marker YFP (control vector) or CFP (test vector PHP38333) was a reliable indicator of the T-DNA delivery efficiency. 10%-25% of infected leaf segments showed multiple fluorescent cells along the cut edges or surface of leaf segments in all three inbred lines tested. Infected leaf tissues were sub-cultured every 2 weeks. After 6-8 weeks of culture, stable transformed callus events could be identified. The transgenic nature of these stable callus events was indicated by the expression of the fluorescent gene. Callus events with significant proliferation were subjected to desiccation treatment, and transferred onto regeneration medium for 2-4 weeks. Stable transgenic plantlets were regenerated from two tested maize inbreds, PHN46 and PHR03. Results from numerous experiments clearly demonstrated that stable transgenic plants could be produced form transformation of seedling tissue by using the vector that expresses the genes encoding cell proliferation factors. Leaf tissues infected with the control vector also showed good T-DNA delivery based on transient YFP expression, but the infected cells did not exhibit any subsequent proliferation and no stable callus events were identified from this treatment.

### Example 16. The Utilization of Cell Proliferation Factors for Enhancing Chloroplast Transformation

For tobacco and a number of other species, leaves are a preferred target for chloroplast transformation. Cell proliferation factors are used to trigger a tissue culture response from leaves of maize and other species. For boosting chloroplast transformation, cell proliferation factor genes under the control of inducible promoters are introduced into the species of interest by standard nuclear transformation protocols. Events that contain the transgene are characterized for expression of the inducible cell proliferation factor genes. For example, leaves of maize from plants transformed with the cell proliferation factor genes under the control of the tetracycline-repressor system are placed on medium containing appropriate concentrations of doxycyline. The doxycyline then activates the cell proliferation factor genes and thereby induces an embryogenic tissue culture response. The leaves are maintained on this medium for about 7-21 days during which time cell division and the initiation of embryogenic callus will take place. The leaves are bombarded with chloroplast transformation vectors carrying the aadA selectable marker gene and trait gene just prior to induction of the cell proliferation genes, during induction or just after induction. One to seven days after bombardment with the chloroplast transformation vector, the tissue is placed in petri plates containing agarose-solidified media supplemented with spectinomycin. The plates are then incubated at 28° C. in the light. The tissue is transferred to fresh medium every two weeks. After about 8 weeks of incubation, green callus is observed. This tissue can be further proliferated on 13152 medium (4.3 g/l MS salts, 0.25 g/l myo-inositol, 1.0 g/l casein hydrolysate, 1 mg/l thiamine, 1 mg/l 2,4-D, 30 g/l maltose, 0.69 g/l proline, 1.2 mg/l cupric sulfate, and 3.5 g/l phytagel, pH 5.8) and the tissue analyzed for the presence of the transgene using appropriate methods including PCR and Southern analysis.

In an alternative approach, expression cassettes containing the tetracycline-inducible BBM and WUS genes are co-bombarded along with the chloroplast transformation vectors carrying the aadA gene for selection. Either leaf explants or established green tissue callus are used as the target tissue for bombardment. Tetracycline or doxycycline at a concentration of 0.5 to 2.0 mg/l is added to the culture medium (13152) after particle bombardment. Expression of BBM and WUS in cells that have received DNA stimulate callus growth rates during the period while tetracycline (or doxycycline) is present in the medium. The accelerated growth that is stimulated by BBM & WUS will result in improved recovery of homoplastic transgenic events, and the nuclear-integrated BBM/WUS genes can be removed by outcrossing TO plants to wild-type plants and selecting BBM/WUS null plants in the T1 generation.

In another variation on the particle gun approach for delivery of BBM and WUS, a UBI::BBM::PinII and a nos::WUS2::pinII are co-delivered along with the chloroplast transformation vectors.

In another alternative approach, the cell proliferation factor genes are delivered into leaf tissue by vacuum infiltration of an Agrobacterium solution. The cell proliferation factor genes are under the control of strong constitutive promoters such as ubi or act or viral promoters such as 35S (Gardner et al. (1981) Nucl Acids Res 9:2871-2888), MMV (Dey and Maiti (1999) Plant Mol Biol 40:771-782), or BSV (Shenk et al. (2001) Plant Mol Biol 47:399-412). The cell proliferation factor genes are carried on binary vectors that facilitate transfer from the bacteria to plant cells. Following vacuum infiltration, the tissue is incubated for an appropriate period of time to allow expression of the cell proliferation factor genes in the leaf tissue. Transient expression from the cell proliferation factor genes delivered by Agrobacterium is expected to provide a strong boost in cell division and tissue culture response. After vacuum infiltration with Agrobacterium, the tissue is bombarded with a chloroplast transformation vector carrying the aadA selectable marker gene. The tissue is then transferred to media containing spectinomycin and transgenic events selected. It is expected that the Agrobacterium-delivered cell proliferation factor genes will not be integrated into the nuclear genome of most of the events that are recovered.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A promoter construct comprising a promoter followed by a first attachment B (attB) site, wherein the promoter is selected from the group consisting of:
a) a promoter comprising a nucleotide sequence having the sequence set forth in SEQ ID NO: 29;
b) a promoter comprising a nucleotide sequence having at least 70% sequence identity to the sequence set forth in SEQ ID NO: 29; and
c) a promoter comprising at least 50 contiguous nucleotides of the sequence set forth in SEQ ID NO: 29; and
wherein said first attB site has the nucleotide sequence set forth in SEQ ID NO: 31.

2. The promoter construct of claim 1, wherein said first attB site modulates the activity of said promoter.

3. The promoter construct of claim 1, wherein said promoter comprises the sequence set forth in nucleotides 291-430 of SEQ ID NO: 29 or a sequence having at least 70% sequence identity to the sequence set forth in nucleotides 291-430 of SEQ ID NO: 29.

4. The promoter construct of claim 1, wherein said promoter construct further comprises a linker sequence that separates said promoter and said first attB site.

5. The promoter construct of claim 4, wherein said linker sequence that separates said promoter and said first attB site is about 133 nucleotides in length.

6. The promoter construct of claim 4, wherein said linker sequence that separates said promoter and said first attB site comprises nucleotides of a maize rab17 5′ untranslated region (5′-UTR).

7. The promoter construct of claim 6, wherein said linker sequence that separates said promoter and said first attB site comprises the nucleotide sequence set forth in SEQ ID NO: 35.

8. The promoter construct of claim 4, wherein said linker sequence that separates said promoter and said first attB site has the sequence set forth in SEQ ID NO: 36 or a nucleotide sequence having at least 70% sequence identity to the sequence set forth in SEQ ID NO: 36.

9. An expression cassette comprising the promoter construct of claim 1 operably linked to a polynucleotide of interest.

10. The expression cassette of claim 9, wherein said polynucleotide of interest is a nucleotide sequence encoding a site-specific recombinase.

11. The expression cassette of claim 10, wherein said site-specific recombinase is selected from the group consisting of FLP, Cre, SSV1, lambda Int, phi C31 Int, HK022, R, Gin, Tn1721, CinH, ParA, Tn5053, Bxb1, TP907-1, and U153.

12. The expression cassette of claim 9, further comprising a polynucleotide encoding a babyboom polypeptide operably linked to a second promoter, wherein said second promoter is active in a plant.

13. The expression cassette of claim 12, wherein said babyboom polypeptide comprises at least two AP2 domains and at least one of the following amino acid sequences:
a) the amino acid sequence set forth in SEQ ID NO. 54 or an amino acid sequence that differs from the amino acid sequence set forth in SEQ ID NO: 54 by one amino acid; and
b) the amino acid sequence set forth in SEQ ID NO: 57 or an amino acid sequence that differs from the amino acid sequence set forth in SEQ ID NO: 57 by one amino acid.

14. The expression cassette of claim 12, wherein said polynucleotide encoding said babyboom polypeptide has a nucleotide sequence selected from the group consisting of:
a) the nucleotide sequence set forth in SEQ ID NO: 1, 3, 5, 7, 9, 11, 13, 15, 17, 19, 21, 23, 25, 27, 68, 116, 117, 120, 121, or 69;
b) a nucleotide sequence having at least 70% sequence identity to SEQ ID NO: 1, 3, 5, 7, 9, 11, 13, 15, 17, 19, 21, 23, 25, 27, 68, 116, 117, 120, 121, or 69;
c) a nucleotide sequence encoding a polypeptide having the amino acid sequence set forth in SEQ ID NO: 2, 4, 6, 8, 10, 12, 14, 16, 18, 20, 22, 24, 26, 122, or 28; and
d) a nucleotide sequence encoding a polypeptide having an amino acid sequence having at least 70% sequence identity to the amino acid sequence set forth in SEQ ID NO: 2, 4, 6, 8, 10, 12, 14, 16, 18, 20, 22, 24, 26, 122, or 28.

15. The expression cassette of claim 12, wherein said second promoter is a maize ubiquitin promoter or a maize oleosin promoter.

16. The expression cassette of claim 12, wherein said expression cassette further comprises a polynucleotide encoding a Wuschel polypeptide operably linked to a third promoter, wherein said third promoter is active in a plant.

17. The expression cassette of claim 16, wherein said polynucleotide encoding said Wuschel polypeptide has a nucleotide sequence selected from the group consisting of:
a) the nucleotide sequence set forth in SEQ ID NO: 61, 63, 114, or 105; and
b) a nucleotide sequence having at least 70% sequence identity to SEQ ID NO: 61, 63, 114, or 105;
c) a nucleotide sequence encoding a polypeptide having the amino acid sequence set forth in SEQ ID NO: 62, 64, 115, or 106; and
d) a nucleotide sequence encoding a polypeptide having an amino acid sequence having at least 70% sequence identity to SEQ ID NO: 62, 64, 115, or 106.

18. The expression cassette of claim 16, wherein said third promoter is a maize In2-2 promoter or a nopaline synthase promoter.

19. A host cell comprising the expression cassette of claim 9.

20. A plant cell comprising the expression cassette of claim 9.

21. A transgenic seed of a plant comprising the plant cell of claim 20, wherein said seed comprises said expression cassette.

22. A plant cell comprising an expression cassette comprising a promoter construct according to claim 1 operably linked to a polynucleotide encoding a site-specific recombinase, wherein said plant cell further comprises a polynucleotide of interest flanked by a first and a second recombination site, wherein said first and second recombination sites are recombinogenic with respect to one another and are directly repeated, and wherein said site-specific recombinase can recognize and implement recombination at said first and said second recombination sites, thereby excising said polynucleotide of interest.

23. A method for expressing a polynucleotide of interest in a plant cell, said method comprising introducing into said plant cell an expression cassette, wherein said expression cassette is the expression cassette according to claim 9.

24. A method for excising a polynucleotide of interest from a target site in a plant cell, wherein said target site comprises a first site-specific recombination site, said polynucleotide of interest, and a second site-specific recombination site, wherein said first and said second site-specific recombination sites are recombinogenic with respect to one another and are directly repeated, said method comprising:
a) introducing into said plant cell an expression cassette comprising a first promoter operably linked to a polynucleotide encoding a site-specific recombinase, wherein said first promoter comprises the promoter construct of claim 1; and
b) expressing said polynucleotide encoding said site-specific recombinase, wherein said site-specific recombinase recognizes and implements recombination at said first and said second site-specific recombination sites, thereby excising said polynucleotide of interest.

25. The method of claim 24, wherein said target site comprises in operable linkage: said first recombination site, a second promoter, a polynucleotide sequence encoding a Wuschel polypeptide, a third promoter, a polynucleotide encoding a babyboom polypeptide, and said second recombination site, wherein said polynucleotide encoding said Wuschel polypeptide and its operably linked second promoter can follow or precede said polynucleotide encoding said babyboom polypeptide and its operably linked third promoter in the target site.

26. A method for excising a polynucleotide of interest from a target site in a plant cell, wherein said target site comprises in operable linkage: a first site-specific recombination site, a first promoter, said polynucleotide of interest, a second promoter, a polynucleotide encoding a site-specific recombinase, and a second site-specific recombination site; wherein said first and said second site-specific recombination sites are recombinogenic with respect to one another and are directly repeated: wherein said second promoter comprises the promoter construct of claim 1, wherein the polynucleotide of interest and its operably linked first promoter can follow or precede the polynucleotide encoding the site-specific recombinase and its operably linked second promoter in the target site; said method comprising expressing said polynucleotide encoding said site-specific recombinase; wherein said site-specific recombinase recognizes and implements recombination at said first and said second site-specific recombination sites, thereby excising said polynucleotide of interest and said polynucleotide encoding said site-specific recombinase.

27. The method of claim 26, wherein said first promoter is a maize ubiquitin promoter or a maize oleosin promoter.

28. The method of claim 26, wherein said target site further comprises a third promoter operably linked to a polynucleotide encoding a Wuschel polypeptide, wherein the target site comprises in operable linkage: said first site-specific recombination site, said third promoter, said polynucleotide encoding a Wuschel polypeptide, said first promoter, said polynucleotide of interest, said second promoter, said polynucleotide encoding said site-specific recombinase, and said second site-specific recombination site, wherein the polynucleotide encoding the Wuschel polypeptide and its operably linked third promoter, the polynucleotide of interest and its operably linked first promoter, and the polynucleotide encoding the site-specific recombinase and its operably linked second promoter can be in any order at the target site.

29. The method of claim 28, wherein the target site comprises in the following order: said first site-specific recombination site, said third promoter, said polynucleotide encoding a Wuschel polypeptide, said first promoter, said polynucleotide of interest, said second promoter, said polynucleotide encoding said site-specific recombinase, and said second site-specific recombination site.

30. The method of claim 26, wherein said site-specific recombinase is selected from the group consisting of FLP, Cre, SSV1, lambda Int, phi C31 Int, HK022, R, Gin, Tn1721, CinH, ParA, Tn5053, Bxb1, TP907-1, and U153.

31. The method of claim 26, wherein said polynucleotide of interest is a polynucleotide encoding a cell proliferation factor.

32. The method of claim 31, wherein said cell proliferation factor comprises a babyboom polypeptide.

33. The method of claim 32, wherein said babyboom polypeptide comprises at least two AP2 domains and at least one of the following amino acid sequences:
a) the amino acid sequence set forth in SEQ ID NO: 54 or an amino acid sequence that differs from the amino acid sequence set forth in SEQ ID NO: 54 by one amino acid; and
b) the amino acid sequence set forth in SEQ ID NO: 57 or an amino acid sequence that differs from the amino acid sequence set forth in SEQ ID NO: 57 by one amino acid.

34. The method of claim 32, wherein said polynucleotide encoding said babyboom polypeptide has a nucleotide sequence selected from the group consisting of:
a) the nucleotide sequence set forth in SEQ ID NO: 1, 3, 5, 7, 9, 11, 13, 15, 17, 19, 21, 23, 25, 27, 68, 116, 117, 120, 121, or 69;
b) a nucleotide sequence having at least 70% sequence identity to SEQ ID NO: 1, 3, 5, 7, 9, 11, 13, 15, 17, 19, 21, 23, 25, 27, 68, 116, 117, 120, 121, or 69;
c) a nucleotide sequence encoding a polypeptide having the amino acid sequence set forth in SEQ ID NO: 2, 4, 6, 8, 10, 12, 14, 16, 18, 20, 22, 24, 26, 122, or 28; and
d) a nucleotide sequence encoding a polypeptide having an amino acid sequence having at least 70% sequence identity to the amino acid sequence set forth in SEQ ID NO: 2, 4, 6, 8, 10, 12, 14, 16, 18, 20, 22, 24, 26, 122, or 28.

35. A method for transforming a plastid of a plant cell with a polynucleotide of interest, said method comprising:
a) introducing into a plant cell a heterologous polynucleotide encoding a cell proliferation factor;
b) expressing said heterologous polynucleotide encoding said cell proliferation factor; and
c) introducing into a plastid of said plant cell a vector comprising said polynucleotide of interest, wherein the heterologous polynucleotide encoding said cell proliferation factor is expressed prior to, during, or following the introduction into said plastid of said vector comprising said polynucleotide of interest.

36. The method of claim 35, wherein said cell proliferation factor is a babyboom polypeptide.

37. The method of claim 35, wherein said heterologous polynucleotide encoding said cell proliferation factor is operably linked to a promoter, and wherein said promoter is part of a tetracycline-repressor system, and expressing said heterologous polynucleotide comprises providing to said plant cell doxycyline.

38. The method of claim 35, wherein said plastid is a chloroplast, amyloplast, or chromoplast.

39. A method for introducing a polynucleotide of interest into a mature embryo explant of a mature seed, said method comprising:
a) dissecting a mature embryo from a mature seed;
b) making slices of said mature embryo to prepare said mature embryo explant, wherein said mature embryo explant comprises at least one of the tissues selected from the group consisting of leaf primordia, mesocotyl, shoot apical meristem, and root primordia; and
c) introducing into said mature embryo explant:
i) a heterologous polynucleotide encoding a cell proliferation factor and expressing said heterologous polynucleotide encoding said cell proliferation factor; and
ii) a polynucleotide of interest.

40. The method of claim 39, wherein said mature embryo explant comprises leaf primordia, mesocotyl, and root primordia.

41. The method of claim 39, wherein said cell proliferation factor comprises a babyboom polypeptide.

42. A method for introducing a polynucleotide of interest into a leaf tissue and regenerating a plant therefrom, said method comprising:
a) excising a leaf segment from a leaf above the first leaf base node;
b) dissecting said leaf fragment into leaf tissue;
d) introducing into said leaf tissue:
i) a heterologous polynucleotide encoding a cell proliferation factor flanked by recombination sites;
ii) an expression cassette comprising the promoter construct according to claim 1 operably linked to a polynucleotide encoding a site-specific recombinase that is capable of recognizing and implementing recombination at said recombination sites; and
iii) a polynucleotide of interest;
e) expressing said heterologous polynucleotide encoding said cell proliferation factor;
f) incubating said leaf tissue under conditions to allow for growth of a callus;
g) expressing said polynucleotide encoding said site-specific recombinase, thereby excising said heterologous polynucleotide encoding said cell proliferation factor;
h) regenerating a plant from said callus.

43. The method of claim 42, wherein said cell proliferation factor comprises a babyboom polypeptide.

44. An isolated polypeptide comprising an amino acid sequence selected from the group consisting of:
a) the amino acid sequence set forth in SEQ ID NO: 2, 4, 8, or 18; and
b) an amino acid sequence having at least 75% sequence identity to the sequence set forth in SEQ ID NO: 2, wherein said polypeptide has BBM activity;
c) an amino acid sequence having at least 75% sequence identity to the sequence set forth in SEQ ID NO: 4, wherein said amino acid sequence has amino acid residues valine, tyrosine, and leucine at the positions corresponding to positions 311, 312, and 313, respectively of SEQ ID NO: 4, wherein said polypeptide has BBM activity;
d) an amino acid sequence having at least 75% sequence identity to the sequence set forth in SEQ ID NO; 8, wherein said amino acid sequence has amino acid residues methionine, alanine, and serine at the positions corresponding to positions 1, 2, and 3, respectively, of SEQ ID NO: 8, wherein said polypeptide has BBM activity; and
e) an amino acid sequence having at least 75% sequence identity to the sequence set forth in SEQ ID NO 18, wherein said amino acid sequence has amino acid residues valine, tyrosine, and leucine at the positions corresponding to positions 337, 338, and 339, respectively, of SEQ ID NO. 18, wherein said polypeptide has 13M activity.

45. An isolated polynucleotide comprising a nucleotide sequence selected from the group consisting of:
a) a nucleotide sequence comprising the sequence set forth in SEQ ID NO: 1, 3, 7, or 17;
b) a nucleotide sequence having at least 75% sequence identity to the sequence set forth in SEQ ID NO: 1, wherein said nucleotide sequence encodes a polypeptide having BBM activity;
c) a nucleotide sequence encoding the amino acid sequence set forth in SEQ ID NO: 2, 4, 8, or 18; and
d) a nucleotide sequence encoding a polypeptide having an amino acid sequence having at least 75% sequence identity to the amino acid sequence set forth in SEQ ID NO: 2, wherein said polypeptide has BBM activity;
e) a nucleotide sequence encoding a polypeptide having an amino acid sequence having at least 75% sequence identity to the sequence set forth in SEQ ID NO: 4, wherein said amino acid sequence has amino acid residues valine, tyrosine, and leucine at the positions corresponding to positions 311, 312, and 313, respectively of SEQ ID NO: 4, wherein said polypeptide has B3M activity;
f) a nucleotide sequence encoding a polypeptide having an amino acid sequence having at least 75% sequence identity to the sequence set forth in SEQ ID NO; 8, wherein said amino acid sequence has amino acid residues methionine, alanine, and serine at the positions corresponding to positions 1, 2, and 3, respectively, of SEQ ID NO: 8, wherein said polypeptide has BBM activity; and
g) a nucleotide sequence encoding a polypeptide having an amino acid sequence having at least 75% sequence identity to the sequence set forth in SEQ ID NO: 18, wherein said amino acid sequence has amino acid residues valine, tyrosine, and leucine at the positions corresponding to positions 337, 338, and 339, respectively, of SEQ ID NO: 18, wherein said polypeptide has BBM activity;
h) the complement of any one of the nucleotide sequences of a), b), c), d), e), f), or g).

46. An expression cassette comprising the isolated polynucleotide of claim 45, wherein said polynucleotide is operably linked to a promoter that drives expression in a plant.

47. A plant comprising a heterologous polynucleotide operably linked to a promoter that drives expression in the plant, wherein said polynucleotide is selected from the group consisting of:
a) a polynucleotide comprising the nucleotide sequence set forth in SEQ ID NO: 1, 3, 5, 7, 17, or 27; or a complement thereof;
b) a polynucleotide comprising a nucleotide sequence having at least 75% sequence identity to the sequence set forth in SEQ ID NO: 1, 3, 5, 17, or 27; wherein said nucleotide sequence encodes a polypeptide having BBM activity;
c) a polynucleotide encoding the amino acid sequence set forth in SEQ ID NO: 2, 4, 6, 8, 18, or 28;
d) a polynucleotide encoding an amino acid sequence having at least 75% sequence identity to the sequence set forth in SEQ ID NO; 8, wherein said amino acid sequence has amino acid residues methionine, alanine, and serine at the positions corresponding to positions 1, 2, and 3, respectively, of SEQ ID NO: 8, wherein said polynucleotide encodes a polypeptide having BBM activity; and
e) a polynucleotide encoding an amino acid sequence having at least 75% sequence identity to the amino acid sequence set forth in SEQ ID NO: 2, 4, 6, 18, or 28; wherein said polynucleotide encodes a polypeptide having BBM activity.

48. A method of increasing the activity of a polypeptide in a plant comprising providing to said plant a polypeptide selected from the group consisting of:
a) the polypeptide comprising the amino acid sequence of SEQ ID NO: 2, 4, 6, 8, 18, or 28;
b) the polypeptide having at least 75% sequence identity to SEQ ID NO: 2, 4, 6, 18, or 28, wherein said polypeptide has BBM activity; and
c) the polypeptide having an amino acid sequence having at least 75% sequence identity to the sequence set forth in SEQ ID NO; 8, wherein said amino acid sequence has amino acid residues methionine, alanine, and serine at the positions corresponding to positions 1, 2, and 3, respectively, of SEQ ID NO: 8, wherein said polypeptide has BBM activity; and
wherein increasing the activity of said polypeptide produces a phenotype in the plant selected from the group consisting of:
a) produces asexually derived embryos in the plant;
b) modifies the regenerative capacity of the plant;
c) increases the transformation efficiency in the plant;
d) increases or maintains the yield in the plant under abiotic stress; and
e) induces embryogenesis.

49. The method of claim 48, wherein providing the polypeptide comprises introducing into said plant a heterologous polynucleotide selected from the group consisting of:
a) the polynucleotide comprising SEQ ID NO: 1, 3, 5, 7, 17, or 27;
b) the polynucleotide encoding the amino acid sequence of SEQ ID NO: 2, 4, 6, 8, 18, or 28;
c) the polynucleotide having at least 75% sequence identity to SEQ ID NO: 1, 3, 5, 17, or 27, wherein said polynucleotide encodes a polypeptide having BBM activity;
d) the polynucleotide encoding an amino acid sequence having at least 75% sequence identity to SEQ ID NO: 2, 4, 6, 18, or 28, wherein said polynucleotide encodes a polypeptide having BBM activity; and
e) the polynucleotide encoding an amino acid sequence having at least 75% sequence identity to the sequence set forth in SEQ ID NO; 8, wherein said amino acid sequence has amino acid residues methionine, alanine, and serine at the positions corresponding to positions 1, 2, and 3, respectively, of SEQ ID NO: 8, wherein said polynucleotide encodes a polypeptide having BBM activity.

50. A method of transforming a plant cell comprising:
(a) transforming a plant cell with a polynucleotide of interest and a heterologous polynucleotide comprising a first nucleotide sequence selected from the group consisting of:
a) a nucleotide sequence comprising SEQ ID NO: 1, 3, 5, 7, 17, or 27;
b) a nucleotide sequence encoding the amino acid sequence of SEQ ID NO: 2, 4, 6, 8, 18, or 28;
c) a nucleotide sequence having at least 75% sequence identity to SEQ ID NO: 1, 3, 5, 17, or 27, wherein said nucleotide sequence encodes a polypeptide having BBM activity;
d) a nucleotide sequence encoding an amino acid sequence having at least 75% sequence identity to SEQ ID NO: 2, 4, 6, 18, or 28, wherein said nucleotide sequence encodes a polypeptide having BBM activity; and
e) a nucleotide sequence encoding an amino acid sequence having at least 75% sequence identity to the sequence set forth in SEQ ID NO; 8, wherein said amino acid sequence has amino acid residues methionine, alanine, and serine at the positions corresponding to positions 1, 2, and 3, respectively, of SEQ ID NO: 8, wherein said nucleotide sequence encodes a polypeptide having BBM activity.


SEQUENCE LISTING (126 sequences)

SEQ ID NO: 1 - DNA, 2133 bp, Glycine max
  source 1..2133: mol_type = genomic DNA
  CDS 1..2133
atggggtctatgaatttgttaggtttttctctctctcctcaagaacacccttctagtcaagatcactctcaaacggcaccttctcgtttttgcttcaaccctgatggaatctcaagcactgatgtagcaggagactgctttgatctcacttctgactcaactcctcatttactcaaccttccctcttacggcatatacgaagcttttcataggagcaacaatattcacaccactcaagattggaaggagaactacaacagccaaaacttgctattgggaacttcatgcagcaaccaaaacatgaaccacaaccatcagcaacaacaacaacaacagccaaagcttgaaaacttcctcggtggacactcatttggtgaacatgagcaaccctacggtggtaactcagcctctacagaatacatgttcccggctcagccggtattggccggtggcggcggcggtggtagcaatagcagcaacacaagcaacagtagctccatagggttatccatgataaagacatggttgaggaaccaaccaccacactcagaaaacaacaataacaacaacaatgaaagtggtggcaatagtagaagcagtgtgcagcagactctatcactttccatgagtactggttcacaatcaagcacatcactaccccttctcactgctagtgtggataatggagagagttcttctgataacaaacaaccacataccacggctgcacttgatacaacccaaaccggagccattgaaactgcacccagaaagtccattgacacttttggacagagaacttctatctaccgtggtgtaacaaggcataggtggacggggaggtatgaggctcacctgtgggataatagttgtagaagagagggacaaactcgcaaaggaaggcaagtttacttgggaggttatgacaaagaagaaaaggcagctagagcctacgatttggcagcactaaaatactggggaacaactacgacaacaaattttccaattagccactatgagaaagagttggaagaaatgaagcacatgactaggcaagagtacgttgcgtcattgagaaggaagagtagtgggttttctcgcggggcatccatttatcgaggtgtgacgagacaccatcaacatggaagatggcaagcgaggattggaagagttgctggcaacaaggatctctacttgggaactttcagcacccaagaggaggcagcagaagcatatgatgtagcagcaatcaaattcagaggactaagtgctgttacaaactttgacatgagcagatatgacgtgaaaagcatacttgagagcaccactttgccaattggtggtgctgcaaagcgtttgaaggatatggagcaggtggaactgagggtggagaatgttcatagagcagatcaagaagatcatagtagcatcatgaactctcacttaactcaaggaatcattaacaactatgcagcaggaggaacaacagcgactcatcatcataactggcacaatgctcttgcattccaccaacctcaaccttgcaccaccatacactacccttatggacaaagaattaattggtgcaagcaagaacaagacaactctgatgcctctcactctttgtcttattcagatattcatcaactacagctagggaacaatggcacacacaacttctttcacacaaattcagggttgcaccctatgttaagcatggattctgcttccattgacaatagctcttcatctaactctgttgtttatgatggttatggaggtggtgggggctataatgtgattcctatggggactactactactgttgttgcaaatgatggtgatcaaaatccaagaagcaatcatggttttggtgataatgagataaaggcacttggttatgaaagtgtgtatggttctacaactgatccttatcatgcacatgcaaggaacttgtattatcttactcaacagcaaccatcttctgttgatgcagtgaaggctagtgcatatgatcaaggatctgcatgcaatacttgggttccaactgctattccaactcatgcaccaaggtctagtactagtatggctctctgccatggtgctacgcccttctctttattgcatgaatag

SEQ ID NO: 2 - protein, 710 aa, Glycine max
MGSMNLLGFSLSPQEHPSSQDHSQTAPSRFCFNPDGISSTDVAGDCFDLTSDSTPHLLNLPSYGIYEAFHRSNNIHTTQDWKENYNSQNLLLGTSCSNQNMNHNHQQQQQQQPKLENFLGGHSFGEHEQPYGGNSASTEYMFPAQPVLAGGGGGGSNSSNTSNSSSIGLSMIKTWLRNQPPHSENNNNNNNESGGNSRSSVQQTLSLSMSTGSQSSTSLPLLTASVDNGESSSDNKQPHTTAALDTTQTGAIETAPRKSIDTFGQRTSIYRGVTRHRWTGRYEAHLWDNSCRREGQTRKGRQVYLGGYDKEEKAARAYDLAALKYWGTTTTTNFPISHYEKELEEMKHMTRQEYVASLRRKSSGFSRGASIYRGVTRHHQHGRWQARIGRVAGNKDLYLGTFSTQEEAAEAYDVAAIKFRGLSAVTNFDMSRYDVKSILESTTLPIGGAAKRLKDMEQVELRVENVHRADQEDHSSIMNSHLTQGIINNYAAGGTTATHHHNWHNALAFHQPQPCTTIHYPYGQRINWCKQEQDNSDASHSLSYSDIHQLQLGNNGTHNFFHTNSGLHPMLSMDSASIDNSSSSNSVVYDGYGGGGGYNVIPMGTTTTVVANDGDQNPRSNHGFGDNEIKALGYESVYGSTTDPYHAHARNLYYLTQQQPSSVDAVKASAYDQGSACNTWVPTAIPTHAPRSSTSMALCHGATPFSLLHE

SEQ ID NO: 3 - DNA, 2112 bp, Sorghum bicolor
  source 1..2112: mol_type = genomic DNA
  CDS 1..2112
atggctactgtgaacaactggctcgctttctccctctccccgcaggagctgccgcccacccagacggactccaccctcatctctgccgccaccaccgacgatgtctccggcgatgtctgcttcaacatcccccaagattggagcatgaggggatccgagctttcggcgctcgtcgccgagccgaagctggaggacttcctcggcggaatctccttctccgagcagcaccacaaggccaactgcaacatgatccccagcactagcagcacagcttgctacgcgagctcgggtgctaccgccggctaccatcaccagctgtaccaccagcccaccagctccgcgctccacttcgctgactccgtcatggtggcctcctcggccggcggcgtccacgacggaggtgccatgctcagcgcggccagcgctaatggtagcgctggcgctggcgctgccagtgccaatggcagcggcagcatcgggctgtccatgatcaagaactggctgcggagccaaccagctcccatgcagccgagggtggcggcggctgagagcgtgcaggggctctctttgtccatgaacatggcgggggcgacgcaaggcgccgctggcatgccacttcttgctggagagcgcggccgggcgcccgagagtgtctcgacgtcggcacagggtggagccgtcgtcacggctccaaaggaggatagcggtggcagcggtgttgccgccaccggcgccctagtagccgtgagcacggacacgggtggcagcggcgcgtcggctgacaacacggcaaggaagacggtggacacgttcgggcagcgcacgtcgatttaccgtggcgtgacaaggcatagatggactgggagatatgaagcacatctgtgggacaacagttgcagaagggaaggacaaactcgcaagggtcgtcaagtctatttaggtggctatgataaagaggagaaagctgctagggcttatgatctggctgctcttaagtactggggtcccacgacaacaacaaattttccagtgaataactacgaaaaggagctggaggatatgaagcacatgacaaggcaggagtttgtagcgtctctgagaaggaagagcagtggtttctccagaggtgcatccatttacaggggagtgactaggcatcaccagcatggaagatggcaagcacggattggacgagttgcagggaacaaggatctctacttgggcaccttcagcacgcaggaggaggcagcggaggcatacgacattgcggcgatcaagttccgcggcctcaacgccgtcacaaacttcgacatgagccgctacgacgtcaagagcatcctggacagcagtgcgctccccatcggcagcgccgccaagcgtctcaaggaggccgaggccgccgcgtccgcacagcaccatgccggcgtggtgagctacgacgtcggccgcatagcctcacagctcggcgacggcggcgccctggcggcggcgtacggcgcgcactaccatggcgcctggccgaccatcgcgttccagccgagcgcggccacgggcctgtaccacccgtacgcgcagccgatgcgcgggtggtgcaagcaggagcaggaccacgcggtgatcgcggccgcgcacagcctgcaggagctccaccacctgaacctgggtgctgccgccggcgcgcacgacttcttctcggcggggcagcaggcggcgatgcacggcctgggtagcatggacaatgcatcactcgagcacagcaccggctccaactccgtcgtgtacaacggtgttggtgatagcaacggcagcaccgtcgtcggcagtggtggctacatgatgcctatgagcgctgccacggcgacggctaccacggcaatggtgagccacgagcaggtgcatgcacgggcacagggtgatcaccacgacgaagccaagcaggctgctcagatggggtacgagagctacctggtgaacgcagagaactatggcggcgggaggatgtctgcggcctgggcgactgtctcagcgccaccggcggcaagcagcaacgataacatggcggacgtcggccatggcggcgcacagctcttcagtgtctggaacgatacttaa

SEQ ID NO: 4 - protein, 703 aa, Sorghum bicolor
MATVNNWLAFSLSPQELPPTQTDSTLISAATTDDVSGDVCFNIPQDWSMRGSELSALVAEPKLEDFLGGISFSEQHHKANCNMIPSTSSTACYASSGATAGYHHQLYHQPTSSALHFADSVMVASSAGGVHDGGAMLSAASANGSAGAGAASANGSGSIGLSMIKNWLRSQPAPMQPRVAAAESVQGLSLSMNMAGATQGAAGMPLLAGERGRAPESVSTSAQGGAVVTAPKEDSGGSGVAATGALVAVSTDTGGSGASADNTARKTVDTFGQRTSIYRGVTRHRWTGRYEAHLWDNSCRREGQTRKGRQVYLGGYDKEEKAARAYDLAALKYWGPTTTTNFPVNNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHAGVVSYDVGRIASQLGDGGALAAAYGAHYHGAWPTIAFQPSAATGLYHPYAQPMRGWCKQEQDHAVIAAAHSLQELHHLNLGAAAGAHDFFSAGQQAAMHGLGSMDNASLEHSTGSNSVVYNGVGDSNGSTVVGSGGYMMPMSAATATATTAMVSHEQVHARAQGDHHDEAKQAAQMGYESYLVNAENYGGGRMSAAWATVSAPPAASSNDNMADVGHGGAQLFSVWNDT

SEQ ID NO: 5 - DNA, 1932 bp, Vitis vinifera
  source 1..1932: mol_type = genomic DNA
  CDS 1..1932
atggcttccatgaacaactggttgggtttctctttgtcccctcgagaacttccaccacagcctgaaaatcactcacagaacagtgtctctagacttggtttcaactctgatgaaatctctgggactgatgtgtcaggtgagtgttttgatctcacttcagattccactgctccctctctcaacctccctcccccttttgggatacttgaagcattcaacaggaataatcagccccaagatactaactacaaaaccaccacttctgagctctccatgctcatgggtagttcatgcagtagtcatcataacctcgaaaaccaagaacccaaacttgaaaatttcctgggctgccgctcttttgctgatcatgagcagaaacttcaagggtactacatttccattggtttatccatgatcaagacatggctgcggaaccaacctgcacccacccatcaggataacaacaagagtactgatactgggcctgtcggtggagccgccgctgggaacctacccaatgcacagaccttatcgttgtccatgagcaccggctcgcaccagaccggtgccattgaaacggtgccaaggaagtccattgatacatttggacagaggacatccatataccgtggtgtaacaaggcatagatggacgggtagatatgaggctcatctatgggacaacagttgcagaagagaaggacaaactcgaaagggaaggcaagtttatttaggtggttatgacaaagaagaaaaggcagctagggcttacgatttagcagcactgaagtattggggtaccaccaccacaacaaatttccctattagcaactatgaaaaagagatagaggagatgaagcacatgacaaggcaggagtacgtagcatctctgcgaaggaagagtagcgggttttctcgtggagcatccatatatagaggagtgaccagacaccatcagcatgggagatggcaggcaaggattggaagagtcgcaggcaacaaagatctttacttgggaactttcagcacccaagaggaagcagcagaggcctatgacattgctgccattaagtttcgaggattgaatgcggtgaccaactttgatatgagtagatatgatgttaatagcattctagagagcagtaccttgccgattggtggagctgcaaagcggttgaaagatgctgagcaggctgaaatgactatagatggacagaggacagacgatgagatgagctcacagctgactgatggaatcaacaactatggagcacaccaccatggctggcctactgttgcattccaacaagctcagccatttagcatgcactacccttatggccatcagcagagggctgtttggtgtaagcaagagcaagaccctgatggcacacacaactttcaagatcttcaccaactacaattgggaaacactcacaacttcttccagcctaatgttctgcacaacctcatgagcatggactcttcttcaatggaccatagctcaggctccaattcagtcatctatagcggtggtggagccgctgatggcagcgctgcaactggcggcagtggcagtgggagcttccaaggggtaggttatgggaacaacattggctttgtgatgcccataagcaccgtcatcgctcatgaaggcggccatggccagggaaatggtggctttggagatagcgaagtgaaggcgattggttacgacaacatgtttggatcgacagatccttaccatgctaggagcttgtactatctttcacagcaatcatctgcaggcatggtgaagggcagtagtgcatatgatcaggggtcagggtgtaacaactgggttccaactgcagttccaaccctagctccaaggactaacagcttggcagtatgccatggaacacctacattcacagtatggaatgatacataa

SEQ ID NO: 6 - protein, 643 aa, Vitis vinifera
MASMNNWLGFSLSPRELPPQPENHSQNSVSRLGFNSDEISGTDVSGECFDLTSDSTAPSLNLPPPFGILEAFNRNNQPQDTNYKTTTSELSMLMGSSCSSHHNLENQEPKLENFLGCRSFADHEQKLQGYYISIGLSMIKTWLRNQPAPTHQDNNKSTDTGPVGGAAAGNLPNAQTLSLSMSTGSHQTGAIETVPRKSIDTFGQRTSIYRGVTRHRWTGRYEAHLWDNSCRREGQTRKGRQVYLGGYDKEEKAARAYDLAALKYWGTTTTTNFPISNYEKEIEEMKHMTRQEYVASLRRKSSGFSRGASIYRGVTRHHQHGRWQARIGRVAGNKDLYLGTFSTQEEAAEAYDIAAIKFRGLNAVTNFDMSRYDVNSILESSTLPIGGAAKRLKDAEQAEMTIDGQRTDDEMSSQLTDGINNYGAHHHGWPTVAFQQAQPFSMHYPYGHQQRAVWCKQEQDPDGTHNFQDLHQLQLGNTHNFFQPNVLHNLMSMDSSSMDHSSGSNSVIYSGGGAADGSAATGGSGSGSFQGVGYGNNIGFVMPISTVIAHEGGHGQGNGGFGDSEVKAIGYDNMFGSTDPYHARSLYYLSQQSSAGMVKGSSAYDQGSGCNNWVPTAVPTLAPRTNSLAVCHGTPTFTVWNDT

SEQ ID NO: 7 - DNA, 2070 bp, Medicago truncatula
  source 1..2070: mol_type = genomic DNA
  CDS 1..2070
atggcctctatgaacttgttaggtttctctctatctccacaagaacaacatccatcaacacaagatcaaacggtggcttcccgttttgggttcaaccctaatgaaatctcaggctctgatgttcaaggagatcactgctatgatctctcttctcacacaactcctcatcattcactcaacctttctcatcctttttccatttatgaagctttccacacaaataacaacattcacaccactcaagattggaaggagaactacaacaaccaaaacctactattgggaacatcatgcatgaaccaaaatgtgaacaacaacaaccaacaagcacaaccaaagctagaaaacttcctcggtggacactctttcaccgaccatcaagaatacggtggtagcaactcatactcttcattacacctcccacctcatcagccggaagcatcctgtggcggtggtgatggtagtacaagtaacaataactcaataggtttatctatgataaaaacatggctcagaaaccaaccaccaccaccagaaaacaacaacaataacaacaatgaaagtggtgcacgtgtgcagacactatcactttctatgagtactggctcacagtcaagttcatctgtgcctcttctcaatgcaaatgtgatgagtggtgagatttcctcatcggaaaacaaacaaccacccacaactgcagttgtacttgatagcaaccaaacaagtgtcgttgaaagtgctgtgcctagaaaatccgttgatacatttggacaaagaacttccatttaccgtggtgtaacaaggcatagatggacagggagatatgaagctcacctttgggataatagttgtagaagagaggggcagactcgcaaaggaaggcaagtttacttgggaggttatgacaaagaagaaaaagcagctagagcctatgatttggcagcactaaaatattggggaacaactactacaacaaattttccaattagccattatgaaaaagaagtggaagaaatgaagcatatgacaaggcaagagtacgttgcgtcattgagaaggaaaagtagtggtttttcacgaggtgcatccatttaccgaggagtaacaagacatcatcaacatggtagatggcaagctaggattggaagagttgcaggcaacaaagatctctacctaggaactttcagcactcaagaagaggcagcagaggcatatgatgtggcagcaataaaattcagaggactgagtgcagttacaaactttgacatgagcagatatgatgtcaaaaccatacttgagagcagcacattaccaattggtggtgctgcaaagcgtttaaaagacatggagcaagttgaattgaatcatgtgaatgttgatattagccatagaactgaacaagatcatagcatcatcaacaacacttcccatttaacagaacaagccatctatgcagcaacaaatgcatctaattggcatgcactttcattccaacatcaacaaccacatcatcattacaatgccaacaacatgcagttacagaattatccttatggaactcaaactcaaaagctttggtgcaaacaagaacaagattctgatgatcatagtacttatactactgctactgatattcatcaactacagttagggaataataataacaatactcacaatttctttggtttacaaaatatcatgagtatggattctgcttccatggataatagttctggatctaattctgttgtttatggtggtggagatcatggtggttatggaggaaatggtggatatatgattccaatggctattgcaaatgatggtaaccaaaatccaagaagcaacaacaattttggtgagagtgagattaaaggatttggttatgaaaatgtttttgggactactactgatccttatcatgcacaggcagcaaggaacttgtactatcagccacaacaattatctgttgatcaaggatcaaattgggttccaactgctattccaacacttgctccaaggactaccaatgtctctctatgtcctcctttcactttgttgcatgaatag

SEQ ID NO: 8 - protein, 689 aa, Medicago truncatula
MASMNLLGFSLSPQEQHPSTQDQTVASRFGFNPNEISGSDVQGDHCYDLSSHTTPHHSLNLSHPFSIYEAFHTNNNIHTTQDWKENYNNQNLLLGTSCMNQNVNNNNQQAQPKLENFLGGHSFTDHQEYGGSNSYSSLHLPPHQPEASCGGGDGSTSNNNSIGLSMIKTWLRNQPPPPENNNNNNNESGARVQTLSLSMSTGSQSSSSVPLLNANVMSGEISSSENKQPPTTAVVLDSNQTSVVESAVPRKSVDTFGQRTSIYRGVTRHRWTGRYEAHLWDNSCRREGQTRKGRQVYLGGYDKEEKAARAYDLAALKYWGTTTTTNFPISHYEKEVEEMKHMTRQEYVASLRRKSSGFSRGASIYRGVTRHHQHGRWQARIGRVAGNKDLYLGTFSTQEEAAEAYDVAAIKFRGLSAVTNFDMSRYDVKTILESSTLPIGGAAKRLKDMEQVELNHVNVDISHRTEQDHSIINNTSHLTEQAIYAATNASNWHALSFQHQQPHHHYNANNMQLQNYPYGTQTQKLWCKQEQDSDDHSTYTTATDIHQLQLGNNNNNTHNFFGLQNIMSMDSASMDNSSGSNSVVYGGGDHGGYGGNGGYMIPMAIANDGNQNPRSNNNFGESEIKGFGYENVFGTTTDPYHAQAARNLYYQPQQLSVDQGSNWVPTAIPTLAPRTTNVSLCPPFTLLHE

SEQ ID NO: 9 - DNA, 2130 bp, Zea mays
  source 1..2130: mol_type = genomic DNA
  CDS 1..2130
atggccactgtgaacaactggctcgctttctccctctccccgcaggagctgccgccctcccagacgacggactccacgctcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaagtccaactgcaacttgatacccagcactagcagcacagtttgctacgcgagctcagctgctagcaccggctaccatcaccagctgtaccagcccaccagctccgcgctccacttcgcggactccgtcatggtggcctcctcggccggtgtccacgacggcggttccatgctcagcgcggccgccgctaacggtgtcgctggcgctgccagtgccaacggcggcggcatcgggctgtccatgatcaagaactggctgcggagccaaccggcgcccatgcagccgagggcggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggtgccgtcgtcgtcacggcgccgaaggaggatagcggtggcagcggtgttgccggtgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaaggacaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgtctcaaggaggccgaggccgcagcgtccgcgcagcaccaccacgccggcgtggtgagctacgacgtcggccgcatcgcctcgcagctcggcgacggcggagccctagcggcggcgtacggcgcgcactaccacggcgccgcctggccgaccatcgcgttccagccgggcgccgccaccacaggcctgtaccacccgtacgcgcagcagccaatgcgcggcggcgggtggtgcaagcaggagcaggaccacgcggtgatcgcggccgcgcacagcctgcaggacctccaccacttgaacctgggcgcggccggcgcgcacgactttttctcggcagggcagcaggccgccgccgcagctgcgatgcacggcctggctagcatcgacagtgcgtcgctcgagcacagcaccggctccaactccgtcgtctacaacggcggggtcggcgatagcaacggcgccagcgccgttggcagcggcggtggctacatgatgccgatgagcgctgccggagcaaccactacatcggcaatggtgagccacgagcagatgcatgcacgggcctacgacgaagccaagcaggctgctcagatggggtacgagagctacctggtgaacgcggagaacaatggtggcggaaggatgtctgcatgggggaccgtcgtctctgcagccgcggcggcagcagcaagcagcaacgacaacattgccgccgacgtcggccatggcggcgcgcagctcttcagtgtctggaacgacacttaa

SEQ ID NO: 10 - protein, 709 aa, Zea mays
MATVNNWLAFSLSPQELPPSQTTDSTLISAATADHVSGDVCFNIPQDWSMRGSELSALVAEPKLEDFLGGISFSEQHHKSNCNLIPSTSSTVCYASSAASTGYHHQLYQPTSSALHFADSVMVASSAGVHDGGSMLSAAAANGVAGAASANGGGIGLSMIKNWLRSQPAPMQPRAAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAATTGLYHPYAQQPMRGGGWCKQEQDHAVIAAAHSLQDLHHLNLGAAGAHDFFSAGQQAAAAAAMHGLASIDSASLEHSTGSNSVVYNGGVGDSNGASAVGSGGGYMMPMSAAGATTTSAMVSHEQMHARAYDEAKQAAQMGYESYLVNAENNGGGRMSAWGTVVSAAAAAAASSNDNIAADVGHGGAQLFSVWNDT

SEQ ID NO: 11 - DNA, 2040 bp, Zea mays
  source 1..2040: mol_type = genomic DNA
  CDS 1..2040
atggcttcagcgaacaactggctgggcttctcgctctcgggccaggataacccgcagcctaaccaggatagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctctccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtgcccagcggcgccgccagcggagcggcctcggagagcacatcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagggaagggcagagtcgcaagggtaggcaagtttaccttggtggctatgacaaggaggacaaggcagcaagggcttatgatttggcagctctcaagtattggggcactacgacaacaacaaatttccctataagcaactacgaaaaggagctagaagaaatgaaacatatgactagacaggagtacattgcatacctaagaagaaatagcagtggattttctcgtggggcgtcaaagtatcgtggagtaactagacatcatcagcatgggagatggcaagcaaggatagggagagttgcaggaaacaaggatctctacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggtgcggccaggcgcctcaaggacgccgtggaccacgtggaggccggcgccaccatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcgcacagcctgcaggacctccagcagctgcacctcggcagcgcggcccacaacttcttccaggcgtcgtcgagctccacagtctacaacggcggcgccggcgccagtggtgggtaccagggcctcggtggtggcagctctttcctcatgccgtcgagcactgtcgtggcggcggccgaccaggggcacagcagcacggccaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtga

SEQ ID NO: 12 - protein, 679 aa, Zea mays
MASANNWLGFSLSGQDNPQPNQDSSPAAGIDISGASDFYGLPTQQGSDGHLGVPGLRDDHASYGIMEAYNRVPQETQDWNMRGLDYNGGGSELSMLVGSSGGGGGNGKRAVEDSEPKLEDFLGGNSFVSDQDQSGGYLFSGVPIASSANSNSGSNTMELSMIKTWLRNNQVAQPQPPAPHQPQPEEMSTDASGSSFGCSDSMGRNSMVAAGGSSQSLALSMSTGSHLPMVVPSGAASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGAVISQLAEAGMGGYASYGHHGWPTIAFQQPSPLSVHYPYGQPSRGWCKPEQDAAAAAAHSLQDLQQLHLGSAAHNFFQASSSSTVYNGGAGASGGYQGLGGGSSFLMPSSTVVAAADQGHSSTANQGSTCSYGDDHQEGKLIGYDAAMVATAAGGDPYAAARNGYQFSQGSGSTVSIARANGYANNWSSPFNNGMG

SEQ ID NO: 13 - DNA, 2088 bp, Oryza sativa
  source 1..2088: mol_type = genomic DNA
  CDS 1..2088
atggccaccatgaacaactggctggccttctccctctccccgcaggatcagctcccgccgtctcagaccaactccactctcatctccgccgccgccaccaccaccaccgccggcgactcctccaccggcgacgtctgcttcaacatcccccaagattggagcatgaggggatcggagctctcggcgctcgtcgccgagccgaagctggaggacttcctcggcggcatctccttctcggagcagcagcatcatcacggcggcaagggcggcgtgatcccgagcagcgccgccgcttgctacgcgagctccggcagcagcgtcggctacctgtaccctcctccaagctcatcctcgctccagttcgccgactccgtcatggtggccacctcctcgcccgtcgtcgcccacgacggcgtcagcggcggcggcatggtgagcgccgccgccgccgcggcggccagtggcaacggcggcattggcctgtccatgatcaagaactggctccggagccagccggcgccgcagccggcgcaggcgctgtctctgtccatgaacatggcggggacgacgacggcgcagggcggcggcgccatggcgctcctcgccggcgcaggggagcgaggccggacgacgcccgcgtcagagagcctgtccacgtcggcgcacggagcgacgacggcgacgatggctggtggtcgcaaggagattaacgaggaaggcagcggcagcgccggcgccgtggttgccgtcggctcggagtcaggcggcagcggcgccgtggtggaggccggcgcggcggcggcggcggcgaggaagtccgtcgacacgttcggccagagaacatcgatctaccgcggcgtgacaaggcatagatggacagggaggtatgaggctcatctttgggacaacagctgcagaagagagggccaaactcgcaagggtcgtcaagtctatctaggtggttatgacaaagaggaaaaagctgctagagcttatgatttggctgctctcaaatactggggcccgacgacgacgacaaattttccggtaaataactatgaaaaggagctggaggagatgaagcacatgacaaggcaggagttcgtagcctctttgagaaggaagagcagtggtttctccagaggtgcatccatttaccgtggagtaactaggcatcaccagcatgggagatggcaagcaaggataggaagagttgcagggaacaaggacctctacttgggcaccttcagcacgcaggaggaggcggcggaggcgtacgacatcgcggcgatcaagttccgggggctcaacgccgtcaccaacttcgacatgagccgctacgacgtcaagagcatcctcgacagcgctgccctccccgtcggcaccgccgccaagcgcctcaaggacgccgaggccgccgccgcctacgacgtcggccgcatcgcctcgcacctcggcggcgacggcgcctacgccgcgcattacggccaccaccaccactcggccgccgccgcctggccgaccatcgcgttccaggcggcggcggcgccgccgccgcacgccgccgggctttaccacccgtacgcgcagccgctgcgtgggtggtgcaagcaggagcaggaccacgccgtgatcgcggcggcgcacagcctgcaggatctccaccacctcaacctcggcgccgccgccgccgcgcatgacttcttctcgcaggcgatgcagcagcagcacggcctcggcagcatcgacaacgcgtcgctcgagcacagcaccggctccaactccgtcgtctacaacggcgacaatggcggcggaggcggcggctacatcatggcgccgatgagcgccgtgtcggccacggccaccgcggtggcgagcagccacgatcacggcggcgacggcgggaagcaggtgcagatggggtacgacagctacctcgtcggcgcagacgcctacggcggcggcggcgccgggaggatgccatcctgggcgatgacgccggcgtcggcgccggccgccacgagcagcagcgacatgaccggagtctgccatggcgcacagctcttcagcgtctggaacgacacataa

SEQ ID NO: 14 - protein, 695 aa, Oryza sativa
MATMNNWLAFSLSPQDQLPPSQTNSTLISAAATTTTAGDSSTGDVCFNIPQDWSMRGSELSALVAEPKLEDFLGGISFSEQQHHHGGKGGVIPSSAAACYASSGSSVGYLYPPPSSSSLQFADSVMVATSSPVVAHDGVSGGGMVSAAAAAAASGNGGIGLSMIKNWLRSQPAPQPAQALSLSMNMAGTTTAQGGGAMALLAGAGERGRTTPASESLSTSAHGATTATMAGGRKEINEEGSGSAGAVVAVGSESGGSGAVVEAGAAAAAARKSVDTFGQRTSIYRGVTRHRWTGRYEAHLWDNSCRREGQTRKGRQVYLGGYDKEEKAARAYDLAALKYWGPTTTTNFPVNNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSAALPVGTAAKRLKDAEAAAAYDVGRIASHLGGDGAYAAHYGHHHHSAAAAWPTIAFQAAAAPPPHAAGLYHPYAQPLRGWCKQEQDHAVIAAAHSLQDLHHLNLGAAAAAHDFFSQAMQQQHGLGSIDNASLEHSTGSNSVVYNGDNGGGGGGYIMAPMSAVSATATAVASSHDHGGDGGKQVQMGYDSYLVGADAYGGGGAGRMPSWAMTPASAPAATSSSDMTGVCHGAQLFSVWNDT

SEQ ID NO: 15 - DNA, 1680 bp, Oryza sativa
  source 1..1680: mol_type = genomic DNA
  CDS 1..1680
atggcctccatcaccaactggctcggcttctcctcctcctccttctccggcgccggcgccgaccccgtcctgccccacccgccgctgcaagagtgggggagcgcttatgagggcggcggcacggtggcggccgccggcggggaggagacggcggcgccgaagctggaggacttcctcggcatgcaggtgcagcaggagacggccgccgcggcggcggggcacggccgtggaggcagctcgtcggtcgttgggctgtccatgatcaagaactggctacgcagccagccgccgcccgcggtggttgggggagaagacgctatgatggcgctcgcggtgtcgacgtcggcgtcgccgccggtggacgcgacggtgccggcctgcatttcgccggatgggatggggtcgaaggcggccgacggcggcggcgcggccgaggcggcggcggcggcggcggcgcagaggatgaaggcggccatggacacgttcgggcagcggacgtccatctaccggggtgtcaccaagcacaggtggacaggaaggtatgaagcccatctttgggataacagctgcagaagagaaggtcagactcgcaaaggcagacaagtatatcttggaggatatgataaggaagaaaaagctgctagggcttatgatttggctgcccttaaatactggggcactacaacgacgacgaattttccggtaagcaactacgaaaaagagttggatgaaatgaagcacatgaataggcaggaatttgttgcatcccttagaagaaaaagcagtggattttcacgtggtgcttccatatatcgtggtgttacaagacaccatcagcatggaaggtggcaagcaaggataggacgggtggcaggaaacaaggatctgtatttgggcacatttggcacccaagaggaagctgcagaggcatatgatatcgctgcaatcaaattccgtggtctcaatgctgtgacaaactttgacatgagccggtacgatgtcaagagcatcattgaaagcagcaatctcccaattggtactggaaccacccggcgattgaaggactcctctgatcacactgataatgtcatggacatcaatgtcaataccgaacccaataatgtggtatcatcccacttcaccaatggggttggcaactatggttcgcagcattatggttacaatggatggtcgccaattagcatgcagccgatcccctcgcagtacgccaacggccagcccagggcatggttgaaacaagagcaggacagctctgtggttacagcggcgcagaacctgcacaatctacatcattttagttccttgggctacacccacaacttcttccagcaatctgatgttccagacgtcacaggtttcgttgatgcgccttcgaggtccagtgactcatactccttcaggtacaatggaacaaatggctttcatggtctcccgggtggaatcagctatgctatgccggttgcgacagcggtggaccaaggtcagggcatccatggctatggagaagatggtgtggcaggcattgacaccacacatgacctgtatggcagccgtaatgtgtactacctttccgagggttcgcttcttgccgatgtcgaaaaagaaggcgactatggccaatctgtggggggcaacagctgggttttgccgacaccgtag

SEQ ID NO: 16 - protein, 559 aa, Oryza sativa
MASITNWLGFSSSSFSGAGADPVLPHPPLQEWGSAYEGGGTVAAAGGEETAAPKLEDFLGMQVQQETAAAAAGHGRGGSSSVVGLSMIKNWLRSQPPPAVVGGEDAMMALAVSTSASPPVDATVPACISPDGMGSKAADGGGAAEAAAAAAAQRMKAAMDTFGQRTSIYRGVTKHRWTGRYEAHLWDNSCRREGQTRKGRQVYLGGYDKEEKAARAYDLAALKYWGTTTTTNFPVSNYEKELDEMKHMNRQEFVASLRRKSSGFSRGASIYRGVTRHHQHGRWQARIGRVAGNKDLYLGTFGTQEEAAEAYDIAAIKFRGLNAVTNFDMSRYDVKSIIESSNLPIGTGTTRRLKDSSDHTDNVMDINVNTEPNNVVSSHFTNGVGNYGSQHYGYNGWSPISMQPIPSQYANGQPRAWLKQEQDSSVVTAAQNLHNLHHFSSLGYTHNFFQQSDVPDVTGFVDAPSRSSDSYSFRYNGTNGFHGLPGGISYAMPVATAVDQGQGIHGYGEDGVAGIDTTHDLYGSRNVYYLSEGSLLADVEKEGDYGQSVGGNSWVLPTP

SEQ ID NO: 17 - DNA, 2112 bp, Oryza sativa
  source 1..2112: mol_type = genomic DNA
  CDS 1..2112
atggcttctgcaaacaactggctgggcttctcgctctccggccaagagaatccgcagcctcaccaggatagctcgcctccggcagccatcgacgtctccggcgccggcgacttctatggcctgccgacgtcgcagccgacggcggccgacgcgcacctcggcgtggcggggcatcatcacaacgcctcgtatggcatcatggaggccttcaataggggagctcaagaggcacaagattggaacatgagggggctggactacaacggcggcgcctcggagctgtcgatgctcgtcggctccagcggcggcaagagggcggcggcggtggaggagaccgagccgaagctggaggacttcctcggcggcaactcgttcgtctccgagcaagatcatcacgcggcggggggcttcctcttctccggcgtcccgatggccagcagcaccaacagcaacagcgggagcaacactatggagctctccatgatcaagacctggctccggaacaacggccaggtgcccgccggccaccagccgcagcagcagcagccggcggccgcggccgccgccgcgcagcagcaggcgcacgaggcggcggagatgagcaccgacgcgagcgcgagcagcttcgggtgctcctccgacgcgatggggaggagtaacaacggcggcgcggtctcggcggcggccggcgggacgagctcgcagagcctggcgctctcgatgagcacgggctcgcactcgcacctgcctatcgtcgtcgccggcggcgggaacgccagcggcggagcggccgagagcacatcgtcggagaacaagcgggccagcggcgccatggattcgccgggcggtggcgcgatagaggccgtgccgaggaagtccatcgacacgttcgggcaaaggacctcgatatatcgaggtgtaacaaggcatagatggacagggcgatatgaggctcatctctgggataatagctgtagaagagaagggcagagtcgcaagggtaggcaagtttatcttggtggctatgacaaggaggataaagcagcgagagcttatgatttggcagctctgaagtattggggcacaacaacaacaacaaatttcccaataagtaactatgaaaaagagctagatgaaatgaaacatatgaccaggcaggagtatattgcatacctaagaaggaatagcagtggattttctcgtggtgcatcgaaatatcgtggtgtaaccaggcaccatcagcatgggagatggcaagcaaggatagggagggttgcaggaaacaaggacctctacttaggcaccttcagcaccgaggaggaggcggcggaggcgtacgacatcgcggcgatcaagttccgggggctcaacgccgtcaccaactttgacatgagccgctacgacgtcaagagcatcctggagagcagcacgctgccggtgggcggcgcggcgaggcggctgaaggaggcggcggaccacgcggaggcggccggcgccaccatctggcgcgccgccgacatggacggcgccggcgtcatctccggcctggccgacgtcgggatgggcgcctacgccgcctcgtaccaccaccaccaccaccacggctggccgaccatcgcgttccagcagccgccgccgctcgccgtgcactacccgtacggccaggcgccggcggcgccgtcgcgcgggtggtgcaagcccgagcaggacgccgccgtcgctgccgccgcgcacagcctccaggacctccagcagctgcacctcggcagcgccgccgcccacaacttcttccaggcgtcgtcgagctcgacggtctacaacggcggcggcggcgggtaccagggcctcggtggcaacgccttcttgatgccggcgagcaccgtcgtggccgaccaggggcacagcagcacggccaccaaccatggaaacacctgcagctacggcaacgaggagcaggggaagctcatcgggtacgacgccatggcgatggcgagcggcgccgccggcggcgggtaccagctgtcgcagggctcggcgtcgacggtgagcatcgcgagggcgaacggctactcggccaactggagctcgcctttcaatggcgccatgggatga

SEQ ID NO: 18 - protein, 703 aa, Oryza sativa
MASANNWLGFSLSGQENPQPHQDSSPPAAIDVSGAGDFYGLPTSQPTAADAHLGVAGHHHNASYGIMEAFNRGAQEAQDWNMRGLDYNGGASELSMLVGSSGGKRAAAVEETEPKLEDFLGGNSFVSEQDHHAAGGFLFSGVPMASSTNSNSGSNTMELSMIKTWLRNNGQVPAGHQPQQQQPAAAAAAAQQQAHEAAEMSTDASASSFGCSSDAMGRSNNGGAVSAAAGGTSSQSLALSMSTGSHSHLPIVVAGGGNASGGAAESTSSENKRASGAMDSPGGGAIEAVPRKSIDTFGQRTSIYRGVTRHRWTGRYEAHLWDNSCRREGQSRKGRQVYLGGYDKEDKAARAYDLAALKYWGTTTTTNFPISNYEKELDEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKEAADHAEAAGATIWRAADMDGAGVISGLADVGMGAYAASYHHHHHHGWPTIAFQQPPPLAVHYPYGQAPAAPSRGWCKPEQDAAVAAAAHSLQDLQQLHLGSAAAHNFFQASSSSTVYNGGGGGYQGLGGNAFLMPASTVVADQGHSSTATNHGNTCSYGNEEQGKLIGYDAMAMASGAAGGGYQLSQGSASTVSIARANGYSANWSSPFNGAMG

SEQ ID NO: 19 - DNA, 1977 bp, Oryza sativa
  source 1..1977: mol_type = genomic DNA
  CDS 1..1977
atggcttctgcagataactggctaggcttctcgctctccggccaaggcaacccacagcatcaccagaacggctcgccgtctgccgccggcgacgccgccatcgacatctccggctcaggcgacttctatggtctgccaacgccggacgcacaccacatcggcatggcgggcgaagacgcgccctatggcgtcatggatgctttcaacagaggcacccatgaaacccaagattgggcgatgaggggtttggactacggcggcggctcctccgacctctcgatgctcgtcggctcgagcggcggcgggaggaggacggtggccggcgacggcgtcggcgaggcgccgaagctggagaacttcctcgacggcaactcattctccgacgtgcacggccaagccgccggcgggtacctctactccggaagcgctgtcggcggcgccggtggttacagtaacggcggatgcggcggcggaaccatagagctgtccatgatcaagacgtggctccggagcaaccagtcgcagcagcagccatcgccgccgcagcacgctgatcagggcatgagcaccgacgccagcgcgagcagctacgcgtgctccgacgtgctggtggggagctgcggcggcggcggcgccgggggcacggcgagctcgcatgggcagggcctggcgctgtcgatgagcacggggtcggtggccgccgccggagggggcggcgccgtcgtcgcggccgagagctcgtcgtcggagaacaagcgggtggattcgccgggcggcgccgtggacggcgccgtcccgaggaaatccatcgacaccttcgggcaaaggacgtctatataccgaggtgtaacaaggcatagatggacaggaagatatgaagctcatctgtgggataatagctgtaggagagaaggccaaagtcgcaaggggagacaggtttatttgggcggttatgacaaagaagataaggcggctcgggcttatgatttggcagctctaaaatactggggcacgaccacaacaacaaatttcccaatgagtaattatgaaaaggagctagaggaaatgaaacacatgaccaggcaggagtacattgcacatcttagaaggaatagcagtggattttctcgtggtgcatccaaatatcgtggtgttactaggcatcatcagcatgggagatggcaggcaaggatagggcgagttgcaggcaacaaggatatctacctaggcaccttcagcaccgaggaggaggccgccgaggcgtacgacatcgccgccatcaagttccgcgggctcaacgccgtcaccaacttcgacatgagccggtacgacgtcaagagcatcctggacagcagcacgctgccggtcggcggcgcggcgcggcggctcaaggaggcggaggtcgccgccgccgccgcgggcggcggcgtgatcgtctcccacctggccgacggcggtgtgggtgggtactactacgggtgcggcccgaccatcgcgttcggcggcggcggccagcagccggcgccgctcgccgtgcactacccgtcgtacggccaggccagcgggtggtgcaagccggagcaggacgcggtgatcgcggccgggcactgcgcgacggacctccagcacctgcacctcgggagcggcggcgccgccgccacccacaacttcttccagcagccggcgtcaagctcggccgtctacggcaacggcggcggcggcggcggcaacgcgttcatgatgccgatgggcgccgtggtggccgccgccgatcacggcgggcagagcagcgcctacggcggtggcgacgagagcgggaggctcgtcgtggggtacgacggcgtcgtcgacccgtacgcggccatgagaagcgcgtacgagctctcgcagggctcgtcgtcgtcgtcggtgagcgtcgcgaaggcggcgaacgggtacccggacaactggagctcgccgttcaacggcatgggatga

SEQ ID NO: 20 - protein, 658 aa, Oryza sativa
MASADNWLGFSLSGQGNPQHHQNGSPSAAGDAAIDISGSGDFYGLPTPDAHHIGMAGEDAPYGVMDAFNRGTHETQDWAMRGLDYGGGSSDLSMLVGSSGGGRRTVAGDGVGEAPKLENFLDGNSFSDVHGQAAGGYLYSGSAVGGAGGYSNGGCGGGTIELSMIKTWLRSNQSQQQPSPPQHADQGMSTDASASSYACSDVLVGSCGGGGAGGTASSHGQGLALSMSTGSVAAAGGGGAVVAAESSSSENKRVDSPGGAVDGAVPRKSIDTFGQRTSIYRGVTRHRWTGRYEAHLWDNSCRREGQSRKGRQVYLGGYDKEDKAARAYDLAALKYWGTTTTTNFPMSNYEKELEEMKHMTRQEYIAHLRRNSSGFSRGASKYRGVTRHHQHGRWQARIGRVAGNKDIYLGTFSTEEEAAEAYDIAAIKFRGLNAVTNFDMSRYDVKSILDSSTLPVGGAARRLKEAEVAAAAAGGGVIVSHLADGGVGGYYYGCGPTIAFGGGGQQPAPLAVHYPSYGQASGWCKPEQDAVIAAGHCATDLQHLHLGSGGAAATHNFFQQPASSSAVYGNGGGGGGNAFMMPMGAVVAAADHGGQSSAYGGGDESGRLVVGYDGVVDPYAAMRSAYELSQGSSSSSVSVAKAANGYPDNWSSPFNGMG

SEQ ID NO: 21 - DNA, 1755 bp, Arabidopsis thaliana
  source 1..1755: mol_type = genomic DNA
  CDS 1..1755
atgaactcgatgaataactggttaggcttctctctctctcctcatgatcaaaatcatcaccgtacggatgttgactcctccaccaccagaaccgccgtagatgttgccggagggtactgttttgatctggccgctccctccgatgaatcttctgccgttcaaacatcttttctttctcctttcggtgtcaccctcgaagctttcaccagagacaataatagtcactcccgagattgggacatcaatggtggtgcatgcaatacattaaccaataacgaacaaaatggaccaaagcttgagaatttcctcggccgcaccaccacgatttacaataccaacgagaccgttgtagatggaaatggcgattgtggaggaggagacggtggtggtggcggctcactaggcctttcgatgataaaaacatggctgagtaatcattcggttgctaatgctaatcatcaagacaatggtaacggtgcacgaggcttgtccctctctatgaattcatctactagtgatagcaacaactacaacaacaatgatgatgtcgtccaagagaagactattgttgatgtcgtagaaactacaccgaagaaaactattgagagttttggacaaaggacgtctatataccgcggtgttacaaggcatcggtggacaggtagatacgaggcacatttatgggacaatagttgcaaaagagaaggccagactcgcaaaggaagacaagtttatctgggaggttatgacaaagaagaaaaagcagctagggcttacgatttagccgcactaaagtattggggacccaccactactactaacttccccttgagtgaatatgagaaagaggtagaagagatgaagcacatgacgaggcaagagtatgttgcctctctgcgcaggaaaagtagtggtttctctcgtggtgcatcgatttatcgaggagtaacaaggcatcaccaacatggaaggtggcaagctaggatcggaagagtcgccggtaacaaagacctctacttgggaactttcggcacacaggaagaggctgctgaggcttatgacattgcagccattaaattcagaggattaagcgcagtgactaacttcgacatgaacagatacaatgttaaagcaatcctcgagagcccgagtctacctattggtagttctgcgaaacgtctcaaggacgttaacaatccggttccagctatgatgattagtaataacgtttcagagagtgcaaataatgttagcggttggcaaaacactgcgtttcagcatcatcagggaatggatttgagcttattgcagcaacagcaggagaggtacgttggttattacaatggaggaaacttgtctaccgagagtactagggtttgtttcaaacaagaggaggaacaacaacacttcttgagaaactcgccgagtcacatgactaatgttgatcatcatagctcgacctctgatgattctgttaccgtttgtggaaatgttgttagttatggtggttatcaaggattcgcaatccctgttggaacatcggttaattacgatccctttactgctgctgagattgcttacaacgcaagaaatcattattactatgctcagcatcagcaacaacagcagattcagcagtcgccgggaggagattttccggtggcgatttcgaataaccatagctctaacatgtactttcacggggaaggtggtggagaaggggctccaacgttttcagtttggaacgacacttag

SEQ ID NO: 22 - protein, 584 aa, Arabidopsis thaliana
MNSMNNWLGFSLSPHDQNHHRTDVDSSTTRTAVDVAGGYCFDLAAPSDESSAVQTSFLSPFGVTLEAFTRDNNSHSRDWDINGGACNTLTNNEQNGPKLENFLGRTTTIYNTNETVVDGNGDCGGGDGGGGGSLGLSMIKTWLSNHSVANANHQDNGNGARGLSLSMNSSTSDSNNYNNNDDVVQEKTIVDVVETTPKKTIESFGQRTSIYRGVTRHRWTGRYEAHLWDNSCKREGQTRKGRQVYLGGYDKEEKAARAYDLAALKYWGPTTTTNFPLSEYEKEVEEMKHMTRQEYVASLRRKSSGFSRGASIYRGVTRHHQHGRWQARIGRVAGNKDLYLGTFGTQEEAAEAYDIAAIKFRGLSAVTNFDMNRYNVKAILESPSLPIGSSAKRLKDVNNPVPAMMISNNVSESANNVSGWQNTAFQHHQGMDLSLLQQQQERYVGYYNGGNLSTESTRVCFKQEEEQQHFLRNSPSHMTNVDHHSSTSDDSVTVCGNVVSYGGYQGFAIPVGTSVNYDPFTAAEIAYNARNHYYYAQHQQQQQIQQSPGGDFPVAISNNHSSNMYFHGEGGGEGAPTFSVWNDT

SEQ ID NO: 23 - DNA, 1740 bp, Brassica napus
  source 1..1740: mol_type = genomic DNA
  CDS 1..1740
atgaataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaattag

SEQ ID NO: 24 - protein, 579 aa, Brassica napus
MNNNWLGFSLSPYEQNHHRKDVYSSTTTTVVDVAGEYCYDPTAASDESSAIQTSFPSPFGVVVDAFTRDNNSHSRDWDINGCACNNIHNDEQDGPKLENFLGRTTTIYNTNENVGDGSGSGCYGGGDGGGGSLGLSMIKTWLRNQPVDNVDNQENGNAAKGLSLSMNSSTSCDNNNDSNNNVVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEVEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENSASGWQNAAVQHHQGVDLSLLHQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQQSPGGDFPAAMTNNVGSNMYYHGEGGGEVAPTFTVWNDN

SEQ ID NO: 25 - DNA, 1740 bp, Brassica napus
  source 1..1740: mol_type = genomic DNA
  CDS 1..1740
atgaataataactggttaggcttttctctctctccttatgaacaaaatcaccatcgtaaggacgtctgctcttccaccaccacaaccgccgtagatgtcgccggagagtactgttacgatccgaccgctgcctccgatgagtcttcagccatccaaacatcgtttccttctccctttggtgtcgtcctcgatgctttcaccagagacaacaatagtcactcccgagattgggacatcaatggtagtgcatgtaataacatccacaatgatgagcaagatggaccaaaacttgagaatttccttggccgcaccaccacgatttacaacaccaacgaaaacgttggagatatcgatggaagtgggtgttatggaggaggagacggtggtggtggctcactaggactttcgatgataaagacatggctgagaaatcaacccgtggataatgttgataatcaagaaaatggcaatggtgcaaaaggcctgtccctctcaatgaactcatctacttcttgtgataacaacaactacagcagtaacaaccttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaacgagaaggccaaacgcgcaaaggaagacaagtttatttgggaggttatgacaaagaagaaaaagcagctagggcttatgatttagccgcactcaagtattggggaaccaccactactactaacttccccatgagcgaatatgagaaagagatagaagagatgaagcacatgacaaggcaagagtatgttgcctcacttcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaataatgctagcggttggcaaaacgctgcggttcagcatcatcagggagtagatttgagcttattgcagcaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcagatgattcggttactgtttgtggaaatgttgttggttatggtggttatcaaggatttgcagccccggttaactgcgatgcctacgctgctagtgagtttgactataacgcaagaaaccattattactttgctcagcagcagcagacccagcattcgccaggaggagattttcccgcggcaatgacgaataatgttggctctaatatgtattaccatggggaaggtggtggagaagttgctccaacatttacagtttggaacgacaattag

SEQ ID NO: 26 - protein, 579 aa, Brassica napus
MNNNWLGFSLSPYEQNHHRKDVCSSTTTTAVDVAGEYCYDPTAASDESSAIQTSFPSPFGVVLDAFTRDNNSHSRDWDINGSACNNIHNDEQDGPKLENFLGRTTTIYNTNENVGDIDGSGCYGGGDGGGGSLGLSMIKTWLRNQPVDNVDNQENGNGAKGLSLSMNSSTSCDNNNYSSNNLVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEIEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENNASGWQNAAVQHHQGVDLSLLQQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQHSPGGDFPAAMTNNVGSNMYYHGEGGGEVAPTFTVWNDN

SEQ ID NO: 27 - DNA, 2082 bp, Sorghum bicolor
  source 1..2082: mol_type = genomic DNA
  CDS 1..2082
atggcttcgacgaacaaccactggctgggtttctcgctctcgggccaggataacccgcagcctaatcatcaggacagctcgcctgccgccgccggcatcgacatctccggcgccagcgacttctatggcttgcccacgcagcagggctccgacgggaatctcggcgtgccgggcctgcgggacgatcacgcttcttatggcatcatggaggccttcaacagggttcctcaagaaacccaagattggaacatgaggggattggactacaacggcggtggctcggaactctcgatgcttgtggggtccagcggcggcggcgggggcggcggcaagagggccgtggaagacagcgagcccaagctcgaagatttcctcggcggcaactcgttcgtctccgagcatgatcagtccggcggttacctgttctctggagtcccgatggccagcagcaccaacagcaacagcgggagcaacaccatggagctctccatgatcaagacctggctccggaacaaccaggtgccccagccgcagccgccagcagctccgcatcaggcgccgcagactgaggagatgagcaccgacgccaacgccagcgccagcagctttggctgctcggattcgatggggaggaacggcacggtggcggctgctgggagctcccagagcctggcgctctcgatgagcacgggctcgcacctgccgatggttgtggccggcggcggcgccagcggagcggcctcggagagcacgtcatcggagaacaagcgagcgagcggcgccatggattcgcccggcagcgcggtagaagccgtcccgaggaagtccatcgacacgttcgggcaaaggacctctatatatcgaggtgtaacaagacatagatggacagggcgatatgaggctcatctatgggataatagttgtagaagagaagggcagagtcgcaagggtaggcaagtttaccttggtggctatgacaaggaagacaaggcagcaagggcttatgatttggcagctctcaagtattggggcactactacaacaacaaatttccctataagcaactatgaaaaggagctagaggaaatgaaacatatgactaggcaggagtatattgcatacctaagaagaaatagcagtggattttctcgtggcgcatcaaaatatcgtggagtaactagacatcatcagcatgggagatggcaagcaaggatagggagagttgcaggaaacaaggatctctacttgggcacattcagcaccgaggaggaggcggcggaggcctacgacatcgccgcgatcaagttccgcggtctgaacgccgtcaccaacttcgacatgagccgctacgacgtcaagagcatcctcgagagcagcacgctgcctgtcggcggcgcggccaggcgcctcaaggatgccgtggaccacgtggaggccggcgccaccatctggcgcgccgacatggacggcggcgtgatctcccagctcgccgaagccgggatgggcggctacgcctcgtacgggcaccacgcctggccgaccatcgcgttccagcagccgtcgccgctctccgtccactacccgtacgggcagccgccgtcccgcgggtggtgcaagcccgagcaggacgcggccgtcgccgccgccgcgcacagcctgcaggacctccagcagctgcacctcggcagcgcggcacacaacttcttccaggcgtcgtcgagctcggcagtctacaacagcggcggcggcggcgctagcggcgggtaccaccagggcctcggtggcggcagcagctccttcctcatgccgtcgagcactgtcgtggcgggggccgaccaggggcacagcagcagcacggccaaccaggggagcacgtgcagctacggggacgatcaccaggaagggaagctcatcgggtacgacgccatggtggcggcgaccgcagccggcggggacccgtacgccgcggcgaggagcgggtaccagttctcgtcgcagggctcgggatccacggtgagcatcgcgagggcgaacgggtactctaacaactggagctctcctttcaacggcggcatggggtga

SEQ ID NO: 28 - protein, 693 aa, Sorghum bicolor
MASTNNHWLGFSLSGQDNPQPNHQDSSPAAAGIDISGASDFYGLPTQQGSDGNLGVPGLRDDHASYGIMEAFNRVPQETQDWNMRGLDYNGGGSELSMLVGSSGGGGGGGKRAVEDSEPKLEDFLGGNSFVSEHDQSGGYLFSGVPMASSTNSNSGSNTMELSMIKTWLRNNQVPQPQPPAAPHQAPQTEEMSTDANASASSFGCSDSMGRNGTVAAAGSSQSLALSMSTGSHLPMVVAGGGASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGGVISQLAEAGMGGYASYGHHAWPTIAFQQPSPLSVHYPYGQPPSRGWCKPEQDAAVAAAAHSLQDLQQLHLGSAAHNFFQASSSSAVYNSGGGGASGGYHQGLGGGSSSFLMPSSTVVAGADQGHSSSTANQGSTCSYGDDHQEGKLIGYDAMVAATAAGGDPYAAARSGYQFSSQGSGSTVSIARANGYSNNWSSPFNGGMG

SEQ ID NO: 29 - DNA, 508 bp, Zea mays
  source 1..508: mol_type = genomic DNA
ctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgct

SEQ ID NO: 30 - DNA, 665 bp, synthetic construct
  misc_feature 1..665: note = Promoter construct comprising Zea mays Rab17promoter and attB1 site
  source 1..665: mol_type = other DNA
ctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaagcttggtcacccggtccgggcctagaaggccagcttcaagtttgtacaaaaaagcaggct

SEQ ID NO: 31 - DNA, 24 bp, synthetic construct
  misc_feature 1..24: note = attachment B1 site
  source 1..24: mol_type = other DNA
caagtttgtacaaaaaagcaggct

SEQ ID NO: 32 - DNA, 24 bp, synthetic construct
  misc_feature 1..24: note = attachment B2 site
  source 1..24: mol_type = other DNA
acccagctttcttgtacaaagtgg

SEQ ID NO: 33 - DNA, 22 bp, synthetic construct
  misc_feature 1..22: note = attachment B3 site
  source 1..22: mol_type = other DNA
acaactttgtataataaagttg

SEQ ID NO: 34 - DNA, 22 bp, synthetic construct
  misc_feature 1..22: note = attachment B4 site
  source 1..22: mol_type = other DNA
acaactttgtatagaaaagttg

SEQ ID NO: 35 - DNA, 95 bp, Zea mays
  source 1..95: mol_type = genomic DNA
tcaccaccggcgagccacatcgagaacacgatcgagcacacaagcacgaagactcgtttaggagaaaccacaaaccaccaagccgtgcaagcacc

SEQ ID NO: 36 - DNA, 133 bp, synthetic construct
  misc_feature 1..133: note = Plasmid linker sequence
  source 1..133: mol_type = other DNA
tcaccaccggcgagccacatcgagaacacgatcgagcacacaagcacgaagactcgtttaggagaaaccacaaaccaccaagccgtgcaagcaccaagcttggtcacccggtccgggcctagaaggccagctt

SEQ ID NO: 37 - DNA, 61 bp, synthetic construct
  misc_feature 1..61: note = Plasmid linker sequence
  source 1..61: mol_type = other DNA
tcgaaggagatagaaccaattctctaaggaaatacttaaccatggtcgactggatccaaca

SEQ ID NO: 38 - DNA, 318 bp, synthetic construct
  misc_feature 1..318: note = PinII terminator
  source 1..318: mol_type = other DNA
agacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggc

SEQ ID NO: 39 - DNA, 16 bp, synthetic construct
  misc_feature 1..16: note = Plasmid linker sequence
  source 1..16: mol_type = other DNA
gtttcgagatatctag

SEQ ID NO: 40 - DNA, 14 bp, synthetic construct
  misc_feature 1..14: note = Plasmid linker sequence
  source 1..14: mol_type = other DNA
ccgttaacggatcc

SEQ ID NO: 41 - DNA, 1272 bp, synthetic construct
  misc_feature 1..1272: note = Maize optimized FLP coding sequence
  source 1..1272: mol_type = other DNA
  CDS 1..1272
atgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctcggcgtgatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctga

SEQ ID NO: 42 - protein, 423 aa, synthetic construct
  REGION 1..423: note = FLP
MPQFDILCKTPPKVLVRQFVERFERPSGEKIALCAAELTYLCWMITHNGTAIKRATFMSYNTIISNSLSFDIVNKSLQFKYKTQKATILEASLKKLIPAWEFTIIPYYGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYSIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 43 - DNA, 1032 bp, synthetic construct
  misc_feature 1..1032: note = Maize optimized Cre coding sequence
  source 1..1032: mol_type = other DNA
  CDS 1..1032
atgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagctcgcgggctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactga

SEQ ID NO: 44 - protein, 343 aa, synthetic construct
  REGION 1..343: note = Cre
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 45 - DNA, 1198 bp, synthetic construct
  misc_feature 1..1198: note = Expression cassette comprising Zea mays rab17promoter, attB1 site, and FLPm coding sequence
  source 1..1198: mol_type = other DNA
ctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaagcttggtcacccggtccgggcctagaaggccagcttcaagtttgtacaaaaaagcaggcttcgaaggagatagaaccaattctctaaggaaatacttaaccatggtcgactggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatca

SEQ ID NO: 46 - DNA, 2370 bp, synthetic construct
  misc_feature 1..2370: note = Plasmid PHP31004
  source 1..2370: mol_type = other DNA
ctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaagcttggtcacccggtccgggcctagaaggccagcttcaagtttgtacaaaaaagcaggcttcgaaggagatagaaccaattctctaaggaaatacttaaccatggtcgactggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctcggcgtgatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagtttcgagatatctagacccagctttcttgtacaaagtggccgttaacggatccagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggc

SEQ ID NO: 47 - DNA, 2383 bp, synthetic construct
  misc_feature 1..2383: note = Plasmid PHP30642
  source 1..2383: mol_type = other DNA
ctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccatggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctcggcgtgatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttctagttcgaatgtgagttgatccccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtc

SEQ ID NO: 48 - protein, 31 aa, synthetic construct
  REGION 1..31: note = Consensus sequence motif 1
  VARIANT 10: note = Xaa = His or Asn
  VARIANT 16: note = Xaa = Phe or Tyr
  VARIANT 17: note = Xaa = Val or Ile
  VARIANT 19: note = Xaa = Ser or His
YEKELEEMKXMTRQEXXAXLRRKSSGFSRGA

SEQ ID NO: 49 - protein, 63 aa, synthetic construct
  REGION 1..63: note = Consensus sequence motif 2
  VARIANT 2: note = Xaa = Ile or Met
  VARIANT 36: note = Xaa = Gln or Glu
  VARIANT 45: note = Xaa = Ile or Val
  VARIANT 60: note = Xaa = Asp or Glu
  VARIANT 61: note = Xaa = Met or Ile
  VARIANT 62: note = Xaa = Ser or Asn
SXYRGVTRHHQHGRWQARIGRVAGNKDLYLGTFSTXEEAAEAYDXAAIKFRGLNAVTNFXXXR

SEQ ID NO: 50 - protein, 68 aa, synthetic construct
  REGION 1..68: note = Consensus sequence motif 3
  VARIANT 2: note = Xaa = Ile or Gln
  VARIANT 26: note = Xaa = Arg or Lys
  VARIANT 33: note = Xaa = Val or Gly
  VARIANT 34: note = Xaa = Tyr or Arg
  VARIANT 35: note = Xaa = Leu or Gln
  VARIANT 42: note = Xaa = Glu or Asp
  VARIANT 58: note = Xaa = Pro or Thr
  VARIANT 61: note = Xaa = Thr or His
  VARIANT 62: note = Xaa = Thr or Ile
  VARIANT 66: note = Xaa = Ile, Val, or Leu
SXYRGVTRHRWTGRYEAHLWDNSCRXEGQXRKXXXGGYDKEXKAARAYDLAALKYWGXXTXXNFPXSN

SEQ ID NO: 51 - protein, 8 aa, synthetic construct
  REGION 1..8: note = Consensus sequence motif 4
  VARIANT 3: note = Xaa = Leu or Val
  VARIANT 4: note = Xaa = Glu or Ala
  VARIANT 5: note = Xaa = Asp or Asn
PKXXXFLG

SEQ ID NO: 52 - protein, 13 aa, synthetic construct
  REGION 1..13: note = Consensus sequence motif 5
  VARIANT 6: note = Xaa = Ile or Val
  VARIANT 9: note = Xaa = Ala or Leu
  VARIANT 13: note = Xaa = Leu or Arg
SSTLPXGGXAXXX

SEQ ID NO: 53 - protein, 9 aa, synthetic construct
  REGION 1..9: note = Consensus sequence motif 6
  VARIANT 4: note = Xaa = Gly or Ser
NWLXFSLSP

SEQ ID NO: 54 - protein, 10 aa, synthetic construct
  REGION 1..10: note = Consensus sequence motif 7
  VARIANT 1: note = Xaa = Gly or Glu
  VARIANT 7: note = Xaa = Thr or Asn
XLSMIKXWLR

SEQ ID NO: 55 - protein, 8 aa, synthetic construct
  REGION 1..8: note = Consensus sequence motif 8
PXFXXWND

SEQ ID NO: 56 - protein, 5 aa, synthetic construct
  REGION 1..5: note = Consensus sequence motif 9
  VARIANT 2: note = Xaa = Ser, Thr, or Ala
LXLSM

SEQ ID NO: 57 - protein, 7 aa, synthetic construct
  REGION 1..7: note = Consensus sequence motif 10
  VARIANT 4: note = Xaa = Gln or Pro
WCKXEQD

SEQ ID NO: 58 - protein, 7 aa, synthetic construct
  REGION 1..7: note = Consensus sequence motif 14
WPTIAFQ

SEQ ID NO: 59 - protein, 11 aa, synthetic construct
  REGION 1..11: note = Consensus sequence motif 15
  VARIANT 2: note = Xaa = Ser or Thr
SXGSNSVVYNG

SEQ ID NO: 60 - protein, 7 aa, synthetic construct
  REGION 1..7: note = Consensus sequence motif 19
  VARIANT 4: note = Xaa = Ser or Asn
QDWXMRG

SEQ ID NO: 61 - DNA, 975 bp, Zea mays
  source 1..975: mol_type = genomic DNA
  CDS 1..975
atggagacgccacagcagcaatccgccgccgccgccgccgccgccgcccacgggcaggacgacggcgggtcgccgccgatgtcgccggcctccgccgcggcggcggcgctggcgaacgcgcggtggaacccgaccaaggagcaggtggccgtgctggaggggctgtacgagcacggcctgcgcacccccagcgcggagcagatacagcagatcacgggcaggctgcgggagcacggcgccatcgagggcaagaacgtcttctactggttccagaaccacaaggcccgccagcgccagaggcagaagcaggacagcttcgcctacttcagcaggctcctccgccggcccccgccgctgcccgtgctctccatgccccccgcgccaccgtaccatcacgcccgcgtcccggcgccgcccgcgataccgatgccgatggcgccgccgccgcccgctgcatgcaacgacaacggcggcgcgcgtgtgatctacaggaacccattctacgtggctgcgccgcaggcgccccctgcaaatgccgcctactactacccacagccacagcagcagcagcagcagcaggtgacagtcatgtaccagtacccgagaatggaggtagccggccaggacaagatgatgaccagggccgcggcgcaccagcagcagcagcacaacggcgccgggcaacaaccgggacgcgccggccaccccagccgcgagacgctccagctgttcccgctccagcccaccttcgtgctgcggcacgacaaggggcgcgccgccaacggcagtaataacgactccctgacgtcgacgtcgacggcgactgcgacagcgacagcgacagcgacagcgtccgcttccatctccgaggactcggatggcctggagagcggcagctccggcaagggcgtcgaggaggcgcccgcgctgccgttctatgacttcttcgggctccagtcctccggaggccgctga

SEQ ID NO: 62 - protein, 324 aa, Zea mays
METPQQQSAAAAAAAAHGQDDGGSPPMSPASAAAAALANARWNPTKEQVAVLEGLYEHGLRTPSAEQIQQITGRLREHGAIEGKNVFYWFQNHKARQRQRQKQDSFAYFSRLLRRPPPLPVLSMPPAPPYHHARVPAPPAIPMPMAPPPPAACNDNGGARVIYRNPFYVAAPQAPPANAAYYYPQPQQQQQQQVTVMYQYPRMEVAGQDKMMTRAAAHQQQQHNGAGQQPGRAGHPSRETLQLFPLQPTFVLRHDKGRAANGSNNDSLTSTSTATATATATATASASISEDSDGLESGSSGKGVEEAPALPFYDFFGLQSSGGR

SEQ ID NO: 63 - DNA, 909 bp, Zea mays
  source 1..909: mol_type = genomic DNA
  CDS 1..909
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtga

SEQ ID NO: 64 - protein, 302 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGFYAAGNGGGSAVLLDTSSDWGSSGAAMATETCFLQDYMGVTDTGSSSQWPRFSSSDTIMAAAAARAATTRAPETLPLFPTCGDDGGSGSSSYLPFWGAASTTAGATSSVAIQQQHQLQEQYSFYSNSNSTQLAGTGNQDVSATAAAAAALELSLSSWCSPYPAAGSM

SEQ ID NO: 65 - DNA, 961 bp, Zea mays
  source 1..961: mol_type = genomic DNA
gatccgattgactatctcattcctccaaacccaaacacctcaaatatatctgctatcgggattggcattcctgtatccctacgcccgtgtaccccctgtttagagaacctcccaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacccaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggaagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctcaagcatcagcggcagcggcagcggcaggagctctgggcagcgtgcgcacgtggggtacctagctcgctctgctagcctaccttaa

SEQ ID NO: 66 - DNA, 1917 bp, Zea mays
  source 1..1917: mol_type = genomic DNA
  CDS 1..1917
atgaccagcaacagcagccagaacatgagcagctgcagcaccggcggaagcgacgcggcggtcggcggcggcagctggctcggcttctcgctgtcgcctcacatggcggcgaccatggacggcgcggccgacggcgttccggtgcagcaccaccaccacgaaggcctcttctaccctcccgtcgtcagctcctcgcccgcgcccttctgctacgctctcggcggcggccaagatggcctcgccacggcggccgccaatggtggcggggggttctaccccgggctctcctctatgccgctcaagtccgacggctccctatgcatcctggaggccctccacaggagcgagcaagaacggcacggggtggtggtgtcgtcgtcgtcgcccaaactggaggatttcttgggcgcgagcgcgagcacggcgatggcgctgagcttggacagctccagcttctactacggctgcggccacggccacggccacgaccaaggcgggtacctgcagccaatgcagtgcgcggtgatgcccggctcgggcgggcacgacgtgtacggcggcgggcacgcgcagatggtggacgagcagtccgccgcggcaatggcggcgagctggttctccgcccgcggcaatggcggctacgacgtcgacggcgccggcgccggcgccatcgtgccgttgcagggccacccgcacccgctcgccctctccatgagctccgggacggggtcccagtccagcagcgtcaccatgcaagtcggcagcgcccacgccgacgccgtcaccgagtacatcgccatggacgggagcaagaagcgcggcgccggcaacggcgctagtgccgggcagaagcagcccaccatccaccgcaagaccatcgacacattcgggcagcgcacgtcgcagtaccgcggcgtcaccaggcataggtggacggggaggtatgaggcgcacctctgggacaacagctgcaggaaggaagggcagacccggaaaggccggcaagtttatctcggcgggtatgacgtggaggagaaggccgcgagggcatatgacctggcggcgctcaagtactgggggacgtccacgcacgtgaatttcccggtggaggactacagggaagagctggaggagatgaagaacatgaccagacaggagtacgtcgctcacctgagaaggaaaagcagcggcttctcgcgcggcgcttcgatctaccggggagtcaccaggcatcaccagcacgggcggtggcaggcgcgcatcggccgcgtctcgggcaacaaggacctctacctcggaacgttcagcacccaggaggaggcggcggaggcgtacgacgtggccgcgatcaagttccgcggcctcagcgcggtcaccaacttcgacatcacgcggtacgacgtggacaagatcatggagagcagcacgctgctcccgggcgagcaggtccggcgcaggaaggaaggcgccgacgccgcggtctcggaggccgccgccgcgctggtgcaggccggcaactgcatgacggacacctggaagatccaggcggcgctgccggctgccgcgcgggccgacgagcgcggcgccggccagcagcagcgtcaggacttgctgtcgagcgaggccttctcgctgctccacgacatcgtgtccgtcgacgccgctgctggtacagggacagggggcatgtcgaacgcgtcctcgtcgctggcccccagcgtgagcaactcccgggagcagagcccggaccggggcggcgccagcctcgccatgctcttcgccaagcccgccgcggcgcccaagctggcttgcccgctgccgctggggtcctgggtgtcgccgtccgcggtgtccgccaggccgcccggcgtgtcaatcgcgcacctgccggtgttcgccgcgtggaccgacgcatga

SEQ ID NO: 67 - protein, 638 aa, Zea mays
MTSNSSQNMSSCSTGGSDAAVGGGSWLGFSLSPHMAATMDGAADGVPVQHHHHEGLFYPPVVSSSPAPFCYALGGGQDGLATAAANGGGGFYPGLSSMPLKSDGSLCILEALHRSEQERHGVVVSSSSPKLEDFLGASASTAMALSLDSSSFYYGCGHGHGHDQGGYLQPMQCAVMPGSGGHDVYGGGHAQMVDEQSAAAMAASWFSARGNGGYDVDGAGAGAIVPLQGHPHPLALSMSSGTGSQSSSVTMQVGSAHADAVTEYIAMDGSKKRGAGNGASAGQKQPTIHRKTIDTFGQRTSQYRGVTRHRWTGRYEAHLWDNSCRKEGQTRKGRQVYLGGYDVEEKAARAYDLAALKYWGTSTHVNFPVEDYREELEEMKNMTRQEYVAHLRRKSSGFSRGASIYRGVTRHHQHGRWQARIGRVSGNKDLYLGTFSTQEEAAEAYDVAAIKFRGLSAVTNFDITRYDVDKIMESSTLLPGEQVRRRKEGADAAVSEAAAALVQAGNCMTDTWKIQAALPAAARADERGAGQQQRQDLLSSEAFSLLHDIVSVDAAAGTGTGGMSNASSSLAPSVSNSREQSPDRGGASLAMLFAKPAAAPKLACPLPLGSWVSPSAVSARPPGVSIAHLPVFAAWTDA

SEQ ID NO: 68 - DNA, 2260 bp, Zea mays
  source 1..2260: mol_type = genomic DNA
cttccctaacctttgcactgtccaaaatggcttcctgatcccctcacttcctcgaatcaatctaagaagaaactcaagccgcaaccattaggggcagattaattgctgcactttcagataatcaaccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaa

SEQ ID NO: 69 - DNA, 3766 bp, Sorghum bicolor
  source 1..3766: mol_type = genomic DNA
atggctactgtgaacaactggctcgctttctccctctccccgcaggagctgccgcccacccagacggactccaccctcatctctgccgccaccaccgacgatgtctccggcgatgtctgcttcaacatcccccaaggtatgcatctatcgatcgatatatgtacgtacagtgcgcatatatatatatatctgcagtttgtggtacgaatactgattgaagctagcatgaaatgtcgtttgttctttcagattggagcatgaggggatccgagctttcggcgctcgtcgccgagccgaagctggaggacttcctcggcggaatctccttctccgagcagcaccacaaggccaactgcaacatgatccccagcactagcagcacagcttgctacgcgagctcgggtgctaccgccggctaccatcaccagctgtaccaccagcccaccagctccgcgctccacttcgctgactccgtcatggtggcctcctcggccggcggcgtccacgacggaggtgccatgctcagcgcggccagcgctaatggtagcgctggcgctggcgctgccagtgccaatggcagcggcagcatcgggctgtccatgatcaagaactggctgcggagccaaccagctcccatgcagccgagggtggcggcggctgagagcgtgcaggggctctctttgtccatgaacatggcgggggcgacgcaaggcgccgctggcatgccacttcttgctggagagcgcggccgggcgcccgagagtgtctcgacgtcggcacagggtggagccgtcgtcacggctccaaaggaggatagcggtggcagcggtgttgccgccaccggcgccctagtagccgtgagcacggacacgggtggcagcggcgcgtcggctgacaacacggcaaggaagacggtggacacgttcgggcagcgcacgtcgatttaccgtggcgtgacaaggtaataagggtccggtattacaatgaatcgtcacttcgtcagagaactaaactagcacaaatcagcaatgaatcaagtaatatcatgaaatttagaaaagccgttagcaatgcaaggagctatcattatagatttgattgcatctagacagttctgaattaaatgagtagggcaatgtgtagcctttgatgatctcgctgattattaggagtgccatttgtattggctatgattgtggtatatacagcagtagacaattaacaaaaggctaccactttcgaattattttaggcatagatggactgggagatatgaagcacatctgtgggacaacagttgcagaagggaaggacaaactcgcaagggtcgtcaaggtaccaatataatgcaatacaccgtatttaaatatatatgcttttctgtaattaagtttatactttcacaaaactgacattacttcgcattatcatttttggattgtcgtcgtcatgattggcgggattgaaatgaactattgaatctacagtctatttaggtaagcgatttcacttggttattaatttgggaccaactacttaatccagtttgtttttcccctataaccattattttttcatctgtgttctcaactcttacttttccatcttgttccactgataggtggctatgataaagaggagaaagctgctagggcttatgatctggctgctcttaagtactggggtcccacgacaacaacaaattttccagtatgtatatgtagaatgcagttttacttcactgaagatcatacctttgctatgtctcaaatgccgttcattagttagtggatctgaagtgaaggttctgtaatttttgttaactatgtacattgctggaattgtacttaaagtcatttgtttttgtatatctaggtgaataactacgaaaaggagctggaggatatgaagcacatgacaaggcaggagtttgtagcgtctctgagaaggtcggtcgaacagcattgattaatcaatgccaactctattgaataaacatctactctgttaattgttaaagtttgagagaaagatctgcatgttagatcttaatagaccactgtatatgaatgcaggaagagcagtggtttctccagaggtgcatccatttacaggggagtgactaggtatgaattcatataatggcgtcaacaaacacacatacactttgattgaggaggcgaatgcacgcatggattgaatgtgaatggtgttttacttgaactatgtaattataggcatcaccagcatggaagatggcaagcacggattggacgagttgcagggaacaaggatctctacttgggcaccttcagtaagtatcagagatgttttctcattgtatatagaggagtacttctatatgtatatatacattcagttattcaccacacaaaagcaaattgcagtcaactaataacaatctcaacgcaatgagaagcaagtgttacagctgatagtacacatttgtagaccttctgcatatggatgttatatatgatgactattaaaaatgtgaccattgcatcaagtcatgcaaagttgcattgcagtagtacatacattacttagtgcatgctcctcaagtggctttttcaaacctgatcccatgtctggcgctattgttgtctcccattcacccgtgcatcaggtcaaaatagtactatgcctcaataagaaacacatgagcatgcactggcagcagcagactaatcaagttctatcatttactaataaactaattaggctacagcatccaaaagattctacccattaagccacaactgttcatgcatgcattcataaaccaggataccaccatgcatgcgtgcaccgtgttcgtgcttggaatattgagctgagccgagtgcacccttgcgtggatgcaggcacgcaggaggaggcagcggaggcatacgacattgcggcgatcaagttccgcggcctcaacgccgtcacaaacttcgacatgagccgctacgacgtcaagagcatcctggacagcagtgcgctccccatcggcagcgccgccaagcgtctcaaggaggccgaggccgccgcgtccgcacagcaccatgccggcgtggtgagctacgacgtcggccgcatagcctcacagctcggcgacggcggcgccctggcggcggcgtacggcgcgcactaccatggcgcctggccgaccatcgcgttccagccgagcgcggccacgggcctgtaccacccgtacgcgcagccgatgcgcgggtggtgcaagcaggagcaggaccacgcggtgatcgcggccgcgcacagcctgcaggagctccaccacctgaacctgggtgctgccgccggcgcgcacgacttcttctcggcggggcagcaggcggcgatgcacggcctgggtagcatggacaatgcatcactcgagcacagcaccggctccaactccgtcgtgtacaacggtgttggtgatagcaacggcagcaccgtcgtcggcagtggtggctacatgatgcctatgagcgctgccacggcgacggctaccacggcaatggtgagccacgagcaggtgcatgcacgggcacagggtgatcaccacgacgaagccaagcaggctgctcagatggggtacgagagctacctggtgaacgcagagaactatggcggcgggaggatgtctgcggcctgggcgactgtctcagcgccaccggcggcaagcagcaacgataacatggcggacgtcggccatggcggcgcacagctcttcagtgtctggaacgatact

SEQ ID NO: 70 - protein, 530 aa, Glycine max
MDSSSSSPPNSTNNNSLAFSLSNHFPNPSSSPLSLFHSFTYPSLSLTGSNTVDAPPEPTAGAGPTNLSIFTGGPKFEDFLGGSAATATTVACAPPQLPQFSTDNNNHLYDSELKSTIAACFPRALAAEQSTEPQKPSPKKTVDTFGQRTSIYRGVTRHRWTGRYEAHLWDNSCRREGQSRKGRQVYLGGYDKEDKAARAYDLAALKYWGPTTTTNFPISNYEKELEEMKNMTRQEFVASLRRKSSGFSRGASIYRGVTRHHQHGRWQARIGRVAGNKDLYLGTFSTQEEAAEAYDIAAIKFRGLNAVTNFDMSRYDVKSIANSTLPIGGLSGKNKNSTDSASESKSHEPSQSDGDPSSASSVTFASQQQPSSSNLSFAIPIKQDPSDYWSILGYHNTPLDNSGIRNTTSTVTTTTFPSSNNGTASSLTPFNMEFSSAPSSTGSDNNAAFFSGGGIFVQQQTSHGHGNASSGSSSSSLSCSIPFATPIFSLNSNTSYESSAGYGNWIGPTLHTFQSHAKPSLFQTPIFGME

SEQ ID NO: 71 - protein, 528 aa, Glycine max
MDSCSSPPNNNSLAFSLSNHFPNPSSSPLSLFHSFTYPSLSLTGSHTADAPPEPIAGGGATNLSIFTGAPKFEDFLGGSSATATATTCAPPQLPQFSTDNNNHLYDSELKTTIAACFPRAFAAEPTTEPQKPSPKKTVDTFGQRTSIYRGVTRHRWTGRYEAHLWDNSCRREGQSRKGRQVYLGGYDKEDKAARAYDLAALKYWGPTTTTNFPISNYEKELEEMKNMTRQEFVASLRRKSSGFSRGASIYRGVTRHHQHGRWQARIGRVAGNKDLYLGTFSTQEEAAEAYDIAAIKFRGLNAVTNFDMSRYDVKSIANSTLPIGGLSGKNKNSTDSASESKSHEASRSDERDPSAASSVTFASQQQPSSSTLSFAIPIKQDPSDYWSILGYHNSPLDNTGIRNTTSVTATSFPSSNNGTTSSLTPFHMEFSNAPTSTGSDNDAAFFSGGGIFVQQQSGHGNGHGSGSSGSSSSSLSCSIPFATPIFSLNSNTSYENSAGYGNWIGPTLHTFQSHAKPSLFQTPIFGME

SEQ ID NO: 72 - protein, 488 aa, Zea mays
MDMDMSSAYPHHWLSFSLSNNYHHGLLEAFSNSSGTPLGDEQGAVEESPRTVEDFLGGVGGAGAPPQPAAAADQDHQLVCGELGSITARFLRHYPAAPAGTTVENPGAVTVAAMSSTDVAGAESDQARRPAETFGQRTSIYRGVTRHRWTGRYEAHLWDNSCRREGQSRKGRQVYLGGYDKEEKAARAYDLAALKYWGPTTTTNFPVSNYEKELEEMKSMTRQEFIASLRRKSSGFSRGASIYRGVTRHHQHGRWQARIGRVAGNKDLYLGTFSTQEEAAEAYDIAAIKFRGLNAVTNFDMSRYDVESILSSDLPVGGGASGRAPAKFPLDSLQPGSAAAMMLAGAAAASQATMPPSEKDYWSLLALHYQQQQEQERQFPASAYEAYGSGGVNVDFTMGTSSGNNNNNTGSGVMWGATTGAVVVGQQDSSGKQGNGYASNIPYAAAAMVSGSAGYEGSTGDNGTWVTTTTSSNTGTAPHYYNYLFGME

SEQ ID NO: 73 - protein, 495 aa, Oryza sativa
MDMDTSHHYPWLNFSLAHHCEMEEEERGAAAELAAIAGAAPPPKLEDFLGGGCNGGSSGGACPPVQTTAPTAAELYESELKFLAAGFQLSGAAGAAPPVPALLPAAALEQTDETKQLALPPQAAVAPPPEQKKAVDSFGQRTSIYRGVTRHRWTGRYEAHLWDNSCRREGQSRKGRQVYLGGYDKEEKAARAYDLAALKYWGPSTTTNFPVAEYEKELEEMKHMTRQEFVASLRRKSSGFSRGASIYRGVTRHHQHGRWQARIGRVAGNKDLYLGTFGTEEEAAEAYDIAAIKFRGLNAVTNFEIGRYNVESIISSNLPIGSMAGNRSTKAGLELAPSSSADAIAATEANHTGVAPPSTLAFTALPMKYDQADYLSYLALQHHQQGNLQGLGFGLYSSGVNLDFANANGNGAMSNCYTNVSLHEQQQQHQHQHQQEQQQDQQDDQSQSSNNSCGSIPFATPIAFSGSYESSMTAAGTFGYYPNVAAFQTPIFGME

SEQ ID NO: 74 - protein, 558 aa, Arabidopsis thaliana
MKNNNNKSSSSSSYDSSLSPSSSSSSHQNWLSFSLSNNNNNFNSSSNPNLTSSTSDHHHPHPSHLSLFQAFSTSPVERQDGSPGVSPSDATAVLSVYPGGPKLENFLGGGASTTTTRPMQQVQSLGGVVFSSDLQPPLHPPSAAEIYDSELKSIAASFLGNYSGGHSSEVSSVHKQQPNPLAVSEASPTPKKNVESFGQRTSIYRGVTRHRWTGRYEAHLWDNSCRREGQSRKGRQVYLGGYDKEDKAARAYDLAALKYWGPTTTTNFPISNYESELEEMKHMTRQEFVASLRRKSSGFSRGASMYRGVTRHHQHGRWQARIGRVAGNKDLYLGTFSTQEEAAEAYDIAAIKFRGLNAVTNFDISRYDVKSIASCNLPVGGLMPKPSPATAAADKTVDLSPSDSPSLTTPSLTFNVATPVNDHGGTFYHTGIPIKPDPADHYWSNIFGFQANPKAEMRPLANFGSDLHNPSPGYAIMPVMQEGENNFGGSFVGSDGYNNHSAASNPVSAIPLSSTTTMSNGNEGYGGNINWINNNISSSYQTAKSNLSVLHTPVFGLE

SEQ ID NO: 75 - protein, 568 aa, Arabidopsis thaliana
MNSNNWLAFPLSPTHSSLPPHIHSSQNSHFNLGLVNDNIDNPFQNQGWNMINPHGGGGEGGEVPKVADFLGVSKSGDHHTDHNLVPYNDIHQTNASDYYFQTNSLLPTVVTCASNAPNNYELQESAHNLQSLTLSMGSTGAAAAEVATVKASPAETSADNSSSTTNTSGGAIVEATPRRTLETFGQRTSIYRGVTRHRWTGRYEAHLWDNSCRREGQSRKGRQVYLGGYDKEEKAARAYDLAALKYWGPSTTTNFPITNYEKEVEEMKNMTRQEFVASIRRKSSGFSRGASMYRGVTRHHQHGRWQARIGRVAGNKDLYLGTFSTEEEAAEAYDIAAIKFRGLNAVTNFEINRYDVKAILESNTLPIGGGAAKRLKEAQALESSRKREEMIALGSNFHQYGAASGSSSVASSSRLQLQPYPLSIQQPFEHLHHHQPLLTLQNNNDISQYHDSFSYIQTQLHLHQQQTNNYLQSSSHTSQLYNAYLQSNPGLLHGFVSDNNNTSGFLGNNGIGIGSSSTVGSSAEEEFPAVKVDYDMPPSGGATGYGGWNSGESAQGSNPGGVFTMWNE

SEQ ID NO: 76 - protein, 474 aa, Sorghum bicolor
MDMDMSSAYPHHWLSFSLSNNYHHGLLEAFSNSSSAAPLGDEQGTVEESPKMVEDFLGGVGGAGAPPAAATAAEDHQLVCGELGSITAGFLRHYPAPGTTVENPGAVTVAAMSTDVAESDQARRPAETFGQRTSIYRGVTRHRWTGRYEAHLWDNSCRREGQSRKGRQVYLGGYDKEEKAARAYDLAALKYWGATTTTNFPVSNYEKELEEMKSMTRQEFIASLRRKSSGFSRGASIYRGVTRHHQHGRWQARIGRVAGNKDLYLGTFSTQEEAAEAYDIAAIKFRGLNAVTNFDMSRYDVDSILNSDLPVGGGAAGRASKFPLDSLQPGSAAAMIAGAASQAMPPSEKDYWSLLALHYQQQQQQQQFPASAYEAYGSGVNVDFTMGTSSHSSSNTGSGVMWGTTTGAMGQQDSSSSKQGNGYASNIPYAAAAAAMVSGSAGYEGSTGNNGTWVTSSTSTSTAPQYYNYLFGME

SEQ ID NO: 77 - protein, 549 aa, Oryza sativa
MDMNSGWLGFSLSSSSARGYGDGCGEGNGGGDGDGSCSSPVAASPLVAMPLHSDGSVHYDAPDWRHAEAKDPKLEDFMSVSYSNKSSSNLYGSSSSSSCGHADQIKYHHVHDVQAFSTPYFYGHGGSGVGIDINMNAPPAGCTGVLPDHRPPPPQQDHIFLPPHGQYFLGPPNPMAPAPMYNAGGGGGGVVDGSMSISGIKSWLRQAMYVPERSAAALSLSVPAAPPSEAPLPPAAMPVVRKPAQTFGQRTSQFRGVTRHRWTGRYEAHLWDNTCRKEGQTRKGRQVYLGGYDKEEKAARAYDLAALKYWGPTTHINFPLSTYEKELEEMKHMTRQEFIAHLRRNSSGFSRGASMYRGVTRHHQHGRWQARIGRVAGNKDLYLGTFSTQEEAAEAYDIAAIKFRGLNAVTNFDISKYDVKRICSSTHLIGGDLACRRSPTRMLPPDAPAGAAGVDVVVAPGDHQQISAGGGGASDNSDTASDGHRGAHLLHGLQYAHAMKFEAGESSGGGGGDGATTNWMAAAAAAARPVAGIPTTVHHQLPVFALWND

SEQ ID NO: 78 - protein, 553 aa, Glycine max
MNNNWLSFPLSPTHSSLPAHDLQATQYHQFSLGLVNENMDNPFQNHDWNLINTHSSNEIPKVADFLGVSKSENQSDLAALNEIHSNDSDYLFTNNSLVPMQNPVLDTPSNEYQENANSNLQSLTLSMGSGKDSTCETSGENSTNTTVEVAPRRTLDTFGQRTSIYRGVTRHRWTGRYEAHLWDNSCRREGQSRKGRQVYLGGYDKEEKAARAYDLAALKYWGTSTTTNFPISNYEKELDEMKHMTRQEFVAAIRRKSSGFSRGASMYRGVTRHHQHGRWQARIGRVAGNKDLYLGTFSTEEEAAEAYDIAAIKFRGLNAVTNFDMSRYDVKAILESNTLPIGGGAAKRLKEAQALESSRKREEMIALGSSSTFQYGTSASSSRLHAYPLMQHHHQFEQPQPLLTLQNHDISSSHFSHQQDPLHHQGYIQTQLQLHQQSGASSYSFQNNAQFYNGYLQNHPALLQGMMNMGSSSSSSSVLENNNSNNNNNNVGGFVGSGFGMASNATAGNTVGTAEELGLVKVDYDMPAGGYGGWSAADSMQTSNGGVFTMWND

SEQ ID NO: 79 - protein, 509 aa, Medicago truncatula
MDKSSSSPPTNTNNTSLAFSLSNNNFPNPSHSSSSHLSLFHSFTPYPSSIIPPSLTLTGSNNPVEASPEATDGGTTNLSIFTGGHKFEDFLGSSVAPTRTAAATCAPTQLQQFSTDNDVYNSELKKTIAACFPGGYPTEPNSEPQKPSPKKTVDTFGQRTSIYRGVTRHRWTGRYEAHLWDNSCRREGQSRKGRQGGYDKEEKAARAYDLAALKYWGPTTTTNFPISNYEKEIDDMKNMTRQEFVASLRRKSSGFSRGASIYRGVTRHHQHGRWQARIGRVAGNKDLYLGTFSTQEEAAEAYDIAAIKFRGLNAVTNFDMSRYDVKSIANCSLPIGGLSNKNNKNSTDCVSETKINEPIQSDEIDHPSSTSSATTLSFALPIKQDPSTDYWSNILGFHNNPSAVTTTTIPFNMDFSAHVPSNTNSDNPHNAAFFSGSGIFVQQQNMNGSSGSNSSSSSSASTSSIPFATPIFSLNSNSSSYGNGNNWIGHTFQTHAKPSLFQTPIFGME

SEQ ID NO: 80 - protein, 492 aa, Zea mays
MDTSHHYHPWLNFSLAHHCDLEEEERGAAAELAAIAGAAPPPKLEDFLGGGVATGGPEAVAPAEMYDSDLKFIAAAGFLGGSAAAAATSPLSSLDQAGSKLALPAAAAAPAPEQRKAVDSFGQRTSIYRGVTRHRWTGRYEAHLWDNSCRREGQSRKGRQVYLGGYDKEEKAARAYDLAALKYWGSSTTTNFPVAEYEKEVEEMKNMTRQEFVASLRRKSSGFSRGASIYRGVTRHHQHGRWQARIGRVAGNKDLYLGTFSTEEEAAEAYDIAAIKFRGLNAVTNFEISRYNVETIMSSNLPVASMSSSAAAAAGGRSSKALESPPSGSLDGGGGMPVVEASTAPPLFIPVKYDQQQQEYLSMLALQQHHQQQQAGNLLQGPLVGFGGLYSSGVNLDFANSHGTAAPSSMAHHCYANGTASASHEHQHQMQQGGENETQPQPQQSSSSCSSLPFATPVAFNGSYESSITAAGPFGYSYPNVAAFQTPIYGME

SEQ ID NO: 81 - protein, 469 aa, Oryza sativa
MDMDMSSAYPHHWLSFSLSNNYHHGLLEALSTTSAPPLGEEGPAEGAPKMEDFLGGLGGGGGAVAAAPAAAPEDQLSCGELGSIAAGFLRRYPAPENAGGVTIAMATDAAAELADPARRTAETFGQRTSIYRGVTRHRWTGRYEAHLWDNSCRREGQSRKGRQVYLGGYDKEEKAARAYDLAALKYWGPTTTTNFPVANYETELEEMKSMTRQEFIASLRRKSSGFSRGASIYRGVTRHHQHGRWQARIGRVAGNKDLYLGTFSTQEEAAEAYDIAAIKFRGLNAVTNFDMSRYDVDSILNSDLPVGGGAATRASKFPSDPSLPLPSPAMPPSEKDYWSLLALHYHHHQQQQQQQQFPASAFDTYGCSSGVNVDFTMGTSSHSGSNSNSSSSSAIWGTAAGAAMGRQQNGGSSNKQSNSYSGNNIPYAAAAAMTSGSALYGGSTGSNGTWVASNTSTAPHFYNYLFGME

SEQ ID NO: 82 - protein, 562 aa, Glycine max
MNNNWLSFPLSPTHSSLPAHDLQATQYHQFSLGLVNENMENPFQNHDWSLINTHSSSEVPKVADFLGVSKSENESDLAASLNEIQSNDSDYLFTNNSLVPMQNPAVDTPSNEYQENANSSLQSLTLSMGSGKDSTCETSGDNSTNTTTTTTVEAAPRRTLDTFGQRTSIYRGVTRHRWTGRYEAHLWDNSCRREGQSRKGRQVYLGGYDKEEKAARSYDLAALKYWGTSTTTNFPISNYEKELDEMKHMTRQEFVAAIRRKSSGFSRGASMYRGVTRHHQHGRWQARIGRVAGNKDLYLGTFSTEEEAAEAYDIAAIKFRGLNAVTNFDMSRYDVKAILESNTLPIGGGAAKRLKEAQALESSRKREEMIALGSSTFQYGTTSSNSRLHAYPLMQHHHQFEQPQPLLTLQNHDISSHFSHQQDPLHQGYIQTQLQLHQQQSGGSSSYSFQNNNINNAQFYNGYNLQNHPALLQGMINMGSSSSSSVLENNNSNNNNVGGFVGSGFGMASNATSGNTVGTAEELGLVKVDYDMPTGGYGGWSAAAAAESMQTSNSGVFTMWND

SEQ ID NO: 83 - protein, 574 aa, Arabidopsis thaliana
MNSNNWLGFPLSPNNSSLPPHEYNLGLVSDHMDNPFQTQEWNMINPHGGGGDEGGEVPKVADFLGVSKPDENQSNHLVAYNDSDYYFHTNSLMPSVQSNDVVVAACDSNTPNNSSYHELQESAHNLQSLTLSMGTTAGNNVVDKASPSETTGDNASGGALAVVETATPRRALDTFGQRTSIYRGVTRHRWTGRYEAHLWDNSCRREGQSRKGRQVYLGGYDKEDKAARSYDLAALKYWGPSTTTNFPITNYEKEVEEMKHMTRQEFVAAIRRKSSGFSRGASMYRGVTRHHQHGRWQARIGRVAGNKDLYLGTFSTEEEAAEAYDIAAIKFRGLNAVTNFEINRYDVKAILESSTLPIGGGAAKRLKEAQALESSRKREAEMIALGSSFQYGGGSSTGSGSTSSRLQLQPYPLSIQQPLEPFLSLQNNDISHYNNNNAHDSSSFNHHSYIQTQLHLHQQTNNYLQQQSSQNSQQLYNAYLHSNPALLHGLVSTSIVDNNNNNGGSSGSYNTAAFLGNHGIGIGSSSTVGSTEEFPTVKTDYDMPSSDGTGGYSGWTSESVQGSNPGGVFTMWNE

SEQ ID NO: 84 - protein, 543 aa, Medicago truncatula
MNNNWLSFPLSPSHSSLPSNDLQATQYHHFPLGLVNDNMENPFQNHDWNLMNTHNSNEVPKVADFLGVCKSENHSDLATPNEIQSNDSDYLFTNNNTLMPMQNQMVTTCTNEYQEKASNSNLQSLTLSMGSGKDSTCETSGENSTNTVEVAVPKRTSETFGQRTSIYRGVTKHRWTGRYEAHLWDNSCRREGQSRKGRQGGYDKEEKAARSYDLAALKYWGTSTTTNFPVSNYEKEIDEMKHMTRQEFVASIRRKSSGFSRGASMYRGVTRHHQHGRWQARIGRVAGNKDLYLGTFSTEEEAAEAYDIAAIKFRGLNAVTNFDMTRYDVKAILESNTLPIGGGAAKRLKEAQALETSRKREEMLALNSSSFQYGTSSSSNTRLQPYPLMQYHHQFEQPQPLLTLQNNHESLNSQQFSQHQGGGYFQTQLELCQQQNQQPSQNSNIGSFYNGYYQNHPGLFQMNNIGSSSSSSVMGNNGGGSSGIYSNSGGLISNNAVEEFVPVKVDYDMQGDGSGFGGWSAAGENMQTADLFTMWNDYETREN

SEQ ID NO: 85 - protein, 543 aa, Zea mays
MDMNNGWLGFSLSPSAASRGGYGYGDGGGGASASACGDGEGSCPSPAAAASPLPLVAMPLDDSLHYSSAPDWRHGAAEAKGPKLEDFMSITCSNKSSGRSLYDSCGHHDDEQASKYHEVHGIHPLSCGSYYHGCISSGGGGGGGIGLGINMNAPPCTGGFPDHQHHQFVPSSHHGQYFLGAPAASAGPPAGAAMPMYNAGGGSVVGGSMSISGIKSWLREAMYVPPERPAAAALSLAVTDDVPPAEPPQLLPAPLPVHRKPAQTFGQRTSQFRGVTRHRWTGRYEAHLWDNTCRKEGQTRKGRQVYLGGYDREEKAARAYDLAALKYWGPSTHINFPLSHYEKELEEMKHMSRQEFIAHLRRNSSGFSRGASMYRGVTRHHQHGRWQARIGRVAGNKDLYLGTFSTQEEAAEAYDIAAIKFRGLNAVTNFDISKYDVKRICASTHLIGGGDACRRSPTRPPDAAPALAGGADRSSDAPGDQAASDNSDTSDGHRGAHLLHGLQYGHPMKLEAGEGSSWMAAAAAARPVPGVHQLPMFALWNDC

SEQ ID NO: 86 - protein, 512 aa, Glycine max
MSNWLGFSLTPHLRIDEEFERENQERGGGIILFEKKKTKWRYDSAIGGGNSNEEGPKLEDFLGCYSNSPAKVFCQDSQPDQNQSQNNVSKINIETGDNLTNPSSLLHSFHAYNDNSHALIPTNGMYKSWLAQTQFSSDGKPSNEANGCNFQSLSLTMSPSVQNGVGAISSVQVNEDSRKRVMAKSHAREPVPRKSIDTFGQRTSQYRGVTRHRWTGRYEAHLWDNSCRKEGQTRKGRQGGYDKEEKAAKAYDLAALKYWGPTTHINFPLSTYEKELEEMKHMTRQEFVANLRRKSSGFSRGASVYRGVTRHHQHGRWQARIGRVAGNKDLYLGTFSTQEEAAEAYDIAAIKFRGTSAVTNFDISRYDVKRICSSSTLIAGDLAKRSPKESPAPPPPLAITDGEHSDELSNMMWNANNSDEQAQNESGGAEFNNNVTESSSSQQVSPSSNKDALNPQSPNEFGVSGADYGHGYFTLDGPKYDDGNNENDHMSTNRLGNLGLVNQVPMFALWNE

SEQ ID NO: 87 - protein, 485 aa, Sorghum bicolor
MDTSHHYPWLNFSLAHHGDLEEEERGAAAELAAIAGAAPPPKLEDFLGGGVINGESARSGGGVPVAAPEVSAPAEMYDSDLKFIAAAGFLGGGSAAGPVATSPLSSLDQADPKLALPAAAAAAPAPEQRKAVDSFGQRTSIYRGVTRHRWTGRYEAHLWDNSCRREGQSRKGRQGGYDKEEKAARAYDLAALKYWGSSTTTNFPVAEYEKELEEMKTMTRQEFVASLRRKSSGFSRGASIYRGVTRHHQHGRWQARIGRVAGNKDLYLGTFSTEEEAAEAYDIAAIKFRGLNAVTNFEISRYNVESIMNSNIPMGSMSAGGRSNKALESPPSGSPDAMPVEASTAPLFAALPVKYDQQQQDYLSMLALQHHQQGNLQGLGFGLYSSGVNLDFANSHSTASSMTHCYVNGGTVSSHEQHQHHQQLQDHQQQGESETQQSSNSCSSLPFATPIAFNGSYESSMTAAGPFGYSYPNVAAFQTPIYGME

SEQ ID NO: 88 - protein, 507 aa, Glycine max
MARATNWLSFSLSPMEMLRTSEPQFLQYDAASATSSHHYYLDNLYTNGWGNGSLKFEQNLNHSDVSFVESSSQSVGHVPPPPPKLEDFLGDSSAVMRYSDSQTETQDSSLTHIYDHHHHHHHHHGSTSYFGGDQQDLKAITGFQAFSTNSGSEVDDSASIGKAQASEFGTHSIESSGNEFAAFSGGTTGTLSLAVALSSEKAVVAAESNSSKKIVDTFGQRTSIYRGVTRHRWTGRYEAHLWDNSCRREGQARKGRQGGYDKEEKAARAYDLAALKYWGPTATTNFPVSNYSKEVEEMKHVTKQEFIASLRRKSSGFSRGASIYRGVTRHHQQGRWQARIGRVAGNKDLYLGTFATEEEAAEAYDIAAIKFRGANAVTNFEMNRYDVEAIMKSSLPVGGAAKRLRLSLESEQKAPPVNSSSQQQNPQCGNVSGSINFSAIHQPIASIPCGIPFDSTTAYYPHNLFQHFHPTNAGAAASAVTSANATALTALPASAATEFFIWPHQSY

SEQ ID NO: 89 - protein, 569 aa, Arabidopsis thaliana
MEMLRSSDQSQFVSYDASSAASSSPYLLDNFYGWSNQKPQEFFKEEAQLAAAASMADSTILTTFVDPQSHHSQNHIPKLEDFLGDSSSIVRYSDNSQTDTQDSSLTQIYDPRHHHNQTGFYSDHHDFKTMAGFQSAFSTNSGSEVDDSASIGRTHLAGDYLGHVVESSGPELGFHGGSTGALSLGVNVNNNTNHRNDNDNHYRGNNNGERINNNNNNDNEKTDSEKEKAVVAVETSDCSNKKIADTFGQRTSIYRGVTRHRWTGRYEAHLWDNSCRREGQARKGRQVYLGGYDKEDKAARAYDLAALKYWNATATTNFPITNYSKEVEEMKHMTKQEFIASLRRKSSGFSRGASIYRGVTRHHQQGRWQARIGRVAGNKDLYLGTFATEEEAAEAYDIAAIKFRGINAVTNFEMNRYDVEAIMKSALPIGGAAKRLKLSLEAAASSEQKPILGHHQLHHFQQQQQQQQLQLQSSPNHSSINFALCPNSAVQSQQIIPCGIPFEAAALYHHHQQQQQHQQQQQQQNFFQHFPANAASDSTGSNNNSNVQGTMGLMAPNPAEFFLWPNQSY

SEQ ID NO: 90 - protein, 574 aa, Medicago truncatula
MSNWLGFSLTPHLRIDEEFGTENQNQNQNHVAEGSEIGRNYVTPSSHPHPHHLSIMPLRSDGSLCVSDSFTPQEWRYENAITDGNSNEEGPKLEDFLGCYSNQNQNSTTTSTMSKINVNVSPSFCTNNNPEIDTRENLTNQSLIHSFHAYNDHSNNNHHALIHDNSMYKSWMTQTQFSSEGKTTSSDGNGFQSLNLTMSPCVQNGVGGGVGSAISNVQVNEDPRKRSLSKSNAREPVPRKSIDTFGQRTSQYRGVTRHRWTGRYEAHLWDNSCRKEGQTRKGRQGGYDKEEKAAKAYDLAALKYWGPTTHINFPLSTYDKELEEMKHMTRQEFVANLRRKSSGFSRGASVYRGVTRHHQHGRWQARIGRVAGNKDLYLGTFSTQEEAAEAYDIAAIKFRGTSAVTNFDISRYDVKRICSSSTLITGDLAKRSPKDSTPPATTAEDFNSCGSSSTLSQPPPLTITDGEQHSDELSNMVWNSNNDEQKPQNGTNITESSQHGSPSNKNEMNPQSPKCSLGLPNEFGVSGADYGHGYFTLHGPKFDDGSNENDHMNNNRLGNLGLVNQVPMFALWNE

SEQ ID NO: 91 - protein, 541 aa, Sorghum bicolor
MDMNNGWLGFSLSPSAGRGGYGDGGASASGDGGDGSCSSPAAAASPVPLVAMPLQPDGSLHYTSAPDWRHGAAEANGPKLEDFMSVTCSSNNKRSSSSSSFYDRCSHAEQANKYHEVHDLQPLSCGSYYHGSSGGGGNGIALGINMNAPPCSGGGFPDHHHHHQFVSSHHGQYFLGAPLNASPPGAVPMYSAGGGGVGGSMSISGIKSWLREAMYVPPERPVAAAAALSLAVTDDVGAEPPQLLPAAPMPPVHRKPAQTFGQRTSQFRGVTRHRWTGRYEAHLWDNTCRKEGQTRKGRQGGYDREEKAARAYDLAALKYWGPSTHINFPLSHYEKELEEMKHMSRQEFIAHLRRNSSGFSRGASMYRGVTRHHQHGRWQARIGRVAGNKDLYLGTFSTQEEAAEAYDIAAIKFRGLNAVTNFDISKYDVKRICASTHLIGGGDACRRSPTQPPDAPALAIDAAGADRSSDAPGGGDQAVSDNSDTSAGHRGAHLLHGLQYGHPMKLEAGEGSSWMAAATAAAARPVAGVHQLPVFALWNDC

SEQ ID NO: 92 - protein, 555 aa, Arabidopsis thaliana
MKSFCDNDDNNHSNTTNLLGFSLSSNMMKMGGRGGREAIYSSSTSSAATSSSSVPPQLVVGDNTSNFGVCYGSNPNGGIYSHMSVMPLRSDGSLCLMEALNRSSHSNHHQDSSPKVEDFFGTHHNNTSHKEAMDLSLDSLFYNTTHEPNTTTNFQEFFSFPQTRNHEEETRNYGNDPSLTHGGSFNVGVYGEFQQSLSLSMSPGSQSSCITGSHHHQQNQNQNHQSQNHQQISEALVETSVGFETTTMAAAKKKRGQEDVVVVGQKQIVHRKSIDTFGQRTSQYRGVTRHRWTGRYEAHLWDNSFKKEGHSRKGRQVYLGGYDMEEKAARAYDLAALKYWGPSTHTNFSAENYQKEIEDMKNMTRQEYVAHLRRKSSGFSRGASIYRGVTRHHQHGRWQARIGRVAGNKDLYLGTFGTQEEAAEAYDVAAIKFRGTNAVTNFDITRYDVDRIMSSNTLLSGELARRNNNSIVVRNTEDQTALNAVVEGGSNKEVSTPERLLSFPAIFALPQVNQKMFGSNMGGNMSPWTSNPNAELKTVALTLPQMPVFAAWADS

SEQ ID NO: 93 - protein, 678 aa, Sorghum bicolor
MTNNNGNGTNAAASSWLGFSLSPHMASAMDEHHHVQQQQQHHHHHSLFFPSVTAAAAAAYGLGGSDGGVATSASPYYTPQLASMPLKSDGSLCIMEALRRSDQPDHHGPKLEDFLGAAAAQSQAMALSLQDNPAAAASSFYYYGNGGGGGSGHQHHGGFLQPCADLYGGPSEASLVADDDEAAAAATAMASWVAARAGESGGVLSAAAAAAGHQHHHHALALSMSSGSLSSCVTAHPGAAAADYGVVAATASASLDGGRKRGGAAGQKQPVHHRKSIDTFGQRTSQYRGVTRHRWTGRYEAHLWDNSCKKEGQTRKGRQGGYDMEEKAARAYDLAALKYWGPSTHINFPLEDYQEELEEMKNMTRQEYVAHLRRKSSGFSRGASMYRGVTRHHQHGRWQARIGRVSGNKDLYLGTFSTQEEAAEAYDIAAIKFRGLNAVTNFDITRYDVDKIMASNTLLPGDLARRRKDDDPAAVIAGADASNGGGVTTAAAAAALVQQAAAAAAAGAGGNHSASSSETWIKVAAAAALQAAGAAPRDGNHHHHHHDVLSGEAFSVLHDLVVTAADGGNGNGNGGHHHHHVHNSAATAQHMSMSSASSLVTSLGNSREGSPDRGGGLSMLFSKPPAPAPAASAHAANKPMSPLMPLGSWASTAAASARAAAAAVSIAHMPVFAAWTDA

SEQ ID NO: 94 - protein, 509 aa, Glycine max
MARASTNWLSFSLSPMDMLRTPEPQFVQYDAASDTSSHHYYLDNLYTNGWGNGSLKFEQNLNHSDVSFVQSSSQSVSHAPPKLEDFLGDSSAVMRYSDSQTETQDSSLTHIYDHHHHHHHGSSAYFGGDHQDLKAITGFQAFSTNSGSEVDDSASIGKAQGSEFGTHSIESSVNEFAAFSGGTNTGGTLSLAVAQSSEKAVAAAAESDRSKKVVDTFGQRTSIYRGVTRHRWTGRYEAHLWDNSCRREGQARKGRQGGYDKEEKAARSYDLAALKYWGPTATTNFPVSNYSKEVEEMKHVTKQEFIASLRRKSSGFSRGASIYRGVTRHHQQGRWQARIGRVAGNKDLYLGTFATEEEAAEAYDIAAIKFRGANAVTNFEMNRYDVEAIMKSSLPVGGAAKRLKLSLESEQKALPVSSSSSSSQQQNPQCGNVSASINFSSIHQPIASIPCGIPFDSTTAYYHHNLFQHFHPTNAGTAASAVTSANANALTALPPTAAAEFFIWPHQSY

SEQ ID NO: 95 - protein, 652 aa, Oryza sativa
MASGNSSSSSGSMAATAGGVGGWLGFSLSPHMATYCAGGVDDVGHHHHHHVHQHQQQHGGGLFYNPAAVASSFYYGGGHDAVVTSAAGGGSYYGAGFSSMPLKSDGSLCIMEALRGGDQEQQGVVVSASPKLEDFLGAGPAMALSLDNSAFYYGGHGHHQGHAQDGGAVGGDPHHGGGGFLQCAVIPGAGAGHDAALVHDQSAAAVAAGWAAMHGGGYDIANAAADDVCAAGPIIPTGGHLHPLTLSMSSAGSQSSCVTVQAAAAGEPYMAMDAVSKKRGGADRAGQKQPVHRKSIDTFGQRTSQYRGVTRHRWTGRYEAHLWDNSCKKEGQTRKGRQGGYDMEEKAARAYDLAALKYWGPSTHINFPLEDYQEELEEMKNMSRQEYVAHLRRKSSGFSRGASIYRGVTRHHQHGRWQARIGRVSGNKDLYLGTFSTQEEAAEAYDVAAIKFRGLNAVTNFDITRYDVDKILESSTLLPGELARRKGKVGDGGGAAAVADAAAALVQAGNVAEWKMATAAALPAAARTEQQQQHGHGGHQHHDLLPSDAFSVLQDIVSTVDAAGAPPRAPHMSMAATSLGNSREQSPDRGVGGGGGGGVLATLFAKPAAASKLYSPVPLNTWASPSPAVSSVPARAGVSIAHLPMFAAWTDA

SEQ ID NO: 96 - protein, 440 aa, Arabidopsis thaliana
MADSTTLSTFFDHSQTQIPKLEDFLGDSFVRYSDNQTETQDSSSLTPFYDPRHRTVAEGVTGFFSDHHQPDFKTINSGPEIFDDSTTSNIGGTHLSSHVVESSTTAKLGFNGDCTTTGGVLSLGVNNTSDQPLSCNNGERGGNSNKKKTVSKKETSDDSKKKIVETLGQRTSIYRGVTRHRWTGRYEAHLWDNSCRREGQARKGRQVYLGGYDKEDRAARAYDLAALKYWGSTATTNFPVSSYSKELEEMNHMTKQEFIASLRRKSSGFSRGASIYRGVTRHHQQGRWQARIGRVAGNKDLYLGTFATEEEAAEAYDIAAIKFRGINAVTNFEMNRYDIEAVMNSSLPVGGAAAKRHKLKLALESPSSSSSDHNLQQQQLLPSSSPSDQNPNSIPCGIPFEPSVLYYHQNFFQHYPLVSDSTIQAPMNQAEFFLWPNQSY

SEQ ID NO: 97 - protein, 651 aa, Zea mays
MANGSNWLGFSLSPHTAMEVPSVSEPASTHHAPPPPSSSTTISSSSTNNTISSNFLFSPMASPYPGYYCVGGAYGDGTSAAGVYYSHLPAMPNKSDDGTLCNMEGMVPSSPPKLEDFLGGGNGGGQETATYYSHQQQGQEEGASRDYRQYHYQHQQLVPYNFQPLTEAEMLQEGAAPMEEAMAAAKNFLLASYGACYSNEETRPLSLSMMSPGTQLSSCVSAAPQQQHQMAATVATAATAAAALGRSNGDGEQCVGRKRSTGKGGHKQTVHRKSIDTFGQRTSRYRGVTRHRWTGRYEAHLWDNSCRKDGQTRKGRQVYLGGYDTEDKAARAYDLAALKYWGPATHVNFPVENYRDELEEMKGMTRQEFVAHLRRRSSGFSRGASIYRGVTRHHQQGRWQSRIGRVAGNKDLYLGTFTTQEEAAEAYDIAAIKFRGLNAVTNFDIARYDVDKIMESSTLLAVEEARKVKAVEAASSAPMTHTHSGGKEQLNATTAEETSSAGWRMVLHGSPHQLEAARCPEAADLQSAIMNNDSHPRPSLHGIAGLDIECAVHDHHDHLDVPAGSRTTAAGSINFSNSSSQVTSLGNSREGSPERLGLAMMYGKQPSSAVSLAATMSPWTPVAAQTVAHVLKQQPNVVVSHRPVFAAWADA

SEQ ID NO: 98 - protein, 656 aa, Medicago truncatula
MKRMENNDDSVDINNENNWLGFSLSPQMNNIGVSSHTHHHSLPSATATASEVVPLQASFYHSSPLSNFCYSYGLEHENAGLYSLLPIMPLKSDGSLFEMEALSRSQTQAMSTTSAPKLENFLGNEAMGTPHYACSSTVTETMPLSLDSMFQNQIQQNMNMNNQQHLSYYNSTLRNHELMLEGSKQSQTSSGNFHQSNMGEDHGLSGLKNWVLRNFPASHGHDQSKMIVPVVEENEGECGSNIGSMAYGDLHSLSLSMSPSSQSSCVTTSQNMSSAVVENSVAMDTKKRGSEKFEQKQIVHRKSIDTFGQRTSQYRGVTRHRWTGRYEAHLWDNSCKKEGQSRKGRQGGYDMEEKAARAYDQAALKYWGPSTHINFPLENYQNQLEEMKNMTRQEYVAHLRRKSSGFSRGASMYRGVTSRHHQHGRWQARIGRVAGNKDLYLGTFSTQEEAAEAYDIAAIKFRGANAVTNFDIIKYDVEKIMASSNLLNIEQARRNKEVVDISSTQYIDQNKPSSAYDNNSTQEAISMQKSMVLYQSSQHQQLQQNQPRFENERTHQTFSSVSLDNMFHQEVVEEASKMRTHVSNASSLATSLSSSREGTPDRTSLQNLSGIMPSTASKLLVTSAPNSNLNSWDPSQHLRPSLSLPQMPVFAAWTDA

SEQ ID NO: 99 - protein, 546 aa, Glycine max
MKRMNESNNTDDGNNHNWLGFSLSPHMKMEVTSAATVSDNNVPTTFYMSPSHMSNSGMCYSVGENGNFHSPLTVMPLKSDGSLGILEALNRSQTQVMVPTSSPKLEDFLGGATMGTHEYGNHERGLSLDSIYYNSQNAEAQPNRNLLSHPFRQQGHAPSEEEATKETHVSVMPQMTGGGLQNWILEQQMNCGIWNERSGVSVGTVGCGELQSLSLSMSPGSQSSCVTAPSGTDSVAVDAKKRGHAKLGQKQPVHRKSIDTFGQRTSQYRGVTRHRWTGRYEAHLWDNSCKKEGQTRKGRQGGYDMEEKAARAYDLAALKYWGPSTHINFSIENYQVQLEEMKNMSRQEYVAHLRRKSSGFSRGASIYRGVTRHHQHGRWQARIGRVAGNKDLYLGTFSTQEEAAEAYDIAAIKFRGANAVTNFDISRYDVERIMASSNLLAGELARRNKDNDPRNEAIDYNKSVFKQETTMKMIRSGRCLSSSREASPEKMGPSLLFPKPPPMETKIVNPIGTSVTSWLPSPTVQMRPSPAISLSHLPVFAAWTDT

SEQ ID NO: 100 - protein, 415 aa, Arabidopsis thaliana
MKKWLGFSLTPPLRICNSEEEELRHDGSDVWRYDINFDHHHHDEDVPKVEDLLSNSHQTEYPINHNQTNVNCTTVVNRLNPPGYLLHDQTVVTPHYPNLDPNLSNDYGGFERVGSVSVFKSWLEQGTPAFPLSSHYVTEEAGTSNNISHFSNEETGYNTNGSMLSLALSHGACSDLINESNVSARVEEPVKVDEKRKRLVVKPQVKESVPRKSVDSYGQRTSQYRGVTRHRWTGRYEAHLWDNSCKKEGQTRRGRQVYLGGYDEEEKAARAYDLAALKYWGPTTHLNFPLSNYEKEIEELNNMNRQEFVAMLRRNSSGFSRGASVYRGVTRHHQHGRWQARIGRVAGNKDLYLGTFSTQEEAAEAYDIAAIKFRGLNAVTNFDINRYDVKRICSSSTIVDSDQAKHSPTSSGAGH

SEQ ID NO: 101 - protein, 428 aa, Zea mays
MSPPTNGAISLAYAPSMMLGAGALTNPPLLPFDGFTDEDFLASADAALLGEAGTDQTLLLLPSCPGANCCGGSSSDQGLGALACEVTTAGSFSLLGQPAPGQVSWEVTTAVAADRNTFSRARDPAPSPPPSPALPLVQTTSQSQRTSIYRGVTRHRWTGRYEAHLWDNTCRKEGQKRKGRQVYLGGYDKEDKAARAYDIAALKYWGDNATTNFPRENYIREIQDMQNMNRRDVVASLRRKSSGFSRGASIYRGVTKHHQHGRWQARIGRVAGNKDLYLGTFATEQEAAEAYDIAALKFRGENAVTNFEPSRYNLLAIAQRDIPILGRKLIQKPAPEAEDQAALSARSFSQSQQSSNSLPPYFLTNLLQPLPSQHSLAQALPSYNNLGFGEPSLYWPCPCGDPGEQKVQLGSKLEIVDGLVQLANSAAN

SEQ ID NO: 102 - protein, 438 aa, Arabidopsis thaliana
MKKRLTTSTCSSSPSSSVSSSTTTSSPIQSEAPRPKRAKRAKKSSPSGDKSHNPTSPASTRRSSIYRGVTRHRWTGRFEAHLWDKSSWNSIQNKKGKQVYLGAYDSEEAAAHTYDLAALKYWGPDTILNFPAETYTKELEEMQRVTKEEYLASLRRQSSGFSRGVSKYRGVARHHHNGRWEARIGRVFGNKYLYLGTYNTQEEAAAAYDMAAIEYRGANAVTNFDISNYIDRLKKKGVFPFPVNQANHQEGILVEAKQEVETREAKEEPREEVKQQYVEEPPQEEEEKEEEKAEQQEAEIVGYSEEAAVVNCCIDSSTIMEMDRCGDNNELAWNFCMMDTGFSPFLTDQNLANENPIEYPELFNELAFEDNIDFMFDDGKHECLNLENLDCCVVGRESPPSSSSPLSCLSTDSASSTTTTTTSVSCNYLFQGLFVGSE

SEQ ID NO: 103 - protein, 432 aa, Arabidopsis thaliana
MWDLNDAPHQTQREEESEEFCYSSPSKRVGSFSNSSSSAVVIEDGSDDDELNRVRPNNPLVTHQFFPEMDSNGGGVASGFPRAHWFGVKFCQSDLATGSSAGKATNVAAAVVEPAQPLKKSRRGPRSRSSQYRGVTFYRRTGRWESHIWDCGKQVYLGGFDTAHAAARAYDRAAIKFRGVEADINFNIDDYDDDLKQMTNLTKEEFVHVLRRQSTGFPRGSSKYRGVTLHKCGRWEARMGQFLGKKYVYLGLFDTEVEAARAYDKAAIKCNGKDAVTNFDPSIYDEELNAESSGNPTTPQDHNLDLSLGNSANSKHKSQDMRLRMNQQQQDSLHSNEVLGLGQTGMLNHTPNSNHQFPGSSNIGSGGGFSLFPAAENHRFDGRASTNQVLTNAAASSGFSPHHHNQIFNSTSTPHQNWLQTNGFQPPLMRPS

SEQ ID NO: 104 - protein, 449 aa, Arabidopsis thaliana
MLDLNLNADSPESTQYGGDSYLDRQTSDNSAGNRVEESGTSTSSVINADGDEDSCSTRAFTLSFDILKVGSSSGGDESPAASASVTKEFFPVSGDCGHLRDVEGSSSSRNWIDLSFDRIGDGETKLVTPVPTPAPVPAQVKKSRRGPRSRSSQYRGVTFYRRTGRWESHIWDCGKQVYLGGFDTAHAAARAYDRAAIKFRGVDADINFTLGDYEEDMKQVQNLSKEEFVHILRRQSTGFSRGSSKYRGVTLHKCGRWEARMGQFLGKKAYDKAAINTNGREAVTNFEMSSYQNEINSESNNSEIDLNLGISLSTGNAPKQNGRLFHFPSNTYETQRGVSLRIDNEYMGKPVNTPLPYGSSDHRLYWNGACPSYNNPAEGRATEKRSEAEGMMSNWGWQRPGQTSAVRPQPPGPQPPPLFSVAAASSGFSHFRPQPPNDNATRGYFYPHP

SEQ ID NO: 105 - DNA, 663 bp, Zea mays
  source 1..663: mol_type = genomic DNA
  CDS 1..663
atggaggcgctgagcgggcgggtaggcgtcaagtgcgggcggtggaaccctacggcggagcaggtgaaggtcctgacggagctcttccgcgcggggctgcggacgcccagcacggagcagatccagcgcatctccacccacctcagcgccttcggcaaggtggagagcaagaacgtcttctactggttccagaaccacaaggcccgcgagcgccaccaccacaagaagcgccgccgcggcgcgtcgtcgtcctcccccgacagcggcagcggcaggggaagcaacaacgaggaagacggccgtggtgccgcctcgcagtcgcacgacgccgacgccgacgccgacctcgtgctgcaaccgccagagagcaagcgggaggccagaagctatggccaccatcaccggctcgtgacatgctacgtcagggacgtggtggagcagcaggaggcgtcgccgtcgtgggagcggccgacgagggaggtggagacgctagagctcttccccctcaagtcgtacggcgacctcgaggcggcggagaaggtccggtcgtacgtcagaggcatcgccgccaccagcgagcagtgcagggagttgtccttcttcgacgtctccgccggccgggatccgccgctcgagctcaggctctgcagcttcggtccctag

SEQ ID NO: 106 - protein, 220 aa, Zea mays
MEALSGRVGVKCGRWNPTAEQVKVLTELFRAGLRTPSTEQIQRISTHLSAFGKVESKNVFYWFQNHKARERHHHKKRRRGASSSSPDSGSGRGSNNEEDGRGAASQSHDADADADLVLQPPESKREARSYGHHHRLVTCYVRDVVEQQEASPSWERPTREVETLELFPLKSYGDLEAAEKVRSYVRGIAATSEQCRELSFFDVSAGRDPPLELRLCSFGP

SEQ ID NO: 107 - DNA, 24 bp, synthetic construct
  misc_feature 1..24: note = attB1 variant
  source 1..24: mol_type = other DNA
caagttcgtacaaaaaagcaggct

SEQ ID NO: 108 - DNA, 23 bp, synthetic construct
  misc_feature 1..23: note = attB1 variant
  source 1..23: mol_type = other DNA
caagtttgtacaaaaaggactct

SEQ ID NO: 109 - DNA, 24 bp, synthetic construct
  misc_feature 1..24: note = attB1 variant
  source 1..24: mol_type = other DNA
caagtgcatacaaaaaggactgct

SEQ ID NO: 110 - DNA, 17747 bp, synthetic construct
  misc_feature 1..17747: note = PHP32371
  misc_feature 4310..4817: note = Rab17 promoter
  misc_feature 4818..4912: note = Rab17 5' UTR
  misc_feature 4951..4974: note = attB1
  misc_feature 5036..6496: note = FLP
  misc_feature 6513..6536: note = attB2
  misc_feature 6551..6868: note = PinII term
  source 1..17747: mol_type = other DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttaccagagctggtcacctttgtccaccaagatggaactgcggccgctcattaattaagtcaggcgcgcctctagttgaagacacgttcatgtcttcatcgtaagaagacactcagtagtcttcggccagaatggccatctggattcagcaggcctagaaggccatttaaatcctgaggatctggtcttcctaaggacccgggatatcgctatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccatggctagcgaagttcctattccgaagttcctattctctagaaagtataggaacttcagatctgccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgaagcggccgcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgatccgatatcgatgggccctggccgaagcttggtcacccggtccgggcctagaaggccagcttcaagtttgtacaaaaaagcaggctccggccagaatggcccggaccgggttaccgaattcgagctcggtaccctgggatccgatatcgatgggccctggccgaagctgggatatcgctatcaactttgtatagaaaagttgggccgaattcgagctcggtacggccagaatggcccggaccgggttaccgaattcgagctcggtaccctggggatccctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaagcttggtcacccggtccgggcctagaaggccagcttcaagtttgtacaaaaaagcaggcttcgaaggagatagaaccaattctctaaggaaatacttaaccatggtcgactggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagtttcgagatatctagacccagctttcttgtacaaagtggccgttaacggatccagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaagcttgcggccgccccagcttggtcacccggtccgggcctagaaggccgatctcccgggcacccagctttcttgtacaaagtggccgttaacggatcggccagaatggcccggaccgggttaccgaattcgagctcggtaccctgggatcgaccgaagctgaccgaagcttgcggccgcacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccgggggatctcgactctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattccggtcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaagcgtacgtgccggcctggctctccgaaagggcgaattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtcgatccgatatcgatgggccctggccgaagctaattcctgcagtgcagcgtgacccggtcgtgcccctctctagtggatctgagcttctagcgaagttcctattccgaagttcctattctctagaaagtataggaacttcagatctgcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctgaagcggccgcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttgcggccgccccgggcaactttattatacaaagttgatagatatcggaccgattaaactttaattcggtccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 111 - DNA, 17373 bp, synthetic construct
  misc_feature 1..17373: note = PHP35648
  misc_feature 4301..4808: note = Rab17 promoter
  misc_feature 4809..4903: note = Rab17 5' UTR
  misc_feature 4942..4965: note = attB1
  misc_feature 4991..6208: note = Cre
  misc_feature 6217..6240: note = attB2
  misc_feature 6255..6572: note = PinII term
  source 1..17373: mol_type = other DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttaccagagctggtcacctttgtccaccaagatggaactgcggccgctcattaattaagtcaggcgcgcctctagttgaagacacgttcatgtcttcatcgtaagaagacactcagtagtcttcggccagaatggccatctggattcagcaggcctagaaggccatttaaatcctgaggatctggtcttcctaaggacccgggatatcgctatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccatggctagcataacttcgtatagcatacattatacgaagttatccagatctgccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgaagcggccgcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgctgatccgatatcgatgggccctggccgaagcttggtcacccggtccgggcctagaaggccagcttcaagtttgtacaaaaaagcaggctccggccagaatggcccggaccgggttaccgaattcgagctcggtaccctgggatccgatatcgatgggccctggccgaagctgggatatcgctatcaactttgtatagaaaagttgggccgaattcgagctcggtacggccagaatggcccggaccgggttaccgaattcgagctcggtaccctggggatccctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaagcttggtcacccggtccgggcctagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatccagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaagcttgcggccgccccagcttggtcacccggtccgggcctagaaggccgatctcccgggcacccagctttcttgtacaaagtggccgttaacggatcggccagaatggcccggaccgggttaccgaattcgagctcggtaccctgggatcgaccgaagctgaccgaagcttgcggccgcacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccgggggatctcgactctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattccggtcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaagcgtacgtgccggcctggctctccgaaagggcgaattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtcgatccgatgatcctgagcttctagcataacttcgtatagcatacattatacgaagttatccagatctgcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctgaagcggccgcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttgcggccgccccgggcaactttattatacaaagttgatagatatcggaccgattaaactttaattcggtccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 112 - DNA, 11111 bp, synthetic construct
  misc_feature 1..11111: note = PHP46446
  misc_feature 1249..1756: note = Rab17 promoter
  misc_feature 1757..1851: note = Rab17 5' UTR
  misc_feature 1890..1913: note = attB1
  misc_feature 1939..3156: note = Cre
  misc_feature 3165..3188: note = attB2
  misc_feature 3203..3520: note = PinII term
  source 1..11111: mol_type = other DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcggctaatttaaatcctgaggatatcgctatcaactttgtatagaaaagttgggccgaattcgagctcggtacggccagaatggcccggaccgggttaccgaattcgagctcggtaccctgggatccgatatcgatgggccctggccgaagcttggtcacccggtccgggcctagaaggccagcttcaagtttgtacaaaaaagcaggctccggccagaatggcccggaccgggttaccgaattcgagctcggtaccctgggatccgatatcgatgggccctggccgaagcttggtcacccggtccgggcctagaaggccgatctcccgggcacccagctttcttgtacaaagtggccgttaacggatcggccagaatggcccggaccgggttaccgaattcgagctcggtaccctgggatccgatatcgatgggccctggccgaagcttggtcacccggtccgggcctagaaggccagcttcggccgccccgggcaactttattatacaaagttgatagataaatcctgaggatctggtcttcctaaggacccgggatatcggaccgattaaactttaattcggtccgacctggtggcgccgctagcgtatacgaagttcctattccgaagttcctattctccagaaagtataggaacttctgtacaataacttcgtatagcatacattatacgaagttatgcccgggctggtatttcaaaactatagtattttaaaattgcattaacaaacatgtcctaattggtactcctgagatactataccctcctgttttaaaatagttggcattatcgaattatcattttactctttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaagcttggtcacccggtccgggcctagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatccagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaagcttcggccgccccagcttggtcacccggtccgggcctagaaggccgatctcccgggcacccagctttcttgtacaaagtggccgttaacggatcggccagaatggcccggaccgggttaccgaattcgagctcggtaccctgggatcgaccgaagctgaccgaagcttgcggccgcacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccgggggatctcgactctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattcacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgcgactctagatcataatcagccataccacattcgaatgtgagttgatccccggcggtgtcccccactgaagaaactatgtgctgtagtatagccgctgcccgctggctagctagctagttgagtcatttagcggcgatgattgagtaataatgtgtcacgcatcaccatgcatgggtggcagtgtcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgagtcgacctcgagggggggcccggtaccaagatatcaaccgcggaaagatctaagcatgcaagggcccaagtcgacctgcagaagcttcggtccgggcctagaaggccgatctcccgggcacccagctttcttgtacaaagtggccgttaacggatcggccagaatggcccggaccgggttaccgaattcgagctcggtaccctgggatcgaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaagcgtacgtgccggcctggctctccgaaagggcgaattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacgtagaattgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 113 - DNA, 10477 bp, synthetic construct
  misc_feature 1..10477: note = PHP48733
  misc_feature 1306..1813: note = Rab17 promoter
  misc_feature 1814..1908: note = Rab17 5' UTR
  misc_feature 1947..1970: note = attB1
  misc_feature 1996..3213: note = Cre
  misc_feature 3222..3245: note = attB2
  misc_feature 3260..3577: note = PinII term
  source 1..10477: mol_type = other DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcggctaatttaaatcctgaggatatcgctatcaactttgtatagaaaagttgggccgaattcgagctcggtacggccagaatggcccggaccgggttaccgaattcgagctcggtaccctgggatccgatatcgatgggccctggccgaagcttggtcacccggtccgggcctagaaggccagcttcaagtttgtacaaaaaagcaggctccggccagaatggcccggaccgggttaccgaattcgagctcggtaccctgggatccgatatcgatgggccctggccgaagcttggtcacccggtccgggcctagaaggccgatctcccgggcacccagctttcttgtacaaagtggccgttaacggatcggccagaatggcccggaccgggttaccgaattcgagctcggtaccctgggatccgatatcgatgggccctggccgaagcttggtcacccggtccgggcctagaaggccagcttcggccgccccgggcaactttattatacaaagttgatagataaatcctgaggatctggtcttcctaaggacccgggatatcggaccgattaaactttaattcggtccgacctggtggcgccgctagcataacttcgtatagcatacattatacgaagttatccatatctgaccggcttaccgaatgcgtgctcgctaccctgggatgcgatttcgatggcccctggccgaagctggcatatcgctatgaccgggttagccaattcgacctcgctaccctggggatgctacgtaacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatgcagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaaggttgcgcccgccccagcttggtcagccgctccgggcttagaaggccgatctcgcgggcacccagctttcttctacaaagtggccgtttacggatcggctagaatgtcccgcaccggcttaccgatttgcacctcgctaccctgggttcgaccgaagctgaccgatgcttgcgcccgctcgcgaggccggccacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccggcggatctcgactctagaggatcgctcaggaaggccgctgagatagagccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacgtagaattgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 114 - DNA, 978 bp, Zea mays
  source 1..978: mol_type = genomic DNA
  CDS 1..978
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccttcaggcgcggcgcctccctcgcccaccctcggcttctacgccgccggcaatggcggcggatcggctgggctgctggacacgagttccgactggggcagcagcggcgctgctatggccaccgagacatgcttcctgcaggactacatgggcgtgacggacacgggcagctcgtcgcagtggccatgcttctcgtcgtcggacacgataatggcggcggcggcggccgcggcgcgggtggcgacgacgcgggcgcccgagacactccctctcttcccgacctgcggcgacgacgacgacgacgacagccagcccccgccgcggccgcggcacgcagtcccagtcccggcaggcgagaccatccgcggcggcggcggcagcagcagcagctacttgccgttctggggtgccggtgccgcgtccacaactgccggcgccacttcttccgttgcgatccagcagcaacaccagctgcaggagcagtacagcttttacagcaacagcacccagctggccggcaccggcagccaagacgtatcggcttcagcggccgccctggagctgagcctcagctcatggtgctccccttaccctgctgcagggagcatgtga

SEQ ID NO: 115 - protein, 325 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPSGAAPPSPTLGFYAAGNGGGSAGLLDTSSDWGSSGAAMATETCFLQDYMGVTDTGSSSQWPCFSSSDTIMAAAAAAARVATTRAPETLPLFPTCGDDDDDDSQPPPRPRHAVPVPAGETIRGGGGSSSSYLPFWGAGAASTTAGATSSVAIQQQHQLQEQYSFYSNSTQLAGTGSQDVSASAAALELSLSSWCSPYPAAGSM

SEQ ID NO: 116 - DNA, 3727 bp, Zea mays
  source 1..3727: mol_type = genomic DNA
atggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgatcaagaactggctgcggagccaaccggcgcccatgcagccgagggcggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggtgccgtcgtcgtcacggcgccgaaggaggatagcggtggcagcggtgttgccggtgctctagtagccgtgagcacggacacgggtggcagcggcggcgcgtcggctgacaacacggcaaggaagacggtggacacgttcgggcagcgcacgtcgatttaccgtggcgtgacaaggtaagggggtggatgaatcaagtaatcatgaaattttgaaaagccattggtaatccaaggaactgtcatgatagatttgattgcatctagacatagttccgatcgaatcaaatgagtaggccaatgtttagcctttggggatctcgctgattattaggagtaccattgtattgggcatggttgtggtatagtagtagacaattaacaaaaaagctaccacttttcaattattttaggcatagatggactgggagatatgaggcacatctttgggataacagttgcagaagggaaggacaaactcgtaagggtcgtcaaggtatacaaatataatgcaacatactgtcattaaatatgctttttctgtaagttttatatttcaccaatgatgttgttattgttaactgacattgcttcacactatcaattttggattcggcgcaatgatttgtgggattgaaatcaaatcttaaatctacagtctatttaggtacgcgatttctctccaactacttaatgcagttcgtttctccctataaccatattctttttcatctcaaatctcactcgactctttttttttatcttgtaccattgataggtggctatgataaagaggagaaagctgctagggcttatgatcttgctgctctgaagtactggggtcccacaacaacaacaaatttcccagtatgtatatgtagcatccagttttactttactgaagttcatatctcgttatgggctataaatatgtatcaaatgatgtccattagctagtgatctggagtgaaggttctatagtaaagtaaacgctgtgtgcggagtgcagtagcgggaggtctctcttctattttctaagaaaaatggacattgctgaaattgtacttaaagtcgtttattttatttttttgtatttccaggtgagtaactacgaaaaggagctcgaggacatgaagcacatgacaaggcaggagtttgtagcgtctctgagaaggtcggtctaacagcattgattaatcagtaccacctctactgaataaaatctgctgctatttgttaaattttgagcgaggtcaactgcatatttgatcttattagaccactgtatatgaatgcaggaagagcagtggtttctccagaggtgcatccatttacaggggagtgactaggtatgaattcatatagctaagaacttaacatcaacaaaaacacacatacacttgggttgatgtggcagatgcatgcatggattgaaaatgtgtgcatgttgttttacttgaactcgatctctgtatttataggcatcaccaacatggaagatggcaagcacggattggacgagttgcagggaacaaggatctttacttgggcaccttcagtaagtagcaaacaaatatgtttttgcattgtatatagagtacccttgaatatataaattcaccacatatacaagcaagttacagtcaactaacacaatctcaacgcaacgagaaagcaagtgttccagctgatagtacacatttgtagaccagccgcatatggttgttttgtatgcatgatgactattaaaaatgtgaccatcgcattaagtcatgcaaagttgcattgcagtagtacattgcttagtgcatgctcctcaagtggcttttttcaaacctgatcccatgtctggtgctattgttgtctcccattcacccgtgcatcaggtcaaaatagtaccatgcctgaataagaaaaacaaaacgagcatgcactggcagcagcagactaataaacaaagttccagcatttactaataaactaattaggctacagcatccaaaagattcttccaattaagccacaactgttcatgcatacatgggtatgccacccaggataccatgcatgcaccgtgcacgacgaaagcgaaacgctcgttctcggaatattagaactgacgaagccgagtgcaaccttctgtcgtggatgcaggcacccaggaggaggcagcggaggcgtacgacatcgcggcgatcaagttccgcggcctaaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagttacgacgtcggccgcatcgcctcgcagctcggcgacggcggagccctggcggcggcgtacggcgcgcactaccacggcgccgcctggccgaccatcgcgttccagccgggcgccgccaccacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaa

SEQ ID NO: 117 - DNA, 4325 bp, Oryza sativa
  source 1..4325: mol_type = genomic DNA
atgcatatctatcttatataaatatctaccagtgatactgttgcttagtgctccaaacctctcttgacctcttcttcttcttctcagttagcttagcttaagcttcccctaaccttgagctcaccacaacaatggcgacttgatctaacagagcttaaccaagtagcaaatcatacatataaccatagcttaattcgcattgaatcttgtcttgttcagtgtgaatcatcaaccatggccaccatgaacaactggctggccttctccctctccccgcaggatcagctcccgccgtctcagaccaactccactctcatctccgccgccgccaccaccaccaccgccggcgactcctccaccggcgacgtctgcttcaacatcccccaaggtaattaagctcaccaatcgatgcatgcattcatgagctagatatagctagtgttggttgggatttgaagagacatgcatgtttgattgattgatttgatgtgcagattggagcatgaggggatcggagctctcggcgctcgtcgccgagccgaagctggaggacttcctcggcggcatctccttctcggagcagcagcatcatcacggcggcaagggcggcgtgatcccgagcagcgccgccgcttgctacgcgagctccggcagcagcgtcggctacctgtaccctcctccaagctcatcctcgctccagttcgccgactccgtcatggtggccacctcctcgcccgtcgtcgcccacgacggcgtcagcggcggcggcatggtgagcgccgccgccgccgcggcggccagtggcaacggcggcattggcctgtccatgatcaagaactggctccggagccagccggcgccgcagccggcgcaggcgctgtctctgtccatgaacatggcggggacgacgacggcgcagggcggcggcgccatggcgctcctcgccggcgcaggggagcgaggccggacgacgcccgcgtcagagagcctgtccacgtcggcgcacggagcgacgacggcgacgatggctggtggtcgcaaggagattaacgaggaaggcagcggcagcgccggcgccgtggttgccgtcggctcggagtcaggcggcagcggcgccgtggtggaggccggcgcggcggcggcggcggcgaggaagtccgtcgacacgttcggccagagaacatcgatctaccgcggcgtgacaaggtatttagggtgcaattaattaatcatctatctatattttgctcaaaaaagttcatctactagctagcttagcacaaatcatcatcagtgtaatcatatatattctttgatgatttaactgtgttgcatgaattcattcctatttgatgtttgtgatttggatcccattttctaggatagctatataggtgatagattgatcattagatttgtaggatttatcattatgtcattattatgtgggacatgattgttgtgattaacaaagttgtaatatcttttggtttggttataggcatagatggacagggaggtatgaggctcatctttgggacaacagctgcagaagagagggccaaactcgcaagggtcgtcaaggtaggctaactagtgccatttaaatcgattaattgtttttttatgctccaatggcgattgatactgatcttgtttctttttctaatgatcatttcgggatcgaatgatcttcctctgtttgatcgaacttggcttttgaatctacagtctatctaggtgagtgagattccttgaacctagatgttctgtttgcgatgcatgtatatattcggtagattgaattatttgctgatctttgctttcttgaagtttaatgatcttataaattgtaatgctgataggtggttatgacaaagaggaaaaagctgctagagcttatgatttggctgctctcaaatactggggcccgacgacgacgacaaattttccggtgtgtttataattaatatacagattgtgtcacattgttattttctcactcttttatttgatactgatctagtgtaatgatgattactaaaactgtacttaaaggcaatggtttctgtatttttcaggtaaataactatgaaaaggagctggaggagatgaagcacatgacaaggcaggagttcgtagcctctttgagaaggttggtctctacaatcaagatatccatactatactaattaatttccttttagatttatagtaatttatctatcgcattgaagttaattaattatctgatgcttactgatactaacaaatactgttccttatatgtgcaggaagagcagtggtttctccagaggtgcatccatttaccgtggagtaactaggtacatatatatatgcatcattgtacaattaatttttttaatttttttagggtaaaaaatgaagactgtgatatagatccattaatttgatcttgtgtacttgtaaatataggcatcaccagcatgggagatggcaagcaaggataggaagagttgcagggaacaaggacctctacttgggcaccttcagtaagtacaaatattcatatttatactgcaaaaccatataaatccatattaataagtatgtcctttctcattgagtatacaaaatatcatattttcttggcaagtacaatttattcattcagggcaaaatagtagtagtaagaaagaggggtgactcttcaaagaacacagagcttacttaagcctgtaactaattaattaaactaaaaatgtgatctgcaagtcatgtcaagttgcattacaccactaatatatatactctgtgcatgcttgcatgctctcctcatgtggctagctaccttttcaaaccttccatgtctggtgctactcctgtctccattcaccactgcacctggtcaagatcctcactaattaagaaacaataatgcattatttgcagtaaataatttaactagtgttaatcacattctttgcaacacaaactaatcaccaattaagctagctagctagccaaaatgataatcttgcttgcatgcgctaatggtgtgtgtgatgatggtggtgtcacgcatgcaggcacgcaggaggaggcggcggaggcgtacgacatcgcggcgatcaagttccgggggctcaacgccgtcaccaacttcgacatgagccgctacgacgtcaagagcatcctcgacagcgctgccctccccgtcggcaccgccgccaagcgcctcaaggacgccgaggccgccgccgcctacgacgtcggccgcatcgcctcgcacctcggcggcgacggcgcctacgccgcgcattacggccaccaccaccactcggccgccgccgcctggccgaccatcgcgttccaggcggcggcggcgccgccgccgcacgccgccgggctttaccacccgtacgcgcagccgctgcgtgggtggtgcaagcaggagcaggaccacgccgtgatcgcggcggcgcacagcctgcaggatctccaccacctcaacctcggcgccgccgccgccgcgcatgacttcttctcgcaggcgatgcagcagcagcacggcctcggcagcatcgacaacgcgtcgctcgagcacagcaccggctccaactccgtcgtctacaacggcgacaatggcggcggaggcggcggctacatcatggcgccgatgagcgccgtgtcggccacggccaccgcggtggcgagcagccacgatcacggcggcgacggcgggaagcaggtgcagatggggtacgacagctacctcgtcggcgcagacgcctacggcggcggcggcgccgggaggatgccatcctgggcgatgacgccggcgtcggcgccggccgccacgagcagcagcgacatgaccggagtctgccatggcgcacagctcttcagcgtctggaacgacacataaaaaaaaaactaggttagccagcttaattagcagggtaaaccactgacacaattaagccatacttaaattagggttcatgagatgaccattaagcaggttattatcattaatgatgtttaatttctcaattagtacttagctcaaaaggaggggatttcttctgaaggatggtgatggcttgtgaaattgaacctggtgttcttgccatgatttttttttcacaagctgccattttggggttcaggttcagaaggatcctgattattattaaccagccatatatatatagaagggtagaaatggaggtatcctgcttgtaaattggggcaatggtagctagagttgatgcaatgaccatgcttcatgtgatgagaactaattgtcttcctctgatcaaattaagcaggaagattaa

SEQ ID NO: 118 - DNA, 2079 bp, Oryza sativa
  source 1..2079: mol_type = genomic DNA
  CDS 1..2079
atggccactatgaacaactggctcgccttctcgctctcgccgcaggaccaactcccaccgtcgcagaccaatagcactctcatctccgctgctgcaaccaccacaaccgcaggcgattcgtcaacgggcgacgtctgcttcaacatccctcaagactggtccatgcgcggaagcgagcttagcgctctcgtcgcggagcccaagttggaggatttcttgggaggcatctccttctcggagcaacagcatcatcacggcggaaagggcggtgttatcccaagctctgctgccgcatgctatgcaagctccggctccagcgtgggctacctctaccctccgccttcatcctcgtcacttcagtttgcagacagcgtgatggtcgcaacctcatctccagtggttgcgcacgatggcgtgagcggtggcggtatggtctcagcagcagcggctgcagcagcttcgggtaatggcgggattggcctctccatgatcaagaactggctcaggagccaaccggctccgcaacctgcgcaagcactcagcctgtcgatgaacatggctggtactactaccgctcaaggtggaggcgcaatggcacttctcgcaggcgctggcgaaagaggaaggaccacaccagcatccgagagcctctctacttccgcgcacggagccaccacggctacaatggctggcgggaggaaagagatcaacgaggaaggatctggatccgctggtgccgtggttgcagttggctcagaatcaggtggatccggcgctgttgttgaagctggtgccgctgcggcagcggctcggaagagcgttgatactttcggccaaagaacgagcatctacagaggcgttactcggcaccgctggaccggcaggtacgaggcacacttgtgggacaacagctgtcgccgcgagggccaaactaggaagggaagacagggaggatatgacaaagaggagaaggctgccagagcgtacgacctggccgcgttgaagtactggggtccaacaacgacgaccaacttcccggtgaacaactacgagaaggagctggaagagatgaagcacatgacgcggcaggagttcgtcgcttctctcaggcgcaagtcatctggtttctccagaggtgcgtcgatctatagaggagttacccgccaccaccagcacggaaggtggcaggcaagaatcgggagagtcgccggtaacaaggacctgtacttgggaaccttctcgactcaggaggaggcagcggaagcgtatgacattgcggcgatcaagttccgcggtctcaatgccgtgaccaacttcgacatgtcacgctatgatgtcaagtcgattctggatagcgctgcgttgcctgtgggaaccgctgccaaacgcctcaaggacgcggaagcagctgccgcgtacgatgttggcaggattgcctcacatctcggtggagatggagcttacgctgcccactacgggcatcatcaccactctgcagccgcagcttggcctacaatagcattccaagcggcagcggctcctcctccacacgctgctggtctttaccatccgtacgcgcaacctctccgcggttggtgtaagcaggaacaagatcatgcggtgattgcggctgcacacagcttgcaagatctgcatcacctcaatctgggagccgcagcagctgcccatgacttcttctcacaagccatgcagcagcagcatggcctgggcagcatagacaatgcgtctctggagcactccaccggatcgaactcggtggtgtacaatggagacaacggcggaggaggtggaggttacatcatggcacctatgtcagcggtctctgctaccgctacggcggtggcctcatcccacgaccacggtggagacggcggcaagcaggtccaaatgggctacgactcctaccttgtgggagctgacgcttacggcggaggaggagctggtcgcatgcctagctgggccatgacgcctgcttctgctcctgcggctacgagctcgtcggatatgacaggagtgtgtcatggcgcccaactgttctcggtgtggaatgatacatag

SEQ ID NO: 119 - protein, 692 aa, Oryza sativa
MATMNNWLAFSLSPQDQLPPSQTNSTLISAAATTTTAGDSSTGDVCFNIPQDWSMRGSELSALVAEPKLEDFLGGISFSEQQHHHGGKGGVIPSSAAACYASSGSSVGYLYPPPSSSSLQFADSVMVATSSPVVAHDGVSGGGMVSAAAAAAASGNGGIGLSMIKNWLRSQPAPQPAQALSLSMNMAGTTTAQGGGAMALLAGAGERGRTTPASESLSTSAHGATTATMAGGRKEINEEGSGSAGAVVAVGSESGGSGAVVEAGAAAAAARKSVDTFGQRTSIYRGVTRHRWTGRYEAHLWDNSCRREGQTRKGRQGGYDKEEKAARAYDLAALKYWGPTTTTNFPVNNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSAALPVGTAAKRLKDAEAAAAYDVGRIASHLGGDGAYAAHYGHHHHSAAAAWPTIAFQAAAAPPPHAAGLYHPYAQPLRGWCKQEQDHAVIAAAHSLQDLHHLNLGAAAAAHDFFSQAMQQQHGLGSIDNASLEHSTGSNSVVYNGDNGGGGGGYIMAPMSAVSATATAVASSHDHGGDGGKQVQMGYDSYLVGADAYGGGGAGRMPSWAMTPASAPAATSSSDMTGVCHGAQLFSVWNDT

SEQ ID NO: 120 - DNA, 2088 bp, Oryza sativa
  source 1..2088: mol_type = genomic DNA
  CDS 1..2088
atggccactatgaacaactggctcgccttctcgctctcgccgcaggaccaactcccaccgtcgcagaccaatagcactctcatctccgctgctgcaaccaccacaaccgcaggcgattcgtcaacgggcgacgtctgcttcaacatccctcaagactggtccatgcgcggaagcgagcttagcgctctcgtcgcggagcccaagttggaggatttcttgggaggcatctccttctcggagcaacagcatcatcacggcggaaagggcggtgttatcccaagctctgctgccgcatgctatgcaagctccggctccagcgtgggctacctctaccctccgccttcatcctcgtcacttcagtttgcagacagcgtgatggtcgcaacctcatctccagtggttgcgcacgatggcgtgagcggtggcggtatggtctcagcagcagcggctgcagcagcttcgggtaatggcgggattggcctctccatgatcaagaactggctcaggagccaaccggctccgcaacctgcgcaagcactcagcctgtcgatgaacatggctggtactactaccgctcaaggtggaggcgcaatggcacttctcgcaggcgctggcgaaagaggaaggaccacaccagcatccgagagcctctctacttccgcgcacggagccaccacggctacaatggctggcgggaggaaagagatcaacgaggaaggatctggatccgctggtgccgtggttgcagttggctcagaatcaggtggatccggcgctgttgttgaagctggtgccgctgcggcagcggctcggaagagcgttgatactttcggccaaagaacgagcatctacagaggcgttactcggcaccgctggaccggcaggtacgaggcacacttgtgggacaacagctgtcgccgcgagggccaaactaggaagggaagacaggtctatctaggaggatatgacaaagaggagaaggctgccagagcgtacgacctggccgcgttgaagtactggggtccaacaacgacgaccaacttcccggtgaacaactacgagaaggagctggaagagatgaagcacatgacgcggcaggagttcgtcgcttctctcaggcgcaagtcatctggtttctccagaggtgcgtcgatctatagaggagttacccgccaccaccagcacggaaggtggcaggcaagaatcgggagagtcgccggtaacaaggacctgtacttgggaaccttctcgactcaggaggaggcagcggaagcgtatgacattgcggcgatcaagttccgcggtctcaatgccgtgaccaacttcgacatgtcacgctatgatgtcaagtcgattctggatagcgctgcgttgcctgtgggaaccgctgccaaacgcctcaaggacgcggaagcagctgccgcgtacgatgttggcaggattgcctcacatctcggtggagatggagcttacgctgcccactacgggcatcatcaccactctgcagccgcagcttggcctacaatagcattccaagcggcagcggctcctcctccacacgctgctggtctttaccatccgtacgcgcaacctctccgcggttggtgtaagcaggaacaagatcatgcggtgattgcggctgcacacagcttgcaagatctgcatcacctcaatctgggagccgcagcagctgcccatgacttcttctcacaagccatgcagcagcagcatggcctgggcagcatagacaatgcgtctctggagcactccaccggatcgaactcggtggtgtacaatggagacaacggcggaggaggtggaggttacatcatggcacctatgtcagcggtctctgctaccgctacggcggtggcctcatcccacgaccacggtggagacggcggcaagcaggtccaaatgggctacgactcctaccttgtgggagctgacgcttacggcggaggaggagctggtcgcatgcctagctgggccatgacgcctgcttctgctcctgcggctacgagctcgtcggatatgacaggagtgtgtcatggcgcccaactgttctcggtgtggaatgatacatag

SEQ ID NO: 121 - DNA, 2133 bp, Zea mays
  source 1..2133: mol_type = genomic DNA
  CDS 1..2133
atggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaa

SEQ ID NO: 122 - protein, 710 aa, Zea mays
MATVNNWLAFSLSPQELPPSQTTDSTLISAATADHVSGDVCFNIPQDWSMRGSELSALVAEPKLEDFLGGISFSEQHHKANCNMIPSTSSTVCYASSGASTGYHHQLYHQPTSSALHFADSVMVASSAGVHDGGAMLSAAAANGVAGAASANGGGIGLSMIKNWLRSQPAPMQPRVAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAASTGLYHPYAQQPMRGGGWCKQEQDHAVIAAAHSLQDLHHLNLGAAGAHDFFSAGQQAAAAAMHGLGSIDSASLEHSTGSNSVVYNGGVGDSNGASAVGGSGGGYMMPMSAAGATTTSAMVSHEQVHARAYDEAKQAAQMGYESYLVNAENNGGGRMSAWGTVVSAAAAAAASSNDNMAADVGHGGAQLFSVWNDT

SEQ ID NO: 123 - DNA, 25 bp, synthetic construct
  misc_feature 1..25: note = Plasmid linker sequence
  source 1..25: mol_type = other DNA
tcgaaggagatagaaccgatccacc

SEQ ID NO: 124 - skipped

SEQ ID NO: 125 - DNA, 508 bp, Zea mays
  source 1..508: mol_type = genomic DNA
ctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctcgtgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgct

SEQ ID NO: 126 - DNA, 558 bp, Zea mays
  source 1..558: mol_type = genomic DNA
gtactgtaatatttatattatatataattataaactataatatttcaaaactatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctcgtgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgct